(19) United States
(12) Patent Application Publication
(10) Pub. No.: US 2025/0134974 A1
(43) Pub. Date: May 1, 2025

(54) MULTI-CHAIN CHIMERIC POLYPEPTIDES AND USE THEREOF IN THE TREATMENT OF ADVANCED SOLID TUMORS

(71) Applicant: ImmunityBio, Inc., San Diego, CA (US)

(72) Inventors: Hing C. Wong, San Diego, CA (US); Peter Rhode, San Diego, CA (US)

(21) Appl. No.: 18/932,350

(22) Filed: Oct. 30, 2024

Related U.S. Application Data
(60) Provisional application No. 63/594,483, filed on Oct. 31, 2023.

(51) Int. Cl. A61K 39/00; A61P 35/00; C07K 14/71

(52) U.S. Cl. CPC A61K 39/001103; A61P 35/00; C07K 14/71; C07K 2319/00

(57) ABSTRACT

Provided herein are multi-chain chimeric polypeptides and use thereof in the treatment of an unresectable, advanced, recurrent, and/or metastatic solid tumor or an unresectable, advanced, recurrent, and/or metastatic solid cancer.

### CROSS-REFERENCE TO RELATED APPLICATION

This application claims the benefit of priority under 35 U.S.C. § 119(e) to U.S. Provisional Application No. 63/594,483, filed Oct. 31, 2023. The entire disclosure is incorporated herein by reference.

### REFERENCE TO A SEQUENCE LISTING

This application contains a Sequence Listing submitted electronically as an XML file and is hereby incorporated by reference in its entirety. Said XML file, created on Oct. 29, 2024, is named “8774HCW-16.xml” and is 53,689 bytes in size.

### TECHNICAL FIELD

The present disclosure relates to the field of biotechnology, and more specifically, to multi-chain chimeric polypeptides and methods of treating advanced solid tumors and advanced solid cancer.

### BACKGROUND

Tissue factor (TF), a 263 amino acid integral membrane glycoprotein with a molecular weight of ˜46 kDa and the trigger protein of the extrinsic blood coagulation pathway, is the primary initiator of coagulation in vivo. Tissue factor, normally not in contact with circulating blood, initiates the coagulation cascade upon exposure to the circulating coagulation serine protease factors. Vascular damage exposes sub-endothelial cells expressing tissue factor, resulting in the formation of a calcium-dependent, high-affinity complex with pre-existing plasma factor VIIa (FVIIa). Binding of the serine protease FVIIa to tissue factor promotes rapid cleavage of FX to FXa and FIX to FIXa. The proteolytic activity of the resulting FXa and an active membrane surface then inefficiently converts a small amount of prothrombin to thrombin. The thrombin generated by FXa initiates platelet activation and activates minute amounts of the pro-cofactors factor V (FV) and factor VIII (FVIII) to become active cofactors, factor Va (FVa) and factor VIIIa (FVIIIa). FIXa complexes with FVIIIa on the platelet surface forming the intrinsic tenase complex, which results in rapid generation of FXa. FXa complexes with FVa to form the pro-thrombinase complex on the activated platelet surface which results in rapid cleavage of prothrombin to thrombin.

In addition to the tissue factor-FVIIa complex, a recent study showed that the tissue factor-FVIIa-FXa complex can activate FVIII, which would provide additional levels of FVIIIa during the initiation phase. The extrinsic pathway is paramount in initiating coagulation via the activation of limited amounts of thrombin, whereas the intrinsic pathway maintains coagulation by dramatic amplification of the initial signal.

Much of the tissue factor expressed on a cell surface is “encrypted,” which must be “decrypted” for full participation in coagulation. The mechanism of “decryption” of cell-surface tissue factor is still unclear at this time, however, exposure of anionic phospholipids plays a major role in this process. Healthy cells actively sequester anionic phospholipids such as phosphatidyl serine (PS) to the inner leaflet of the plasma membrane. Following cellular damage, activation, or increased levels of cytosolic Ca²⁺, this bilayer asymmetry is lost, resulting in increased PS exposure on the outer leaflet, which increases the specific activity of cell-surface tissue factor-FVIIa complexes. PS exposure is known to decrease the apparent Km for activation of FIX and FX by tissue factor-FVIIa complexes, but additional mechanisms could include conformational rearrangement of tissue factor or tissue factor-FVIIa and subsequent exposure of substrate binding sites.

### SUMMARY

Provided herein are methods and/or uses of the compositions disclosed herein for treating an unresectable, advanced, recurrent, and/or metastatic solid cancer or an unresectable, advanced, recurrent, and/or metastatic solid tumor in a subject that include administering to the subject two or more doses of a multi-chain chimeric polypeptide, wherein the time between administration of any two consecutive doses of the two or more doses is 15 days to 27 days, wherein the multi-chain chimeric polypeptide comprises: (a) a first chimeric polypeptide comprising: (i) a first target-binding domain; (ii) a soluble tissue factor domain comprising a sequence that is at least 90% identical to SEQ ID NO:1; and (iii) a first domain of a pair of affinity domains comprising a sequence that is at least 90% to SEQ ID NO:13; and (b) a second chimeric polypeptide comprising: (i) a second domain of a pair of affinity domains that is at least 90% identical to SEQ ID NO:11; and (ii) a second target-binding domain, wherein: the first chimeric polypeptide and the second chimeric polypeptide associate through the binding of the first domain and the second domain of the pair of affinity domains; and the first target-binding domain bind and the second target-binding domain each comprises a soluble TGF-β receptor II (TGF-βRII) and each comprises a sequence that is at least 90% identical to SEQ ID NO:4.

Also provided herein are compositions that include any of the multi-chain chimeric polypeptides described herein. In some embodiments of any of the compositions described herein, the composition is a pharmaceutical composition.

In some embodiments of any of the methods and/or uses of the compositions described herein, the subject has previously received a previous treatment with a standard first or later line of systemic therapy for a solid tumor, and the subject's solid tumor had recurred or progressed and/or the subject was intolerant to the previous treatment.

In some embodiments of any of the methods described herein, the subject has been previous identified or diagnosed as having an unresectable, advanced, recurrent, and/or metastatic solid tumor or unresectable, advanced, recurrent, and/or metastatic solid cancer.

In some embodiments of any of the methods and/or uses of the compositions described herein, the unresectable, advanced, recurrent, and/or metastatic solid tumor or the unresectable, advanced, recurrent, and/or metastatic solid cancer is selected from the group consisting of: bladder cancer, gastric cancer, breast cancer, cervical cancer, colorectal cancer, endometrial cancer, kidney cancer, lip cancer, liver cancer, melanoma, rhabdomyosarcoma, Ewing sarcoma, mesothelioma, lung cancer, non-small cell lung cancer, non-melanoma skin cancer, oral cancer, ovarian cancer, pancreatic cancer, sarcoma, osteosarcoma, glioblastoma, neuroblastoma, small cell lung cancer, thyroid cancer, retinoblastoma, urothelial carcinoma, renal cell carcinoma, esophageal cancer, prostate cancer, squamous cell head and neck carcinoma, hepatocellular cancer, testicular cancer, penile cancer, uterine cancer, vaginal cancer, gallbladder cancer, Merkel cell carcinoma, gastroesophageal junction cancer, microsatellite instability-high (MSI-H) solid tumor, a mismatch repair deficient (dMMR) solid tumor, and tumor mutational burden-high (TMB-H) solid tumor.

In some embodiments of any of the methods and/or uses of the compositions described herein, the cancer is ovarian cancer, colorectal cancer or liver cancer.

In some embodiments of any of the methods and/or uses of the compositions described herein, the time between administration of any two consecutive doses of the two or more doses is 18 days to 24 days. In some embodiments of any of the methods described herein, the time between administration of any two consecutive doses of the two or more doses is about 19 days to about 23 days. In some embodiments of any of the methods described herein, the time between administration of any two consecutive doses of the two or more doses is about 20 days to about 22 days. In some embodiments of any of the methods described herein, the time between administration of any two consecutive doses of the two or more doses is 21 days.

In some embodiments of any of the methods and/or uses of the compositions described herein, each of the two or more doses is 0.2 mg/kg to 1.3 mg/kg. In some embodiments of any of the methods described herein, each of the two or more doses is 0.25 mg/kg to 1.2 mg/kg/ In some embodiments of any of the methods described herein, each of the two or more doses is 0.4 mg/kg to 0.6 mg/kg. In some embodiments of any of the methods described herein, each of the two or more doses is 0.5 mg/kg

In some embodiments of any of the methods and/or uses of the compositions described herein, each of the two or more doses is 0.7 mg/kg to 0.9 mg/kg. In some embodiments of any of the methods described herein, each of the two or more doses is 0.8 mg/mg.

In some embodiments of any of the methods and/or uses of the compositions described herein, each of the two or more doses is 1.1 mg/kg to 1.3 mg/kg. In some embodiments of any of the methods described herein, each of the two or more doses is 1.2 mg/mg.

In some embodiments of any of the methods and/or uses of the compositions described herein, each of the two or more doses is 20 mg to 150 mg.

In some embodiments of any of the methods and/or uses of the compositions described herein, each of the two or more doses is 25 mg to 150 mg. In some embodiments of any of the methods described herein, each of the two or more doses is 35 mg to 150 mg. In some embodiments of any of the methods and/or uses of the compositions described herein, each of the two or more doses is 40 mg to 150 mg. In some embodiments of any of the methods and/or uses of the compositions described herein, each of the two or more doses is 50 mg to 150 mg. In some embodiments of any of the methods described herein, each of the two or more doses is 60 mg to 150 mg. In some embodiments of any of the methods and/or uses of the compositions described herein, each of the two or more doses is 60 mg to 100 mg. In some embodiments of any of the methods and/or uses of the compositions described herein, each of the two or more doses is 80 mg to 150 mg. In some embodiments of any of the methods and/or uses of the compositions described herein, each of the two or more doses is 100 mg to 150 mg.

In some embodiments of any of the methods and/or uses of the compositions described herein, the two or more doses is at least three doses. In some embodiments of any of the methods and/or uses of the compositions described herein, the two or more doses is at least four doses. In some embodiments of any of the methods and/or uses of the compositions described herein, the two or more doses is at least six doses. In some embodiments of any of the methods and/or uses of the compositions described herein, the two or more doses is at least eight doses. In some embodiments of any of the methods and/or uses of the compositions described herein, the two or more doses is at least ten doses.

In some embodiments of any of the methods and/or uses of the compositions described herein, the two or more doses are formulated for subcutaneous administration. In some embodiments of any of the methods and/or uses of the compositions described herein, the two or more doses are administered to the subject using subcutaneous administration.

In some embodiments of any of the methods and/or uses of the compositions described herein, the two or more doses are formulated for intravenous administration. In some embodiments of any of the methods and/or uses of the compositions described herein, the two or more doses are administered to the subject using intravenous administration.

In some embodiments of any of the methods and/or uses of the compositions described herein, the first target-binding domain and the soluble tissue factor domain directly abut each other in the first chimeric polypeptide. In some embodiments of any of the methods and/or uses of the compositions described herein, the first chimeric polypeptide further comprises a linker sequence between the first target-binding domain and the soluble tissue factor domain in the first chimeric polypeptide. In some embodiments of any of the methods and/or uses of the compositions described herein, the soluble tissue factor domain and the first domain of the pair of affinity domains directly abut each other in the first chimeric polypeptide. In some embodiments of any of the methods and/or uses of the compositions described herein, the first chimeric polypeptide further comprises a linker sequence between the soluble tissue factor domain and the first domain of the pair of affinity domains in the first chimeric polypeptide.

In some embodiments of any of the methods and/or uses of the compositions described herein, the second domain of the pair of affinity domains and the second target-binding domain directly abut each other in the second chimeric polypeptide. In some embodiments of any of the methods and/or uses of the compositions described herein, the second chimeric polypeptide further comprises a linker sequence between the second domain of the pair of affinity domains and the second target-binding domain in the second chimeric polypeptide.

In some embodiments of any of the methods and/or uses of the compositions described herein, one or both of the first target-binding domain and the second target-binding domain is an antigen-binding domain. In some embodiments of any of the methods and/or uses of the compositions described herein, one or both of the first target-binding domain and the second target-binding domain is a soluble interleukin or cytokine receptor.

In some embodiments of any of the methods and/or uses of the compositions described herein, the first chimeric polypeptide further comprises one or more additional target-binding domain(s). In some embodiments of any of the methods and/or uses of the compositions described herein, the second chimeric polypeptide further comprises one or more additional target-binding domain(s).

In some embodiments of any of the methods and/or uses of the compositions described herein, the soluble tissue factor domain is a soluble human tissue factor domain. In some embodiments of any of the methods and/or uses of the compositions described herein, the soluble human tissue factor domain comprises a sequence that is at least 90% identical to SEQ ID NO: 1. In some embodiments of any of the methods and/or uses of the compositions described herein, the pair of affinity domains is a sushi domain from an alpha chain of human IL-15 receptor (IL-15Rα) and a soluble IL-15.

In some embodiments of any of the methods and/or uses of the compositions described herein, the first target-binding domain comprises a soluble TGF-βRII. In some embodiments of any of the methods described herein, the first target-binding domain comprises a sequence of SEQ ID NO:4.

In some embodiments of any of the methods and/or uses of the compositions described herein, the first chimeric polypeptide comprises a sequence that is at least 80% identical to SEQ ID NO:6. In some embodiments of any of the methods and/or uses of the compositions described herein, the first chimeric polypeptide comprises a sequence that is at least 90% identical to SEQ ID NO:6. In some embodiments of any of the methods and/or uses of the compositions described herein, the first chimeric polypeptide comprises a sequence of SEQ ID NO:6.

In some embodiments of any of the methods and/or uses of the compositions described herein, the second target-binding domain comprises a soluble TGF-βRII. In some embodiments of any of the methods described herein, the second target-binding domain comprises a sequence of SEQ ID NO:4.

In some embodiments of any of the methods and/or uses of the compositions described herein, the second chimeric polypeptide comprises a sequence that is at least 80% identical to SEQ ID NO:5. In some embodiments of any of the methods and/or uses of the compositions described herein, the first chimeric polypeptide comprises a sequence that is at least 80% identical to SEQ ID NO:6. In some embodiments of any of the methods and/or uses of the compositions described herein, the second chimeric polypeptide comprises a sequence that is at least 90% identical to SEQ ID NO:5. In some embodiments of any of the methods and/or uses of the compositions described herein, the second chimeric polypeptide comprises a sequence of SEQ ID NO:5. In some embodiments of any of the methods and/or uses of the compositions described herein, the first chimeric polypeptide comprises a sequence of SEQ ID NO:6.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### BRIEF DESCRIPTION OF DRAWINGS

FIG. 1 shows exemplary diagrams for a multi-chain chimeric polypeptide: (i) a first chimeric polypeptide including a first target-binding domain (A), a soluble tissue factor domain, a first domain of an affinity pair of domains (soluble interleukin IL-15), and an additional target-binding domain (B); and (ii) second chimeric polypeptide including a second domain of an affinity pair of domains (IL-15 receptor alpha sushi domain), a second target-binding domain (C), and an additional antigen-binding domain (D). The top cartoon diagram depicts the association of the first and the second chimeric polypeptides through the pair of affinity domains. The bottom schematic diagrams show the order of the domains in the first and second chimeric polypeptides.

FIG. 2 shows exemplary diagrams for a multi-chain chimeric polypeptide: (i) a first chimeric polypeptide including a first target-binding domain (A), a soluble tissue factor domain including five amino acid substitutions in order to remove binding of the soluble tissue factor domain to FVIIa, a first domain of an affinity pair of domains (soluble interleukin IL-15 including a D8N or D8A amino acid substitution), and an additional target-binding domain (B); and (ii) second chimeric polypeptide including a second domain of an affinity pair of domains (IL-15 receptor alpha sushi domain), a second target-binding domain (C), and an additional antigen-binding domain (D). The top cartoon diagram depicts the association of the first and the second chimeric polypeptides through the pair of affinity domains. The bottom schematic diagrams show the order of the domains in the first and second chimeric polypeptides. In other embodiments of any of the multi-chain chimeric polypeptides described herein the soluble tissue factor domain can comprise or consists of a soluble wildtype human tissue factor domain (comprising or consisting of a contiguous sequence within wildtype human tissue factor).

FIG. 3 shows a schematic of the TGFRt15-TGFRs construct.

FIG. 4 shows an additional schematic of the TGFRt15-TGFRs construct.

FIG. 5 shows the effect of TGFRt15-TGFRs (HCW9218) from chemical-induced liver damage.

FIG. 6 shows an exemplary schematic of the administration cycle and dose levels of the clinical trial of TGFRt15-TGFRs (HCW9218) in patients with chemo-refractory/resistant advanced solid tumors.

FIG. 7 shows neutralization of TGF-β1 and TGF-β2 by TGFRt15-TGFRs (HCW9218) dose level in subjects with solid tumors. TGFRt15-TGFRs (HCW9218) dose-dependent reduction in serum TGF-β1 and TGF-β2 levels (to baseline at >0.5 mg/kg HCW9218) were observed.

FIG. 8A shows percentages of blood Ki67⁺ NK cells in subjects with solid tumors following treatment with TGFRt15-TGFRs (HCW9218) as assessed by flow cytometry. FIG. 8B shows percentages of blood CD8⁺ T cells TGFRt15-TGFRs (HCW9218) by flow cytometry. All subjects had a robust proliferation of blood NK cells, ranging from 77% to 97% Ki67-positivity by Day 8 after dosing for each treatment cycle. TGFRt15-TGFRs (HCW9218)-mediated increases in blood NK cell percentages and counts were also observed. Treatment induction for blood CD8⁺ T cell proliferation was also observed. Responses were sustained through Day 15, a biological effect beyond that previously observed for other IL-15 agonists.

FIG. 9A shows absolute number of blood NK cells in subjects with solid tumors following treatment with TGFRt15-TGFRs (HCW9218). FIG. 9B shows absolute number of blood CD8⁺ cells in subjects with solid tumors following treatment with TGFRt15-TGFRs (HCW9218). FIG. 9C shows absolute number of blood CD4⁺ T cells in subjects with solid tumors following treatment with TGFRt15-TGFRs (HCW9218).

FIG. 10 shows immune cell staining in pre- and post-treatment tumor biopsy specimens from subject with ovarian cancer following treatment with TGFRt15-TGFRs (HCW9218).

FIGS. 11A-11B show percentage of blood CD8⁺ T cells that are PD-1⁺ and TCF1⁺ Eomes⁺ in subjects with solid tumors following treatment with TGFRt15-TGFRs (HCW9218). FIG. 11A shows presence of exhausted (PD1⁺) CD8⁺ T cells and FIG. 11B shows Tpex (TCF1⁺) CD8⁺ T cells, when evaluated using time of flight mass cytometry (CyTOF) using Maxpar Direct Immune Profiling assay with a NK cell expansion panel (for dose escalation) or a custom CD8⁺ T cell expansion panel (for the extension cohort) and then analyzed with Maxpar Pathsetter software.

### DETAILED DESCRIPTION

Provided herein are methods of treating an unresectable, advanced, recurrent, and/or metastatic solid cancer or an unresectable, advanced, recurrent, and/or metastatic solid tumor (e.g., any of the exemplary unresectable, advanced, recurrent, and/or metastatic solid tumors or unresectable, advanced, recurrent, and/or metastatic solid cancers described herein or known in the art) in a subject, the method comprising administering to the subject two or more doses of about 0.2 mg/kg to about 1.3 mg/kg (e.g., any of the subranges of this range described herein or any of the values in this range described herein) of a multi-chain chimeric polypeptide, wherein the time between administration of any two consecutive doses of the two or more doses is about 15 days to about 27 days (e.g., any of the subranges of this range described herein or any of the values in this range described herein), wherein the multi-chain chimeric polypeptide comprises: (a) a first chimeric polypeptide comprising: (i) a first target-binding domain; (ii) a soluble tissue factor domain; and (iii) a first domain of a pair of affinity domains; (b) a second chimeric polypeptide comprising: (i) a second domain of a pair of affinity domains; and (ii) a second target-binding domain, wherein: the first chimeric polypeptide and the second chimeric polypeptide associate through the binding of the first domain and the second domain of the pair of affinity domains; and the first target-binding domain bind and the second target-binding domain each comprises a soluble TGF-β receptor II (TGF-βRII) and each comprises a sequence that is at least 90% identical to SEQ ID NO:4.

Also provided herein are methods of treating an unresectable, advanced, recurrent, and/or metastatic solid cancer or an unresectable, advanced, recurrent, and/or metastatic solid tumor (e.g., any of the exemplary unresectable, advanced, recurrent, and/or metastatic solid tumors or unresectable, advanced, recurrent, and/or metastatic solid cancers described herein or known in the art) in a subject, the method comprising administering to the subject two or more doses of about 20 mg to about 150 mg (e.g., any of the subranges of this range described herein or any of the values in this range described herein) of a multi-chain chimeric polypeptide, wherein the time between administration of any two consecutive doses of the two or more doses is about 15 days to about 27 days (e.g., any of the subranges of this range described herein or any of the values in this range described herein), wherein the multi-chain chimeric polypeptide comprises: (a) a first chimeric polypeptide comprising: (i) a first target-binding domain; (ii) a soluble tissue factor domain; and (iii) a first domain of a pair of affinity domains; (b) a second chimeric polypeptide comprising: (i) a second domain of a pair of affinity domains; and (ii) a second target-binding domain, wherein: the first chimeric polypeptide and the second chimeric polypeptide associate through the binding of the first domain and the second domain of the pair of affinity domains; and the first target-binding domain bind and the second target-binding domain each comprises a soluble TGF-β receptor II (TGF-βRII) and each comprises a sequence that is at least 90% identical to SEQ ID NO:4.

As used herein, the term “chimeric” refers to a polypeptide that includes amino acid sequences (e.g., domains) originally derived from two different sources (e.g., two different naturally-occurring proteins, e.g., from the same or different species). For example, a chimeric polypeptide can include domains from at least two different naturally occurring human proteins. In some examples, a chimeric polypeptide can include a domain that is a synthetic sequence (e.g., an scFv) and a domain that is derived from a naturally-occurring protein (e.g., a naturally-occurring human protein). In some embodiments, a chimeric polypeptide can include at least two different domains that are synthetic sequences (e.g., two different scFvs).

An “antigen-binding domain” is one or more protein domain(s) (e.g., formed from amino acids from a single polypeptide or formed from amino acids from two or more polypeptides (e.g., the same or different polypeptides) that is capable of specifically binding to one or more different antigen(s). In some examples, an antigen-binding domain can bind to an antigen or epitope with specificity and affinity similar to that of naturally-occurring antibodies. In some embodiments, the antigen-binding domain can be an antibody or a fragment thereof. In some embodiments, an antigen-binding domain can include an alternative scaffold. Non-limiting examples of antigen-binding domains are described herein. Additional examples of antigen-binding domains are known in the art.

A “soluble tissue factor domain” refers to a polypeptide having at least 70% identity (e.g., at least 75% identity, at least 80% identity, at least 85% identity, at least 90% identity, at least 95% identity, at least 99% identity, or 100% identical) to a segment of a wildtype mammalian tissue factor protein (e.g., a wildtype human tissue factor protein) that lacks the transmembrane domain and the intracellular domain. Non-limiting examples of soluble tissue factor domains are described herein.

The term “soluble interleukin receptor” is used herein in the broadest sense to refer to a polypeptide that lacks a transmembrane domain (and optionally an intracellular domain) that is capable of binding one or more of its natural ligands (e.g., under physiological conditions, e.g., in phosphate buffered saline at room temperature). For example, a soluble interleukin receptor can include a sequence that is at least 70% identical (e.g., at least 75% identical, at least 80% identical, at least 85% identical, at least 90% identical, at least 95% identical, at least 99% identical, or 100% identical) to an extracellular domain of wildtype interleukin receptor and retains its ability to specifically bind to one or more of its natural ligands, but lacks its transmembrane domain (and optionally, further lacks its intracellular domain). Non-limiting examples of soluble interleukin receptors are described herein.

The term “soluble cytokine receptor” is used herein in the broadest sense to refer to a polypeptide that lacks a transmembrane domain (and optionally an intracellular domain) that is capable of binding one or more of its natural ligands (e.g., under physiological conditions, e.g., in phosphate buffered saline at room temperature). For example, a soluble cytokine receptor can include a sequence that is at least 70% identical (e.g., at least 75% identical, at least 80% identical, at least 85% identical, at least 90% identical, at least 95% identical, at least 99% identical, or 100% identical) to an extracellular domain of wildtype cytokine receptor and retains its ability to specifically bind to one or more of its natural ligands, but lacks its transmembrane domain (and optionally, further lacks its intracellular domain). Non-limiting examples of soluble cytokine receptors are described herein.

The term “antibody” is used herein in its broadest sense and includes certain types of immunoglobulin molecules that include one or more antigen-binding domains that specifically bind to an antigen or epitope. An antibody specifically includes, e.g., intact antibodies (e.g., intact immunoglobulins), antibody fragments, and multi-specific antibodies. One example of an antigen-binding domain is an antigen-binding domain formed by a VH-VL dimer. Additional examples of an antibody are described herein. Additional examples of an antibody are known in the art.

“Affinity” refers to the strength of the sum total of non-covalent interactions between an antigen-binding site and its binding partner (e.g., an antigen or epitope). Unless indicated otherwise, as used herein, “affinity” refers to intrinsic binding affinity, which reflects a 1:1 interaction between members of an antigen-binding domain and an antigen or epitope. The affinity of a molecule X for its partner Y can be represented by the dissociation equilibrium constant (K_{D}). The kinetic components that contribute to the dissociation equilibrium constant are described in more detail below. Affinity can be measured by common methods known in the art, including those described herein. Affinity can be determined, for example, using surface plasmon resonance (SPR) technology (e.g., BIACORE®) or biolayer interferometry (e.g., FORTEBIO®). Additional methods for determining the affinity for an antigen-binding domain and its corresponding antigen or epitope are known in the art.

A “multi-chain polypeptide” as used herein to refers to a polypeptide comprising two or more (e.g., three, four, five, six, seven, eight, nine, or ten) protein chains (e.g., at least a first chimeric polypeptide and a second polypeptide), where the two or more proteins chains associate through non-covalent bonds to form a quaternary structure.

The term “pair of affinity domains” is two different protein domain(s) that bind specifically to each other with a K_{D} of less than of less than 1×10⁻⁷ M (e.g., less than 1×10⁻⁸ M, less than 1×10⁻⁹ M, less than 1×10⁻¹⁰ M, or less than 1×10⁻¹¹ M). In some examples, a pair of affinity domains can be a pair of naturally-occurring proteins. In some embodiments, a pair of affinity domains can be a pair of synthetic proteins. Non-limiting examples of pairs of affinity domains are described herein.

The term “epitope” means a portion of an antigen that specifically binds to an antigen-binding domain. Epitopes can, e.g., consist of surface-accessible amino acid residues and/or sugar side chains and may have specific three-dimensional structural characteristics, as well as specific charge characteristics. Conformational and non-conformational epitopes are distinguished in that the binding to the former but not the latter may be lost in the presence of denaturing solvents. An epitope may comprise amino acid residues that are directly involved in the binding, and other amino acid residues, which are not directly involved in the binding. Methods for identifying an epitope to which an antigen-binding domain binds are known in the art.

An “immune effector cell” refers to a cell of the immune system of a mammal that is capable, directly or indirectly, of recognizing and/or causing cytostasis or cell death of a pathogenic cell (e.g., a cancer cell) in the mammal. Non-limiting examples of immune effector cells include macrophages, T-lymphocytes (e.g., cytotoxic T-lymphocytes and T-helper cells), natural killer cells, neutrophils, monocytes, and eosinophils. Additional examples of immune effector cells are known in the art.

The term “treatment” means to ameliorate at least one symptom of a disorder. In some examples, the disorder being treated is cancer and to ameliorate at least one symptom of cancer includes reducing aberrant proliferation, gene expression, signaling, translation, and/or secretion of factors. Generally, the methods of treatment include administering a therapeutically effective amount of composition that reduces at least one symptom of a disorder to a subject who is in need of, or who has been determined to be in need of such treatment.

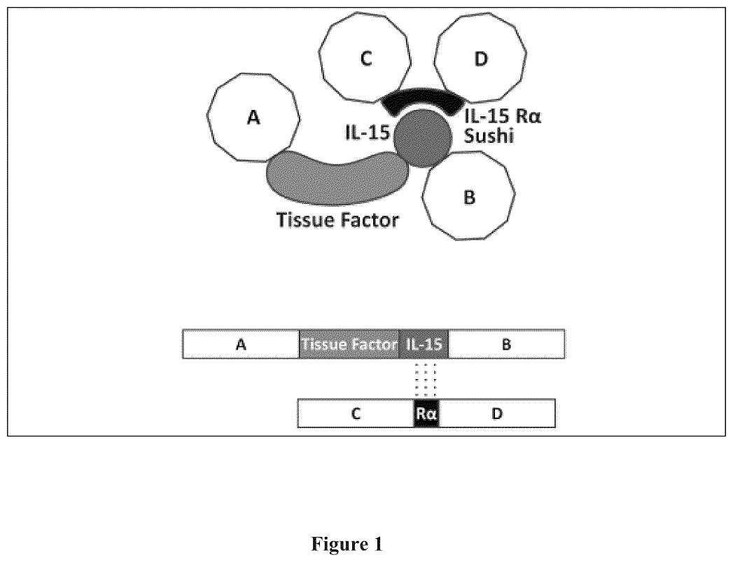

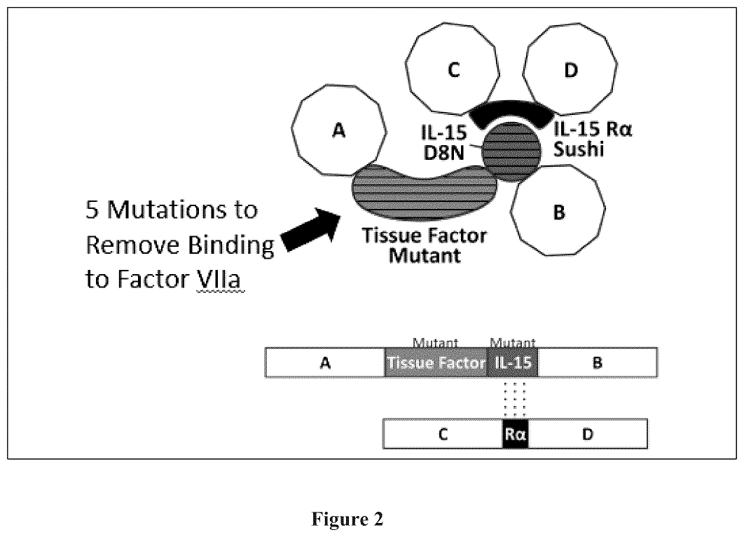

In some examples of any of the multi-chain chimeric polypeptides described herein the total length of first chimeric polypeptide and/or the second chimeric polypeptide can each independently be about 50 amino acids to about 3000 amino acids, about 100 amino acids to about 2000 amino acids, about 200 amino acids to about 1000 amino acids, about 300 amino acids to about 900 amino acids, about 400 amino acids to about 800 amino acids, about 500 amino acids to about 700 amino acids, or to about 600 amino acids. Diagrams of exemplary multi-chain chimeric polypeptides provided herein are depicted in FIGS. 1 and 2.

In some embodiments of any of the multi-chain chimeric polypeptides described herein, the first target-binding domain (e.g., any of the first target-binding domains described herein) and the soluble tissue factor domain (e.g., any of the exemplary soluble tissue factor domains described herein) directly abut each other in the first chimeric polypeptide. In some embodiments of any of the multi-chain chimeric polypeptides described herein, the first chimeric polypeptide further comprises a linker sequence (e.g., any of the exemplary linker sequences described herein or known in the art) between the first target-binding domain (e.g., any of the exemplary first target-binding domains described herein) and the soluble tissue factor domain (e.g., any of the exemplary soluble tissue factor domains described herein) in the first chimeric polypeptide.

In some embodiments of any of the multi-chain chimeric polypeptides described herein, the soluble tissue factor domain (e.g., any of the exemplary soluble tissue factor domains described herein) and the first domain of the pair of affinity domains (e.g., any of the exemplary first domains of any of the exemplary pairs of affinity domains described herein) directly abut each other in the first chimeric polypeptide. In some embodiments of any of the multi-chain chimeric polypeptides described herein, the first chimeric polypeptide further comprises a linker sequence (e.g., any of the exemplary linker sequences described herein or known in the art) between the soluble tissue factor domain (e.g., any of the exemplary soluble tissue factor domains described herein) and the first domain of the pair of affinity domains (e.g., any of the exemplary first domains of any of the exemplary pairs of affinity domains described herein) in the first chimeric polypeptide.

In some embodiments of any of the multi-chain chimeric polypeptides described herein, the second domain of the pair of affinity domains (e.g., any of the exemplary second domains of any of the exemplary pairs of affinity domains described herein) and the second target-binding domain (e.g., any of the exemplary second target-binding domains described herein) directly abut each other in the second chimeric polypeptide. In some embodiments of any of the multi-chain chimeric polypeptides described herein, the second chimeric polypeptide further comprises a linker sequence (e.g., any of the exemplary linker sequences described herein or known in the art) between the second domain of the pair of affinity domains (e.g., any of the exemplary second domains of any of the exemplary pairs of affinity domains described herein) and the second target-binding domain (e.g., any of the exemplary second target-binding domains described herein) in the second chimeric polypeptide.

Non-limiting aspects of these chimeric polypeptides, nucleic acids, vectors, cells, and methods are described below, and can be used in any combination without limitation. Additional aspects of these chimeric polypeptides, nucleic acids, vectors, cells, and methods are known in the art.

### Tissue Factor

Human tissue factor is a 263 amino-acid transmembrane protein containing three domains: (1) a 219-amino acid N-terminal extracellular domain (residues 1-219); (2) a 22-amino acid transmembrane domain (residues 220-242); and (3) a 21-amino acid cytoplasmic C-terminal tail (residues 242-263) ((UniProtKB Identifier Number: P13726). The cytoplasmic tail contains two phosphorylation sites at Ser253 and Ser258, and one S-palmitoylation site at Cys245. Deletion or mutation of the cytoplasmic domain was not found to affect tissue factor coagulation activity. Tissue factor has one S-palmitoylation site in the intracellular domain of the protein at Cys245. The Cys245 is located at the amino acid terminus of the intracellular domain and close to the membrane surface. The tissue factor transmembrane domain is composed of a single-spanning α-helix.

The extracellular domain of tissue factor, composed of two fibronectin type III domains, is connected to the transmembrane domain through a six-amino acid linker. This linker provides conformational flexibility to decouple the tissue factor extracellular domain from its transmembrane and cytoplasmic domains. Each tissue factor fibronectin type III module is composed of two overlapping β sheets with the top sheet domain containing three antiparallel β-strands and the bottom sheet containing four β-strands. The β-strands are connected by β-loops between strand βA and θB, βC and βD, and βE and βF, all of which are conserved in conformation in the two modules. There are three short α-helix segments connecting the β-strands. A unique feature of tissue factor is a 17-amino acid β-hairpin between strand β10 and strand β11, which is not a common element of the fibronectin superfamily. The N-terminal domain also contains a 12 amino acid loop between β6F and β7G that is not present in the C-terminal domain and is unique to tissue factor. Such a fibronectin type III domain structure is a feature of the immunoglobulin-like family of protein folds and is conserved among a wide variety of extracellular proteins.

The zymogen FVII is rapidly converted to FVIIa by limited proteolysis once it binds to tissue to form the active tissue factor-FVIIa complex. The FVIIa, which circulates as an enzyme at a concentration of approximately 0.1 nM (1% of plasma FVII), can also bind directly to tissue factor. The allosteric interaction between tissue factor and FVIIa on the tissue factor-FVIIa complex greatly increases the enzymatic activity of FVIIa: an approximate 20- to 100-fold increase in the rate of hydrolysis of small, chromogenic peptidyl substrates, and nearly a million-fold increase in the rate of activation of the natural macromolecular substrates FIX and FX. In concert with allosteric activation of the active site of FVIIa upon binding to tissue factor, the formation of tissue factor-FVIIa complex on phospholipid bilayer (i.e., upon exposure of phosphatidyl-L-serine on membrane surfaces) increases the rate of FIX or FX activation, in a Ca²⁺-dependent manner, an additional 1,000-fold. The roughly million-fold overall increase in FX activation by tissue factor-FVIIa-phospholipid complex relative to free FVIIa is a critical regulatory point for the coagulation cascade.

FVII is a ˜50 kDa, single-chain polypeptide consisting of 406 amino acid residues, with an N-terminal 7-carboxyglutamate-rich (GLA) domain, two epidermal growth factor-like domains (EGF1 and EFG2), and a C-terminal serine protease domain.

FVII is activated to FVIIa by a specific proteolytic cleavage of the Ile-¹⁵⁴-Arg¹⁵² bond in the short linker region between the EGF2 and the protease domain. This cleavage results in the light and heavy chains being held together by a single disulfide bond of Cys¹³⁵ and Cys²⁶². FVIIa binds phospholipid membrane in a Ca²⁺-dependent manner through its N-terminal GLA-domain. Immediately C-terminal to the GLA domain is an aromatic stack and two EGF domains. The aromatic stack connects the GLA to EGF1 domain which binds a single Ca²⁺ ion. Occupancy of this Ca²⁺-binding site increases FVIIa amidolytic activity and tissue factor association. The catalytic triad consist of His¹⁹³, Asp²⁴², and Ser³⁴⁴, and binding of a single Ca²⁺ ion within the FVIIa protease domain is critical for its catalytic activity. Proteolytic activation of FVII to FVIIa frees the newly formed amino terminus at Ile¹⁵³ to fold back and be inserted into the activation pocket forming a salt bridge with the carboxylate of Asp³⁴³ to generate the oxyanion hole. Formation of this salt bridge is critical for FVIIa activity. However, oxyanion hole formation does not occur in free FVIIa upon proteolytic activation. As a result, FVIIa circulates in a zymogen-like state that is poorly recognized by plasma protease inhibitors, allowing it to circulate with a half-life of approximately 90 minutes.

Tissue factor-mediated positioning of the FVIIa active site above the membrane surface is important for FVIIa towards cognate substrates. Free FVIIa adopts a stable, extended structure when bound to the membrane with its active site positioned ˜80 Å above the membrane surface. Upon FVIIa binding to tissue factor, the FVa active site is repositioned ˜6 Å closer to the membrane. This modulation may aid in a proper alignment of the FVIIa catalytic triad with the target substrate cleavage site. Using GLA-domainless FVIIa, it has been shown that the active site was still positioned a similar distance above the membrane, demonstrating that tissue factor is able to fully support FVIIa active site positioning even in the absence of FVIIa-membrane interaction. Additional data showed that tissue factor supported full FVIIa proteolytic activity as long as the tissue factor extracellular domain was tethered in some way to the membrane surface. However, raising the active site of FVIIa greater than 80 Å above the membrane surface greatly reduced the ability of the tissue factor-FVIIa complex to activate FX but did not diminish tissue factor-FVIIa amidolytic activity.

Alanine scanning mutagenesis has been used to assess the role of specific amino acid side chains in the tissue factor extracellular domain for interaction with FVIIa (Gibbs et al., Biochemistry 33(47): 14003-14010, 1994; Schullek et al., J Biol Chem 269(30): 19399-19403, 1994). Alanine substitution identified a limited number of residue positions at which alanine replacements cause 5- to 10-fold lower affinity for FVIIa binding. Most of these residue side chains were found to be well-exposed to solvent in the crystal structure, concordant with macromolecular ligand interaction. The FVIIa ligand-binding site is located over an extensive region at the boundary between the two modules. In the C-module, residues Arg¹³⁵ and Phe¹⁴⁰ located on the protruding B-C loop provide an independent contact with FVIIa. Leu¹³³ is located at the base of the fingerlike structure and packed into the cleft between the two modules. This provides continuity to a major cluster of important binding residues consisting of Lys²⁰, Thr⁶⁰, Asp⁵⁸, and Ile²². Thr⁶⁰ is only partially solvent-exposed and may play a local structural role rather than making a significant contact with ligand. The binding site extends onto the concave side of the intermodule angle involving Glu²⁴ and Gln¹¹⁰, and potentially the more distant residue Val²⁰⁷. The binding region extends from Asp58 onto a convex surface area formed by Lys⁴⁸, Lys⁴⁶, Gln³⁷, Asp⁴⁴, and Trp⁴⁵. Trp⁴⁵ and Asp⁴⁴ do not interact independently with FVIIa, indicating that the mutational effect at the Trp⁴⁵ position may reflect a structural importance of this side chain for the local packing of the adjacent Asp⁴⁴ and Gln³⁷ side chain. The interactive area further includes two surface-exposed aromatic residues, Phe⁷⁶ and Tyr⁷⁸, which form part of the hydrophobic cluster in the N-module.

The known physiologic substrates of tissue factor-FVIIa are FVII, FIX, and FX and certain proteinase-activated receptors. Mutational analysis has identified a number of residues that, when mutated, support full FVIIa amidolytic activity towards small peptidyl substrates but are deficient in their ability to support macromolecular substrate (i.e., FVII, FIX, and FX) activation (Ruf et al., J Biol Chem 267(31): 22206-22210, 1992; Ruf et al., J Biol Chem 267(9): 6375-6381, 1992; Huang et al., J Biol Chem 271(36): 21752-21757, 1996; Kirchhofer et al., Biochemistry 39(25): 7380-7387, 2000). The tissue factor loop region at residues 159-165, and residues in or adjacent to this flexible loop have been shown to be critical for the proteolytic activity of the tissue factor-FVIIa complex. This defines the proposed substrate-binding exosite region of tissue factor that is quite distant from the FVIIa active site. A substitution of the glycine residue by a marginally bulkier residue alanine, significantly impairs tissue factor-FVIIa proteolytic activity. This suggests that the flexibility afforded by glycine is critical for the loop of residues 159-165 for tissue factor macromolecular substrate recognition.

The residues Lys¹⁶⁵ and Lys¹⁶⁶ have also been demonstrated to be important for substrate recognition and binding. Mutation of either of these residues to alanine results in a significant decrease in the tissue factor co-factor function. Lys¹⁶⁵ and Lys¹⁶⁶ face away from each other, with Lys¹⁶⁵ pointing towards FVIIa in most tissue factor-FVIIa structures, and Lys¹⁶⁶ pointing into the substrate binding exosite region in the crystal structure. Putative salt bridge formation between Lys¹⁶⁵ of and Gla³⁵ of FVIIa would support the notion that tissue factor interaction with the GLA domain of FVIIa modulates substrate recognition. These results suggest that the C-terminal portion of the tissue factor ectodomain directly interacts with the GLA-domain, the possible adjacent EGF1 domains, of FIX and FX, and that the presence of the FVIIa GLA-domain may modulate these interactions either directly or indirectly.

### Soluble Tissue Factor Domain

In some embodiments of any of the polypeptides, compositions, or methods described herein, the soluble tissue factor domain can be a wildtype tissue factor polypeptide lacking the signal sequence, the transmembrane domain, and the intracellular domain. In some examples, the soluble tissue factor domain can be a tissue factor mutant, wherein a wildtype tissue factor polypeptide lacking the signal sequence, the transmembrane domain, and the intracellular domain, and has been further modified at selected amino acids. In some examples, the soluble tissue factor domain can be a soluble human tissue factor domain. In some examples, the soluble tissue factor domain can be a soluble mouse tissue factor domain. In some examples, the soluble tissue factor domain can be a soluble rat tissue factor domain. Non-limiting examples of soluble human tissue factor domains, a mouse soluble tissue factor domain, a rat soluble tissue factor domain, and mutant soluble tissue factor domains are shown below.

Exemplary Soluble Human Tissue Factor Domain
(SEQ ID NO: 1)
SGTTNTVAAYNLTWKSTNFKTILEWEPKPVNQVYTVQISTKSGDW
KSKCFYTTDTECDLTDEIVKDVKQTYLARVFSYPAGNVESTGSAG
EPLYENSPEFTPYLETNLGQPTIQSFEQVGTKVNVTVEDERTLVR
RNNTFLSLRDVFGKDLIYTLYYWKSSSSGKKTAKTNTNEFLIDVD
KGENYCFSVQAVIPSRTVNRKSTDSPVECMGQEKGEFRE
Exemplary Nucleic Acid Encoding Soluble
Human Tissue Factor Domain
(SEQ ID NO: 9)
AGCGGCACAACCAACACAGTCGCTGCCTATAACCTCACTTGGAAG
AGCACCAACTTCAAAACCATCCTCGAATGGGAACCCAAACCCGTT
AACCAAGTTTACACCGTGCAGATCAGCACCAAGTCCGGCGACTGG
AAGTCCAAATGTTTCTATACCACCGACACCGAGTGCGATCTCACC
GATGAGATCGTGAAAGATGTGAAACAGACCTACCTCGCCCGGGTG
TTTAGCTACCCCGCCGGCAATGTGGAGAGCACTGGTTCCGCTGGC
GAGCCTTTATACGAGAACAGCCCCGAATTTACCCCTTACCTCGAG
ACCAATTTAGGACAGCCCACCATCCAAAGCTTTGAGCAAGTTGGC
ACAAAGGTGAATGTGACAGTGGAGGACGAGCGGACTTTAGTGCGG
CGGAACAACACCTTTCTCAGCCTCCGGGATGTGTTCGGCAAAGAT
TTAATCTACACACTGTATTACTGGAAGTCCTCTTCCTCCGGCAAG
AAGACAGCTAAAACCAACACAAACGAGTTTTTAATCGACGTGGAT
AAAGGCGAAAACTACTGTTTCAGCGTGCAAGCTGTGATCCCCTCC
CGGACCGTGAATAGGAAAAGCACCGATAGCCCCGTTGAGTGCATG
GGCCAAGAAAAGGGCGAGTTCCGGGAG

In some embodiments, a soluble tissue factor domain can include a sequence that is at least 70% identical, at least 72% identical, at least 74% identical, at least 76% identical, at least 78% identical, at least 80% identical, at least 82% identical, at least 84% identical, at least 86% identical, at least 88% identical, at least 90% identical, at least 92% identical, at least 94% identical, at least 96% identical, at least 98% identical, at least 99% identical, or 100% identical to SEQ ID NO:1. In some embodiments, a soluble tissue factor domain can include a sequence of SEQ ID NO:1, with one to twenty amino acids (e.g., 2, 3, 4, 5, 6, 7, 8, 9, 10, 11, 12, 13, 14, 15, 16, 17, 18, 19, or 20) amino acids removed from its N-terminus and/or one to twenty amino acids (e.g., 2, 3, 4, 5, 6, 7, 8, 9, 10, 11, 12, 13, 14, 15, 16, 17, 18, 19, or 20) amino acids removed from its C-terminus.

As can be appreciated in the art, one skilled in the art would understand that mutation of amino acids that are conserved between different mammalian species is more likely to decrease the activity and/or structural stability of the protein, while mutation of amino acids that are not conserved between different mammalian species is less likely to decrease the activity and/or structural stability of the protein.

In some examples of any of the multi-chain chimeric polypeptides described herein, the soluble tissue factor domain is not capable of binding to Factor VIIa. In some examples of any of the multi-chain chimeric polypeptides described herein, the soluble tissue factor domain does not convert inactive Factor X into Factor Xa. In some embodiments of any of the multi-chain chimeric polypeptides described herein, the multi-chain chimeric polypeptide does not stimulate blood coagulation in a mammal.

In some examples, the soluble tissue factor domain can be a soluble human tissue factor domain. In some embodiments, the soluble tissue factor domain can be a soluble mouse tissue factor domain. In some embodiments, the soluble tissue factor domain can be a soluble rat tissue factor domain.

In some examples, the soluble tissue factor domain does not include one or more (e.g., two, three, four, five, six, or seven) of: a lysine at an amino acid position that corresponds to amino acid position 20 of mature wildtype human tissue factor protein; an isoleucine at an amino acid position that corresponds to amino acid position 22 of mature wildtype human tissue factor protein; a tryptophan at an amino acid position that corresponds to amino acid position 45 of mature wildtype human tissue factor protein; an aspartic acid at an amino acid position that corresponds to amino acid position 58 of mature wildtype human tissue factor protein; a tyrosine at an amino acid position that corresponds to amino acid position 94 of mature wildtype human tissue factor protein; an arginine at an amino acid position that corresponds to amino acid position 135 of mature wildtype human tissue factor protein; and a phenylalanine at an amino acid position that corresponds to amino acid position 140 of mature wildtype human tissue factor protein.

In some examples, the soluble tissue factor domain can be encoded by a nucleic acid including a sequence that is at least 70% identical, at least 72% identical, at least 74% identical, at least 76% identical, at least 78% identical, at least 80% identical, at least 82% identical, at least 84% identical, at least 86% identical, at least 88% identical, at least 90% identical, at least 92% identical, at least 94% identical, at least 96% identical, at least 98% identical, at least 99% identical, or 100% identical to SEQ ID NO: 9.

In some embodiments, the soluble tissue factor domain can have a total length of about 20 amino acids to about 220 amino acids, about 30 amino acids to about 200 amino acids, about 40 amino acids to about 180 amino acids, about 50 amino acids to about 160 amino acids, about 60 amino acids to about 140 amino acids, about 70 amino acids to about 120 amino acids, about 80 amino acids to about 110 amino acids, or about 90 amino acids to about 100 amino acids.

### Linker Sequences

In some embodiments, the linker sequence can be a flexible linker sequence. Non-limiting examples of linker sequences that can be used are described in Klein et al., Protein Engineering, Design & Selection 27(10):325-330, 2014; Priyanka et al., Protein Sci. 22(2):153-167, 2013. In some examples, the linker sequence is a synthetic linker sequence.

In some embodiments of any of the multi-chain chimeric polypeptides described herein, the first chimeric polypeptide can include one, two, three, four, five, six, seven, eight, nine, or ten linker sequence(s) (e.g., the same or different linker sequences, e.g., any of the exemplary linker sequences described herein or known in the art). In some embodiments of any of the multi-chain chimeric polypeptides described herein, the second chimeric polypeptide can include one, two, three, four, five, six, seven, eight, nine, or ten linker sequence(s) (e.g., the same or different linker sequences, e.g., any of the exemplary linker sequences described herein or known in the art).

In some embodiments, a linker sequence can have a total length of 1 amino acid to about 100 amino acids, about 2 amino acids to about 90 amino acids, about 4 amino acids to about 80 amino acids, about 6 amino acids to about 70 amino acids, about 8 amino acids to about 60 amino acids, about 10 amino acids to about 50 amino acids, about 12 amino acids to about 40 amino acids, about 14 amino acids to about 30 amino acids, or about 16 amino acids to about 20 amino acids.

In some embodiments, the linker is rich in glycine (Gly or G) residues. In some embodiments, the linker is rich in serine (Ser or S) residues. In some embodiments, the linker is rich in glycine and serine residues. In some embodiments, the linker has one or more glycine-serine residue pairs (GS), e.g., 1, 2, 3, 4, 5, 6, 7, 8, 9, or 10 or more GS pairs. In some embodiments, the linker has one or more Gly-Gly-Gly-Ser (GGGS) sequences, e.g., 1, 2, 3, 4, 5, 6, 7, 8, 9, or 10 or more GGGS sequences. In some embodiments, the linker has one or more Gly-Gly-Gly-Gly-Ser (GGGGS) sequences, e.g., 1, 2, 3, 4, 5, 6, 7, 8, 9, or 10 or more GGGGS sequences. In some embodiments, the linker has one or more Gly-Gly-Ser-Gly (GGSG) sequences, e.g., 1, 2, 3, 4, 5, 6, 7, 8, 9, or 10 or more GGSG sequences.

In some embodiments, the linker sequence can comprise or consist of GGGGSGGGGSGGGGS (SEQ ID NO:3). In some embodiments, the linker sequence can be encoded by a nucleic acid comprising or consisting of: GGCGGTGGAGGATCCGGAGGAGGTGGCTCCGGCGGCGGAGGATCT (SEQ ID NO:14). In some embodiments, the linker sequence can comprise or consist of: GGGSGGGS.

### Target-Binding Domains

In some embodiments of any of the multi-chain chimeric polypeptides described herein, the first target-binding domain, the second target-binding domain, and/or the additional one or more target-binding domains can be an antigen-binding domain that binds specifically to a ligand of TGF-βRII (e.g., any of the exemplary antigen-binding domains described herein or known in the art) or a soluble interleukin or cytokine receptor that binds specifically to a ligand of TGF-βRII (e.g., any of the exemplary soluble interleukin receptors or soluble cytokine receptors described herein).

Any of the target-binding domains described herein can bind to a ligand of TGF-βRII with a dissociation equilibrium constant (K_{D}) of less than 1×10⁻⁷ M, less than 1×10⁻⁸ M, less than 1×10⁻⁹ M, less than 1×10⁻¹⁰ M, less than 1×10⁻¹¹ M, less than 1×10⁻¹² M, or less than 1×10⁻¹³ M. In some embodiments, the antigen-binding protein construct provided herein can bind to an identifying antigen with a K_{D} of about 1×10⁻³ M to about 1×10⁻⁵ M, about 1×10⁻⁴ M to about 1×10⁻⁶ M, about 1×10⁻⁵ M to about 1×10⁻⁷ M, about 1×10⁻⁶ M to about 1×10⁻⁸ M, about 1×10⁻⁷ M to about 1×10⁻⁹ M, about 1×10⁻⁸ M to about 1×10⁻¹⁰ M, or about 1×10⁻⁹ M to about 1×10⁻¹¹ M (inclusive).

Any of the target-binding domains described herein can bind to a ligand of TGF-βRII (e.g., TGF-β) with a K_{D} of between about 1 pM to about 30 nM.

Any of the target-binding domains described herein can bind to a ligand of TGFβRII with a K_{D} of between about 1 nM to about 10 nM.

A variety of different methods known in the art can be used to determine the K_{D} values of any of the antigen-binding protein constructs described herein (e.g., an electrophoretic mobility shift assay, a filter binding assay, surface plasmon resonance, and a biomolecular binding kinetics assay, etc.).

### Antigen-Binding Domains

In some embodiments of any of the multi-chain chimeric polypeptides described herein, the first target-binding domain and the second target-binding domain bind specifically to the same antigen. In some embodiments of these multi-chain chimeric polypeptides, the first target-binding domain and the second target-binding domain bind specifically to the same epitope. In some embodiments of these multi-chain chimeric polypeptides, the first target-binding domain and the second target-binding domain include the same amino acid sequence.

In some embodiments of any of the multi-chain chimeric polypeptides described herein, the first target-binding domain and the second target-binding domain bind specifically to different antigens.

In some embodiments of any of the multi-chain chimeric polypeptides described herein, one or both of the first target-binding domain and the second target-binding domain is an antigen-binding domain. In some embodiments of any of the multi-chain chimeric polypeptides described herein, the first target-binding domain and the second target-binding domain are each antigen-binding domains.

In some embodiments of any of the multi-chain chimeric polypeptides described herein, the antigen-binding domain includes or is a scFv or a single domain antibody (e.g., a VHH or a VNAR domain).

In some examples, an antigen-binding domain (e.g., any of the antigen-binding domains described herein) can bind specifically to a ligand of TGF-βRII (see, e.g., antigen-binding domains that can bind specifically to TGF-β described in US 2021/0061897, US 2020/0399358, US 2020/0392221, US 2019/0315850, and US 2019/0177406, each of which is herein incorporated by reference).

The antigen-binding domains present in any of the multi-chain chimeric polypeptides described herein are each independently selected from the group consisting of: a VHH domain, a VNAR domain, and a scFv. In some embodiments, any of the antigen-binding domains described herein is a BiTe, a (scFv)₂, a nanobody, a nanobody-HSA, a DART, a TandAb, a scDiabody, a scDiabody-CH3, scFv-CH-CL-scFv, a HSAbody, scDiabody-HAS, or a tandem-scFv. Additional examples of antigen-binding domains that can be used in any of the multi-chain chimeric polypeptide are known in the art.

A VHH domain is a single monomeric variable antibody domain that can be found in camelids. A VNAR domain is a single monomeric variable antibody domain that can be found in cartilaginous fish. Non-limiting aspects of VHH domains and VNAR domains are described in, e.g., Cromie et al., Curr. Top. Med. Chem. 15:2543-2557, 2016; De Genst et al., Dev. Comp. Immunol. 30:187-198, 2006; De Meyer et al., Trends Biotechnol. 32:263-270, 2014; Kijanka et al., Nanomedicine 10:161-174, 2015; Kovaleva et al., Expert. Opin. Biol. Ther. 14:1527-1539, 2014; Krah et al., Immunopharmacol. Immunotoxicol. 38:21-28, 2016; Mujic-Delic et al., Trends Pharmacol. Sci. 35:247-255, 2014; Muyldermans, J. Biotechnol. 74:277-302, 2001; Muyldermans et al., Trends Biochem. Sci. 26:230-235, 2001; Muyldermans, Ann. Rev. Biochem. 82:775-797, 2013; Rahbarizadeh et al., Immunol. Invest. 40:299-338, 2011; Van Audenhove et al., EBioMedicine 8:40-48, 2016; Van Bockstaele et al., Curr. Opin. Investig. Drugs 10:1212-1224, 2009; Vincke et al., Methods Mol. Biol. 911:15-26, 2012; and Wesolowski et al., Med. Microbiol. Immunol. 198:157-174, 2009.

In some embodiments, each of the antigen-binding domains in the multi-chain chimeric polypeptides described herein are both VHH domains, or at least one antigen-binding domain is a VHH domain. In some embodiments, each of the antigen-binding domains in the multi-chain chimeric polypeptides described herein are both VNAR domains, or at least one antigen-binding domain is a VNAR domain. In some embodiments, each of the antigen-binding domains in the multi-chain chimeric polypeptides described herein are both scFv domains, or at least one antigen-binding domain is a scFv domain.

In some embodiments, two or more of polypeptides present in the multi-chain chimeric polypeptide can assemble (e.g., non-covalently assemble) to form any of the antigen-binding domains described herein, e.g., an antigen-binding fragment of an antibody (e.g., any of the antigen-binding fragments of an antibody described herein), a VHH-scAb, a VHH-Fab, a Dual scFab, a F(ab′)2, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a κλ-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)—IgG, IgG(L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)2-scFv2, a scFv-KIH, a Fab-scFv-Fc, a tetravalent HCAb, a scDiabody-Fc, a Diabody-Fc, a tandem scFv-Fc, an Intrabody, a dock and lock, a lmmTAC, an IgG-IgG conjugate, a Cov-X-Body, and a scFv1-PEG-scFv2. See, e.g., Spiess et al., Mol. Immunol. 67:95-106, 2015, incorporated in its entirety herewith, for a description of these elements. Non-limiting examples of an antigen-binding fragment of an antibody include an Fv fragment, a Fab fragment, a F(ab′)2 fragment, and a Fab′ fragment. Additional examples of an antigen-binding fragment of an antibody is an antigen-binding fragment of an IgG (e.g., an antigen-binding fragment of IgG1, IgG2, IgG3, or IgG4) (e.g., an antigen-binding fragment of a human or humanized IgG, e.g., human or humanized IgG1, IgG2, IgG3, or IgG4); an antigen-binding fragment of an IgA (e.g., an antigen-binding fragment of IgA1 or IgA2) (e.g., an antigen-binding fragment of a human or humanized IgA, e.g., a human or humanized IgA1 or IgA2); an antigen-binding fragment of an IgD (e.g., an antigen-binding fragment of a human or humanized IgD); an antigen-binding fragment of an IgE (e.g., an antigen-binding fragment of a human or humanized IgE); or an antigen-binding fragment of an IgM (e.g., an antigen-binding fragment of a human or humanized IgM).

An “Fv” fragment includes a non-covalently-linked dimer of one heavy chain variable domain and one light chain variable domain.

A “Fab” fragment includes, the constant domain of the light chain and the first constant domain (C_{H1}) of the heavy chain, in addition to the heavy and light chain variable domains of the Fv fragment.

A “F(ab′)2” fragment includes two Fab fragments joined, near the hinge region, by disulfide bonds.

A “dual variable domain immunoglobulin” or “DVD-Ig” refers to multivalent and multispecific binding proteins as described, e.g., in DiGiammarino et al., Methods Mol. Biol. 899:145-156, 2012; Jakob et al., MABs 5:358-363, 2013; and U.S. Pat. Nos. 7,612,181; 8,258,268; 8,586,714; 8,716,450; 8,722,855; 8,735,546; and 8,822,645, each of which is incorporated by reference in its entirety.

DARTs are described in, e.g., Garber, Nature Reviews Drug Discovery 13:799-801, 2014.

In some embodiments of any of the antigen-binding domains described herein can bind to an antigen selected from the group consisting of: a protein, a carbohydrate, a lipid, and a combination thereof.

Additional examples and aspects of antigen-binding domains are known in the art.

### Soluble Receptor

In some embodiments of any of the multi-chain chimeric polypeptides described herein, one or both of the first target-binding domain and the second target-binding domain is a soluble receptor. In some embodiments, the soluble receptor is a soluble TGF-β receptor II (TGF-β RII) (see, e.g., those described in Yung et al., Am. J. Resp. Crit. Care Med. 194(9):1140-1151, 2016) or a soluble TGF-βRIII (see, e.g., those described in Heng et al., Placenta 57:320, 2017).

Additional examples of soluble receptors are known in the art. Exemplary soluble receptors are described herein.

### Additional Target-Binding Domains

In some embodiments of any of the multi-chain chimeric polypeptides, the first chimeric polypeptide further includes one or more (e.g., two, three, four, five, six, seven, eight, nine, or ten) additional target-binding domain(s) (e.g., any of the exemplary target-binding domains described herein or known in the art), where at least one of the one or more additional antigen-binding domain(s) is positioned between the soluble tissue factor domain (e.g., any of the exemplary soluble tissue factor domains described herein or known in the art) and the first domain of the pair of affinity domains (e.g., any of the exemplary first domains of any of the exemplary pairs of affinity domains described herein). In some embodiments, the first chimeric polypeptide can further include a linker sequence (e.g., any of the exemplary linker sequences described herein or known in the art) between the soluble tissue factor domain (e.g., any of the exemplary soluble tissue factor domains described herein) and the at least one of the one or more additional target-binding domain(s) (e.g., any of the exemplary target-binding domains described herein or known in the art), and/or a linker sequence (e.g., any of the exemplary linker sequences described herein or known in the art) between the at least one of the one or more additional target-binding domain(s) (e.g., any of the exemplary target-binding domains described herein or known in the art) and the first domain of the pair of affinity domains (e.g., any of the exemplary first domains described herein of any of the exemplary pairs of affinity domains described herein).

In some embodiments of any of the multi-chain chimeric polypeptides described herein, the first chimeric polypeptide further includes one or more (e.g., two, three, four, five, six, seven, eight, nine, or ten) additional target-binding domains at the N-terminal and/or C-terminal end of the first chimeric polypeptide. In some embodiments, at least one of the one or more additional target-binding domains (e.g., any of the exemplary target-binding domains described herein or known in the art) directly abuts the first domain of the pair of affinity domains (e.g., any of the exemplary first domains described herein of any of the exemplary pairs of affinity domains described herein) in the first chimeric polypeptide. In some embodiments, the first chimeric polypeptide further includes a linker sequence (e.g., any of the exemplary linker sequences described herein or known in the art) between the at least one of the one or more additional target-binding domains (e.g., any of the exemplary target-binding domains described herein or known in the art) and the first domain of the pair of affinity domains (e.g., any of the exemplary first domains described herein of any of the exemplary pairs of affinity domains described herein). In some embodiments, the at least one of the one or more additional target-binding domains (e.g., any of the exemplary target-binding domains described herein or known in the art) directly abuts the first target-binding domain (e.g., any of the exemplary target-binding domains described herein or known in the art) in the first chimeric polypeptide. In some embodiments, the first chimeric polypeptide further comprises a linker sequence (e.g., any of the exemplary linker sequences described herein or known in the art) between the at least one of the one or more additional target-binding domains (e.g., any of the exemplary target-binding domains described herein or known in the art) and the first target-binding domain (e.g., any of the exemplary target-binding domains described herein or known in the art).

In some embodiments of any of the multi-chain chimeric polypeptides described herein, at least one of the one or more additional target-binding domains (e.g., any of the exemplary target-binding domains described herein or known in the art) is disposed at the N- and/or C-terminus of the first chimeric polypeptide, and at least one of the one or more additional target-binding domains (e.g., any of the exemplary target-binding domains described herein or known in the art) is positioned between the soluble tissue factor domain (e.g., any of the exemplary soluble tissue factor domains described herein or known in the art) and the first domain of the pair of affinity domains (e.g., any of the exemplary first domains of any of the exemplary pairs of affinity domains described herein) in the first chimeric polypeptide. In some embodiments, the at least one additional target-binding domain (e.g., any of the exemplary target-binding domains described herein or known in the art) of the one or more additional target-binding domains disposed at the N-terminus directly abuts the first target-binding domain (e.g., any of the exemplary target-binding domains described herein or known in the art) or the first domain of the pair of affinity domains (e.g., any of the exemplary first domains described herein of any of the exemplary pairs of affinity domains described herein) in the first chimeric polypeptide. In some embodiments, the first chimeric polypeptide further comprises a linker sequence (e.g., any of the linker sequences described herein or known in the art) disposed between the at least one additional target-binding domain (e.g., any of the exemplary target-binding domains described herein or known in the art) and the first target-binding domain (e.g., any of the exemplary target-binding domains described herein or known in the art) or the first domain of the pair of affinity domains (e.g., any of the exemplary first domains described herein of any of the exemplary pairs of affinity domains described herein) in the first chimeric polypeptide. In some embodiments, the at least one additional target-binding domain (e.g., any of the exemplary target-binding domains described herein or known in the art) of the one or more additional target-binding domains disposed at the C-terminus directly abuts the first target-binding domain (e.g., any of the exemplary target-binding domains described herein or known in the art) or the first domain of the pair of affinity domains (e.g., any of the exemplary first domains of any of the exemplary pairs of affinity domains described herein) in the first chimeric polypeptide. In some embodiments, the first chimeric polypeptide further includes a linker sequence (e.g., any of the exemplary linker sequences described herein or known in the art) disposed between the at least one additional target-binding domain (e.g., any of the exemplary target-binding domains described herein or known in the art) and the first target-binding domain (e.g., any of the exemplary target-binding domains described herein or known in the art) or the first domain of the pair of affinity domains (e.g., any of the exemplary first domains described herein of any of the exemplary pairs of affinity domains described herein) in the first chimeric polypeptide. In some embodiments, the at least one of the one or more additional target-binding domains (e.g., any of the exemplary target-binding domains described herein or known in the art) positioned between the soluble tissue factor domain (e.g., any of the exemplary soluble tissue factor domains described herein) and the first domain of the pair of affinity domains (e.g., any of the first domains described herein or any of the exemplary pairs of affinity domains described herein), directly abuts the soluble tissue factor domain and/or the first domain of the pair of affinity domains. In some embodiments, the first chimeric polypeptide further comprises a linker sequence (e.g., any of the exemplary linker sequences described herein or known in the art) disposed (i) between the soluble tissue factor domain (e.g., any of the exemplary soluble tissue factor domains described herein) and the at least one of the one or more additional target-binding domains (e.g., any of the exemplary target-binding domains described herein or known in the art) positioned between the soluble tissue factor domain (e.g., any of the exemplary soluble tissue factor domains described herein) and the first domain of the pair of affinity domains (e.g., any of the exemplary first domains of any of the exemplary pairs of affinity domains described herein), and/or (ii) between the first domain of the pair of affinity domains and the at least one of the one or more additional target-binding domains positioned between the soluble tissue factor domain and the first domain of the pair of affinity domains.

In some embodiments of any of the multi-chain chimeric polypeptides described herein, the second chimeric polypeptide further includes one or more (e.g., two, three, four, five, six, seven, eight, nine, or ten) additional target-binding domains (e.g., any of the exemplary target-binding domains described herein or known in the art) at the N-terminal end and/or the C-terminal end of the second chimeric polypeptide. In some embodiments, at least one of the one or more additional target-binding domains (e.g., any of the exemplary target-binding domains described herein or known in the art) directly abuts the second domain of the pair of affinity domains (e.g., any of the exemplary second domains of any of the exemplary pairs of affinity domains described herein) in the second chimeric polypeptide. In some embodiments, the second chimeric polypeptide further includes a linker sequence (e.g., any of the exemplary linker sequences described herein or known in the art) between at least one of the one or more additional target-binding domains (e.g., any of the exemplary target-binding domains described herein or known in the art) and the second domain of the pair of affinity domains (e.g., any of the second domains described herein of any of the exemplary pairs of affinity domains described herein) in the second chimeric polypeptide. In some embodiments, at least one of the one or more additional target-binding domains (e.g., any of the exemplary target-binding domains described herein or known in the art) directly abuts the second target-binding domain (e.g., any of the target-binding domains described herein or known in the art) in the second chimeric polypeptide. In some embodiments, the second chimeric polypeptide further includes a linker sequence (e.g., any of the exemplary linker sequences described herein or known in the art) between at least one of the one or more additional target-binding domains (e.g., any of the exemplary target binding domains described herein or known in the art) and the second target-binding domain (e.g., any of the exemplary target binding domains described herein or known in the art) in the second chimeric polypeptide.

In some embodiments of any of the multi-chain chimeric polypeptides described herein, two or more (e.g., three or more, four or more, five or more, six or more, seven or more, eight or more, nine or more, or ten or more) of the first target-binding domain, the second target-binding domain, and the one or more additional target-binding domains bind specifically to the same antigen. In some embodiments, two or more (e.g., three or more, four or more, five or more, six or more, seven or more, eight or more, nine or more, or ten or more) of the first target-binding domain, the second target-binding domain, and the one or more additional target-binding domains bind specifically to the same epitope. In some embodiments, two or more (e.g., three or more, four or more, five or more, six or more, seven or more, eight or more, nine or more, or ten or more) of the first target-binding domain, the second target-binding domain, and the one or more additional target-binding domains include the same amino acid sequence. In some embodiments, the first target-binding domain, the second target-binding domain, and the one or more additional target-binding domains each bind specifically to the same antigen. In some embodiments, the first target-binding domain, the second target-binding domain, and the one or more additional target-binding domains each bind specifically to the same epitope. In some embodiments, the first target-binding domain, the second target-binding domain, and the one or more additional target-binding domains each include the same amino acid sequence.

In some embodiments of any of the multi-chain chimeric polypeptides described herein, the first target-binding domain, the second target-binding domain, and the one or more additional target-binding domains bind specifically to different antigens. In some embodiments of any of the multi-chain chimeric polypeptides described herein, one or more (e.g., two or more, three or more, four or more, five or more, six or more, seven or more, eight or more, nine or more, or ten or more) of the first target-binding domain, the second target-binding domain, and the one or more target-binding domains is an antigen-binding domain. In some embodiments, the first target-binding domain, the second target-binding domain, and the one or more additional target-binding domains are each an antigen-binding domain (e.g., a scFv or a single-domain antibody).

### Pairs of Affinity Domains

In some embodiments, a multi-chain chimeric polypeptide includes: 1) a first chimeric polypeptide that includes a first domain of a pair of affinity domains, and 2) a second chimeric polypeptide that includes a second domain of a pair of affinity domains such that the first chimeric polypeptide and the second chimeric polypeptide associate through the binding of the first domain and the second domain of the pair of affinity domains. In some embodiments, the pair of affinity domains is a sushi domain from an alpha chain of human IL-15 receptor (IL15Rα) and a soluble IL-15. A sushi domain, also known as a short consensus repeat or type 1 glycoprotein motif, is a common motif in protein-protein interaction. Sushi domains have been identified on a number of protein-binding molecules, including complement components C1r, C1s, factor H, and C2m, as well as the nonimmunologic molecules factor XIII and β2-glycoprotein. A typical Sushi domain has approximately 60 amino acid residues and contains four cysteines (Ranganathan, Pac. Symp Biocomput. 2000:155-67). The first cysteine can form a disulfide bond with the third cysteine, and the second cysteine can form a disulfide bridge with the fourth cysteine. In some embodiments in which one member of the pair of affinity domains is a soluble IL-15, the soluble IL15 has a D8N or D8A amino acid substitution. In some embodiments in which one member of the pair of affinity domains is an alpha chain of human IL-15 receptor (IL115Rα), the human IL15Rα is a mature full-length IL15Rα. In some embodiments, the pair of affinity domains is barnase and barnstar. In some embodiments, the pair of affinity domains is a PKA and an AKAP. In some embodiments, the pair of affinity domains is an adapter/docking tag module based on mutated RNase I fragments (Rossi, Proc Natl Acad Sci USA. 103:6841-6846, 2006; Sharkey et al., Cancer Res. 68:5282-5290, 2008; Rossi et al., Trends Pharmacol Sci. 33:474-481, 2012) or SNARE modules based on interactions of the proteins syntaxin, synaptotagmin, synaptobrevin, and SNAP25 (Deyev et al., Nat Biotechnol. 1486-1492, 2003).

In some embodiments, a first chimeric polypeptide of a multi-chain chimeric polypeptide includes a first domain of a pair of affinity domains and a second chimeric polypeptide of the multi-chain chimeric polypeptide includes a second domain of a pair of affinity domains, wherein the first domain of the pair of affinity domains and the second domain of the pair of affinity domains bind to each other with a dissociation equilibrium constant (K_{D}) of less than 1×10⁻⁷ M, less than 1×10⁻⁸ M, less than 1×10⁻⁹ M, less than 1×10⁻¹⁰ M, less than 1×10⁻¹¹ M, less than 1×10⁻¹² M, or less than 1×10⁻¹³ M. In some embodiments, the first domain of the pair of affinity domains and the second domain of the pair of affinity domains bind to each other with a K_{D} of about 1×10⁻⁴ M to about 1×10⁻⁶ M, about 1×10⁻⁵ M to about 1×10⁻⁷ M, about 1×10⁻⁶ M to about 1×10⁻⁸ M, about 1×10⁻⁷ M to about 1×10⁻⁹ M, about 1×10⁻⁸ M to about 1×10⁻¹⁰ M, about 1×10⁻⁹ M to about 1×10⁻¹¹ M, about 1×10⁻¹⁰ M to about 1×10⁻¹² M, about 1×10⁻¹¹ M to about 1×10⁻¹³ M, about 1×10⁻⁴ M to about 1×10⁻⁵ M, about 1×10⁻⁵ M to about 1×10⁻⁶ M, about 1×10⁻⁶ M to about 1×10⁻⁷ M, about 1×10⁻⁷ M to about 1×10⁻⁸ M, about 1×10⁻⁸ M to about 1×10⁻⁹ M, about 1×10⁻⁹ M to about 1×10⁻¹⁰ M, about 1×10⁻¹⁰ M to about 1×10⁻¹¹ M, about 1×10⁻¹¹ M to about 1×10⁻¹² M, or about 1×10⁻¹² M to about 1×10⁻¹³ M (inclusive). Any of a variety of different methods known in the art can be used to determine the K_{D} value of the binding of the first domain of the pair of affinity domains and the second domain of the pair of affinity domains (e.g., an electrophoretic mobility shift assay, a filter binding assay, surface plasmon resonance, and a biomolecular binding kinetics assay, etc.).

In some embodiments, a first chimeric polypeptide of a multi-chain chimeric polypeptide includes a first domain of a pair of affinity domains and a second chimeric polypeptide of the multi-chain chimeric polypeptide includes a second domain of a pair of affinity domains, wherein the first domain of the pair of affinity domains, the second domain of the pair of affinity domains, or both is about 10 to 100 amino acids in length. For example, a first domain of a pair of affinity domains, a second domain of a pair of affinity domains, or both can be about 10 to 100 amino acids in length, about 15 to 100 amino acids in length, about 20 to 100 amino acids in length, about 25 to 100 amino acids in length, about 30 to 100 amino acids in length, about 35 to 100 amino acids in length, about 40 to 100 amino acids in length, about 45 to 100 amino acids in length, about 50 to 100 amino acids in length, about 55 to 100 amino acids in length, about 60 to 100 amino acids in length, about 65 to 100 amino acids in length, about 70 to 100 amino acids in length, about 75 to 100 amino acids in length, about 80 to 100 amino acids in length, about 85 to 100 amino acids in length, about 90 to 100 amino acids in length, about 95 to 100 amino acids in length, about 10 to 95 amino acids in length, about 10 to 90 amino acids in length, about 10 to 85 amino acids in length, about 10 to 80 amino acids in length, about 10 to 75 amino acids in length, about 10 to 70 amino acids in length, about 10 to 65 amino acids in length, about 10 to 60 amino acids in length, about 10 to 55 amino acids in length, about 10 to 50 amino acids in length, about 10 to 45 amino acids in length, about 10 to 40 amino acids in length, about 10 to 35 amino acids in length, about 10 to 30 amino acids in length, about 10 to 25 amino acids in length, about 10 to 20 amino acids in length, about 10 to 15 amino acids in length, about 20 to 30 amino acids in length, about 30 to 40 amino acids in length, about 40 to 50 amino acids in length, about 50 to 60 amino acids in length, about 60 to 70 amino acids in length, about 70 to 80 amino acids in length, about 80 to 90 amino acids in length, about 90 to 100 amino acids in length, about 20 to 90 amino acids in length, about 30 to 80 amino acids in length, about 40 to 70 amino acids in length, about 50 to 60 amino acids in length, or any range in between. In some embodiments, a first domain of a pair of affinity domains, a second domain of a pair of affinity domains, or both is about 10, 15, 20, 25, 30, 35, 40, 45, 50, 55, 60, 65, 70, 75, 80, 85, 90, 95, or 100 amino acids in length.

In some embodiments, any of the first and/or second domains of a pair of affinity domains disclosed herein can include one or more additional amino acids (e.g., 1, 2, 3, 5, 6, 7, 8, 9, 10, or more amino acids) at its N-terminus and/or C-terminus, so long as the function of the first and/or second domains of a pair of affinity domains remains intact. For example, a sushi domain from an alpha chain of human IL-15 receptor (IL15Rα) can include one or more additional amino acids at the N-terminus and/or the C-terminus, while still retaining the ability to bind to a soluble IL-15. Additionally or alternatively, a soluble IL-15 can include one or more additional amino acids at the N-terminus and/or the C-terminus, while still retaining the ability to bind to a sushi domain from an alpha chain of human IL-15 receptor (IL15Rα).

A non-limiting example of a sushi domain from an alpha chain of IL-15 receptor alpha (IL15Rα) can include a sequence that is at least 70% identical, at least 75% identical, at least 80% identical, at least 85% identical, at least 90% identical, at least 95% identical, at least 99% identical, or 100% identical to ITCPPPMSVEHADIWVKSYSLYSRERYICNSGFKRKAGTSSLTECVLNKATNVAH WTTPSLKCIR (SEQ ID NO:11). In some embodiments, a sushi domain from an alpha chain of IL15Rα can be encoded by a nucleic acid including

(SEQ ID NO: 12)
ATTACATGCCCCCCTCCCATGAGCGTGGAGCACGCCGACATCTGGG
TGAAGAGCTATAGCCTCTACAGCCGGGAGAGGTATATCTGTAACAG
CGGCTTCAAGAGGAAGGCCGGCACCAGCAGCCTCACCGAGTGCGTG
CTGAATAAGGCTACCAACGTGGCTCACTGGACAACACCCTCTTTAA
AGTGCATCCGG.

In some embodiments, a soluble IL-15 can include a sequence that is at least 70% identical, at least 75% identical, at least 80% identical, at least 85% identical, at least 90% identical, at least 95% identical, at least 99% identical, or 100% identical to NWVNVISDLKKIEDLIQSMHIDATLYTESDVHPSCKVTAMKCFLLELQVISLESGD ASIHDTVENLIILANNSLSSNGNVTESGCKECEELEEKNIKEFLQSFVHIVQMFINT S (SEQ ID NO:13). In some embodiments, a soluble IL-15 can be encoded by a nucleic acid including the sequence of

(SEQ ID NO: 14)
AACTGGGTGAACGTCATCAGCGATTTAAAGAAGATCGAAGATTTAA
TTCAGTCCATGCATATCGACGCCACTTTATACACAGAATCCGACGT
GCACCCCTCTTGTAAGGTGACCGCCATGAAATGTTTTTTACTGGAG
CTGCAAGTTATCTCTTTAGAGAGCGGAGACGCTAGCATCCACGACA
CCGTGGAGAATTTAATCATTTTAGCCAATAACTCTTTATCCAGCAA
CGGCAACGTGACAGAGTCCGGCTGCAAGGAGTGCGAAGAGCTGGAG
GAGAAGAACATCAAGGAGTTTCTGCAATCCTTTGTGCACATTGTCC
AGATGTTCATCAATACCTCC.

In some embodiments, a soluble IL-15 can include a D8N amino acid substitution. In some embodiments, the soluble IL-15 with D8N mutant (IL15D8N) can include a sequence that is at least 70% identical, at least 75% identical, at least 80% identical, at least 85% identical, at least 90% identical, at least 92% identical, at least 94% identical, at least 95% identical, at least 96% identical, at least 98% identical, at least 99% identical, or 100% identical to NWVNVISNLKKIEDLIQSMHIDATLYTESDVHPSCKVTAMKCFLLELQVISLESGD ASIHDTVENLIILANNSLSSNGNVTESGCKECEELEEKNIKEFLQSFVHIVQMFINT S (SEQ ID NO:37). In some embodiments, the soluble IL-15 with D8N mutant (IL15D8N) can be encoded by a nucleic acid including the sequence of

(SEQ ID NO: 38)
AACTGGGTGAATGTAATAAGTAATTTGAAAAAAATTGAAGATCTTA
TTCAATCTATGCATATTGATGCTACTTTATATACGGAAAGTGATGT
TCACCCCAGTTGCAAAGTAACAGCAATGAAGTGCTTTCTCTTGGAG
TTACAAGTTATTTCACTTGAGTCCGGAGATGCAAGTATTCATGATA
CAGTAGAAAATCTGATCATCCTAGCAAACAACAGTTTGTCTTCTAA
TGGGAATGTAACAGAATCTGGATGCAAAGAATGTGAGGAACTGGAG
GAAAAAAATATTAAAGAATTTTTGCAGAGTTTTGTACATATTGTCC
AAATGTTCATCAACACTTCT.

### Signal Sequence

In some embodiments, a multi-chain chimeric polypeptide includes a first chimeric polypeptide that includes a signal sequence at its N-terminal end. In some embodiments, a multi-chain chimeric polypeptide includes a second chimeric polypeptide that includes a signal sequence at its N-terminal end. In some embodiments, both the first chimeric polypeptide of a multi-chain chimeric polypeptide and a second chimeric polypeptide of the multi-chain chimeric polypeptide include a signal sequence. As will be understood by those of ordinary skill in the art, a signal sequence is an amino acid sequence that is present at the N-terminus of a number of endogenously produced proteins that directs the protein to the secretory pathway (e.g., the protein is directed to reside in certain intracellular organelles, to reside in the cell membrane, or to be secreted from the cell). Signal sequences are heterogeneous and differ greatly in their primary amino acid sequences. However, signal sequences are typically 16 to 30 amino acids in length and include a hydrophilic, usually positively charged N-terminal region, a central hydrophobic domain, and a C-terminal region that contains the cleavage site for signal peptidase.

In some embodiments, a first chimeric polypeptide of a multi-chain chimeric polypeptide, a second chimeric polypeptide of the multi-chain chimeric polypeptide, or both includes a signal sequence having an amino acid sequence MKWVTFISLLFLFSSAYS (SEQ ID NO:15). In some embodiments, a first chimeric polypeptide of a multi-chain chimeric polypeptide, a second chimeric polypeptide of the multi-chain chimeric polypeptide, or both includes a signal sequence encoded by the nucleic acid sequence

(SEQ ID NO: 16)
ATGAAATGGGTGACCTTTATTTCTTTACTGTTCCTCTTTAGCAGCG
CCTACTCC,
(SEQ ID NO: 17)
ATGAAGTGGGTCACATTTATCTCTTTACTGTTCCTCTTCTCCAGCG
CCTACAGC,
or
(SEQ ID NO: 18)
ATGAAATGGGTGACCTTTATTTCTTTACTGTTCCTCTTTAGCAGCG
CCTACTCC.

In some embodiments, a first chimeric polypeptide of a multi-chain chimeric polypeptide, a second chimeric polypeptide of the multi-chain chimeric polypeptide, or both includes a signal sequence having an amino acid sequence MKCLLYLAFLFLGVNC (SEQ ID NO:19). In some embodiments, a first chimeric polypeptide of a multi-chain chimeric polypeptide, a second chimeric polypeptide of the multi-chain chimeric polypeptide, or both includes a signal sequence having an amino acid sequence MGQIVTMFEALPHIIDEVINIVIIVLIIITSIKAVYNFATCGILALVSFLFLAGRSCG (SEQ ID NO:20). In some embodiments, a first chimeric polypeptide of a multi-chain chimeric polypeptide, a second chimeric polypeptide of the multi-chain chimeric polypeptide, or both includes a signal sequence having an amino acid sequence

(SEQ ID NO: 21)
MPNHQSGSPTGSSDLLLSGKKQRPHLALRRKRRREMRKINRKVRRM
NLAPIKEKTAWQHLQALISEAEEVLKTSQTPQNSLTLFLALLSVLG
PPVTG.

In some embodiments, a first chimeric polypeptide of a multi-chain chimeric polypeptide, a second chimeric polypeptide of the multi-chain chimeric polypeptide, or both includes a signal sequence having an amino acid sequence MDSKGSSQKGSRLLLLLVVSNLLLCQGVVS (SEQ ID NO:22). Those of ordinary skill in the art will be aware of other appropriate signal sequences for use in a first chimeric polypeptide and/or a second chimeric polypeptide of multi-chain chimeric polypeptides described herein.

In some embodiments, a first chimeric polypeptide of a multi-chain chimeric polypeptide, a second chimeric polypeptide of the multi-chain chimeric polypeptide, or both includes a signal sequence that is about 10 to 100 amino acids in length. For example, a signal sequence can be about 10 to 100 amino acids in length, about 15 to 100 amino acids in length, about 20 to 100 amino acids in length, about 25 to 100 amino acids in length, about 30 to 100 amino acids in length, about 35 to 100 amino acids in length, about 40 to 100 amino acids in length, about 45 to 100 amino acids in length, about 50 to 100 amino acids in length, about 55 to 100 amino acids in length, about 60 to 100 amino acids in length, about 65 to 100 amino acids in length, about 70 to 100 amino acids in length, about 75 to 100 amino acids in length, about 80 to 100 amino acids in length, about 85 to 100 amino acids in length, about 90 to 100 amino acids in length, about 95 to 100 amino acids in length, about 10 to 95 amino acids in length, about 10 to 90 amino acids in length, about 10 to 85 amino acids in length, about 10 to 80 amino acids in length, about 10 to 75 amino acids in length, about 10 to 70 amino acids in length, about 10 to 65 amino acids in length, about 10 to 60 amino acids in length, about 10 to 55 amino acids in length, about 10 to 50 amino acids in length, about 10 to 45 amino acids in length, about 10 to 40 amino acids in length, about 10 to 35 amino acids in length, about 10 to 30 amino acids in length, about 10 to 25 amino acids in length, about 10 to 20 amino acids in length, about 10 to 15 amino acids in length, about 20 to 30 amino acids in length, about 30 to 40 amino acids in length, about 40 to 50 amino acids in length, about 50 to 60 amino acids in length, about 60 to 70 amino acids in length, about 70 to 80 amino acids in length, about 80 to 90 amino acids in length, about 90 to 100 amino acids in length, about 20 to 90 amino acids in length, about 30 to 80 amino acids in length, about 40 to 70 amino acids in length, about 50 to 60 amino acids in length, or any range in between. In some embodiments, a signal sequence is about 10, 15, 20, 25, 30, 35, 40, 45, 50, 55, 60, 65, 70, 75, 80, 85, 90, 95, or 100 amino acids in length.

In some embodiments, any of the signal sequences disclosed herein can include one or more additional amino acids (e.g., 1, 2, 3, 5, 6, 7, 8, 9, 10, or more amino acids) at its N-terminus and/or C-terminus, so long as the function of the signal sequence remains intact. For example, a signal sequence having the amino acid sequence MKCLLYLAFLFLGVNC (SEQ ID NO:19) can include one or more additional amino acids at the N-terminus or C-terminus, while still retaining the ability to direct a first chimeric polypeptide of a multi-chain chimeric polypeptide, a second chimeric polypeptide of the multi-chain chimeric polypeptide, or both to the secretory pathway.

In some embodiments, a first chimeric polypeptide of a multi-chain chimeric polypeptide, a second chimeric polypeptide of the multi-chain chimeric polypeptide, or both includes a signal sequence that directs the multi-chain chimeric polypeptide into the extracellular space. Such embodiments are useful in producing multi-chain chimeric polypeptides that are relatively easy to be isolated and/or purified.

### Peptide Tags

In some embodiments, a multi-chain chimeric polypeptide includes a first chimeric polypeptide that includes a peptide tag (e.g., at the N-terminal end or the C-terminal end of the first chimeric polypeptide). In some embodiments, a multi-chain chimeric polypeptide includes a second chimeric polypeptide that includes a peptide tag (e.g., at the N-terminal end or the C-terminal end of the second chimeric polypeptide). In some embodiments, both the first chimeric polypeptide of a multi-chain chimeric polypeptide and a second chimeric polypeptide of the multi-chain chimeric polypeptide include a peptide tag. In some embodiments, a first chimeric polypeptide of a multi-chain chimeric polypeptide, a second chimeric polypeptide of the multi-chain chimeric polypeptide, or both include two or more peptide tags.

Exemplary peptide tags that can be included in a first chimeric polypeptide of a multi-chain chimeric polypeptide, a second chimeric polypeptide of the multi-chain chimeric polypeptide, or both include, without limitation, AviTag, a calmodulin-tag, a polyglutamate tag, an E-tag, a FLAG-tag, an HA-tag, a peptide from hemagglutinin, a his-tag, a myc-tag, NE-tag, S-tag, SBP-tag, Softag 1, Softag 3, Spot-tag, Strep-tag, TC tag, Ty tag, V5 tag, VSV-tag, and Xpress tag. In some embodiments, tissue factor protein is a peptide tag.

Peptide tags that can be included in a first chimeric polypeptide of a multi-chain chimeric polypeptide, a second chimeric polypeptide of the multi-chain chimeric polypeptide, or both can be used in any of a variety of applications related to the multi-chain chimeric polypeptide. For example, a peptide tag can be used in the purification of a multi-chain chimeric polypeptide. As one non-limiting example, a first chimeric polypeptide of a multi-chain chimeric polypeptide (e.g., a recombinantly expressed first chimeric polypeptide), a second chimeric polypeptide of the multi-chain chimeric polypeptide (e.g., a recombinantly expressed second chimeric polypeptide), or both can include a myc tag; the multi-chain chimeric polypeptide that includes the myc-tagged first chimeric polypeptide, the myc-tagged second chimeric polypeptide, or both can be purified using an antibody that recognizes the myc tag(s). One non-limiting example of an antibody that recognizes a myc tag is 9E10, available from the non-commercial Developmental Studies Hybridoma Bank. As another non-limiting example, a first chimeric polypeptide of a multi-chain chimeric polypeptide (e.g., a recombinantly expressed first chimeric polypeptide), a second chimeric polypeptide of the multi-chain chimeric polypeptide (e.g., a recombinantly expressed second chimeric polypeptide), or both can include a histidine tag; the multi-chain chimeric polypeptide that includes the histidine-tagged first chimeric polypeptide, the histidine-tagged second chimeric polypeptide, or both can be purified using a nickel or cobalt chelate. Those of ordinary skill in the art will be aware of other suitable tags and agent that bind those tags for use in purifying multi-chain chimeric polypeptide. In some embodiments, a peptide tag is removed from the first chimeric polypeptide and/or the second chimeric polypeptide of the multi-chain chimeric polypeptide after purification. In some embodiments, a peptide tag is not removed from the first chimeric polypeptide and/or the second chimeric polypeptide of the multi-chain chimeric polypeptide after purification.

Peptide tags that can be included in a first chimeric polypeptide of a multi-chain chimeric polypeptide, a second chimeric polypeptide of the multi-chain chimeric polypeptide, or both can be used, for example, in immunoprecipitation of the multi-chain chimeric polypeptide, imaging of the multi-chain chimeric polypeptide (e.g., via Western blotting, ELISA, flow cytometry, and/or immunocytochemistry), and/or solubilization of the multi-chain chimeric polypeptide.

In some embodiments, a first chimeric polypeptide of a multi-chain chimeric polypeptide, a second chimeric polypeptide of the multi-chain chimeric polypeptide, or both includes a peptide tag that is about 10 to 100 amino acids in length. For example, a peptide tag can be about 10 to 100 amino acids in length, about 15 to 100 amino acids in length, about 20 to 100 amino acids in length, about 25 to 100 amino acids in length, about 30 to 100 amino acids in length, about 35 to 100 amino acids in length, about 40 to 100 amino acids in length, about 45 to 100 amino acids in length, about 50 to 100 amino acids in length, about 55 to 100 amino acids in length, about 60 to 100 amino acids in length, about 65 to 100 amino acids in length, about 70 to 100 amino acids in length, about 75 to 100 amino acids in length, about 80 to 100 amino acids in length, about 85 to 100 amino acids in length, about 90 to 100 amino acids in length, about 95 to 100 amino acids in length, about 10 to 95 amino acids in length, about 10 to 90 amino acids in length, about 10 to 85 amino acids in length, about 10 to 80 amino acids in length, about 10 to 75 amino acids in length, about 10 to 70 amino acids in length, about 10 to 65 amino acids in length, about 10 to 60 amino acids in length, about 10 to 55 amino acids in length, about 10 to 50 amino acids in length, about 10 to 45 amino acids in length, about 10 to 40 amino acids in length, about 10 to 35 amino acids in length, about 10 to 30 amino acids in length, about 10 to 25 amino acids in length, about 10 to 20 amino acids in length, about 10 to 15 amino acids in length, about 20 to 30 amino acids in length, about 30 to 40 amino acids in length, about 40 to 50 amino acids in length, about 50 to 60 amino acids in length, about 60 to 70 amino acids in length, about 70 to 80 amino acids in length, about 80 to 90 amino acids in length, about 90 to 100 amino acids in length, about 20 to 90 amino acids in length, about 30 to 80 amino acids in length, about 40 to 70 amino acids in length, about 50 to 60 amino acids in length, or any range in between. In some embodiments, a peptide tag is about 10, 15, 20, 25, 30, 35, 40, 45, 50, 55, 60, 65, 70, 75, 80, 85, 90, 95, or 100 amino acids in length.

Peptide tags included in a first chimeric polypeptide of a multi-chain chimeric polypeptide, a second chimeric polypeptide of the multi-chain chimeric polypeptide, or both can be of any suitable length. For example, peptide tags can be 5, 6, 7, 8, 9, 10, 11, 12, 13, 14, 15, 16, 17, 18, 19, 20, or more amino acids in length. In embodiments in which a multi-chain chimeric polypeptide includes two or more peptide tags, the two or more peptide tags can be of the same or different lengths. In some embodiments, any of the peptide tags disclosed herein may include one or more additional amino acids (e.g., 1, 2, 3, 5, 6, 7, 8, 9, 10, or more amino acids) at the N-terminus and/or C-terminus, so long as the function of the peptide tag remains intact. For example, a myc tag can include one or more additional amino acids (e.g., at the N-terminus and/or the C-terminus of the peptide tag), while still retaining the ability to be bound by an antibody.

### Exemplary Multi-Chain Chimeric Polypeptides

In some embodiments of any of the multi-chain chimeric polypeptides described herein, the first target-binding domain and the second targeting-binding domain each independently bind specifically to TGF-β. In some examples of these multi-chain chimeric polypeptides, the first target-binding domain and the soluble tissue factor domain directly abut each other in the first chimeric polypeptide. In some examples of these multi-chain chimeric polypeptides, the first chimeric polypeptide further comprises a linker sequence (e.g., any of the exemplary linkers described herein) between the first target-binding domain and the soluble tissue factor domain in the first chimeric polypeptide.

In some embodiments of these multi-chain chimeric polypeptides, the soluble tissue factor domain and the first domain of the pair of affinity domains directly abut each other in the first chimeric polypeptide. In some embodiments of these multi-chain chimeric polypeptides, the first chimeric polypeptide further includes a linker sequence (e.g., any of the exemplary linkers described herein) between the soluble tissue factor domain and the first domain of the pair of affinity domains in the first chimeric polypeptide.

In some embodiments of these multi-chain chimeric polypeptides, the second domain of the pair of affinity domains and the second target-binding domain directly abut each other in the second chimeric polypeptide. In some embodiments of these multi-chain chimeric polypeptides, the second chimeric polypeptide further includes a linker sequence (e.g., any of the exemplary linkers described herein) between the second domain of the pair of affinity domains and the second target-binding domain in the second chimeric polypeptide.

In some embodiments of these multi-chain chimeric polypeptides, the soluble tissue factor domain can be any of the exemplary soluble tissue factor domains described herein. In some embodiments of these multi-chain chimeric polypeptides, the pair of affinity domains can be any of the exemplary pairs of affinity domains described herein.

In some embodiments of these multi-chain chimeric polypeptides, the first target-binding domain and the second target-binding domain each independently bind specifically to TGF-β. In some embodiments of these multi-chain chimeric polypeptides, the first target-binding domain and the second target-binding domain bind specifically to the same epitope. In some embodiments of these multi-chain chimeric polypeptides, the first target-binding domain and the second target-binding domain include the same amino acid sequence.

In some embodiments of these multi-chain chimeric polypeptides, the first target-binding domain and the second target-binding domain is a soluble TGF-β receptor (e.g., a soluble TGFβRII receptor, e.g., a soluble human TGFβRII). In some embodiments of these multi-chain chimeric polypeptides, the soluble human TGFRβRII includes a first sequence of soluble human TGFRβRII and a second sequence of soluble human TGFRβRII. In some embodiments of these multi-chain chimeric polypeptides, the soluble human TGFRβRII includes a linker disposed between the first sequence of soluble human TGFRβRII and the second sequence of soluble human TGFRβRII. In some examples of these multi-chain chimeric polypeptides, the linker includes the sequence GGGGSGGGGSGGGGS (SEQ ID NO:3).

In some embodiments of these multi-chain chimeric polypeptides, the first sequence of soluble human TGFRβRII receptor comprises a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 2)
IPPHVQKSVNNDMIVTDNNGAVKFPQLCKFCDVRFSTCDNQKSCM
SNCSITSICEKPQEVCVAVWRKNDENITLETVCHDPKLPYHDFIL
EDAASPKCIMKEKKKPGETFFMCSCSSDECNDNIIFSEEYNTSNP
D.

In some embodiments of these multi-chain chimeric polypeptides, the second sequence of soluble human TGFRβRII receptor comprises a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 2)
IPPHVQKSVNNDMIVTDNNGAVKFPQLCKFCDVRFSTCDNQKSCM
SNCSITSICEKPQEVCVAVWRKNDENITLETVCHDPKLPYHDFIL
EDAASPKCIMKEKKKPGETFFMCSCSSDECNDNIIFSEEYNTSNP
D.

In some embodiments of these multi-chain chimeric polypeptides, the first sequence of soluble human TGFRβRII receptor is encoded by a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 23)
ATCCCCCCCCATGTGCAAAAGAGCGTGAACAACGATATGATCGTG
ACCGACAACAACGGCGCCGTGAAGTTTCCCCAGCTCTGCAAGTTC
TGCGATGTCAGGTTCAGCACCTGCGATAATCAGAAGTCCTGCATG
TCCAACTGCAGCATCACCTCCATCTGCGAGAAGCCCCAAGAAGTG
TGCGTGGCCGTGTGGCGGAAAAATGACGAGAACATCACCCTGGAG
ACCGTGTGTCACGACCCCAAGCTCCCTTATCACGACTTCATTCTG
GAGGACGCTGCCTCCCCCAAATGCATCATGAAGGAGAAGAAGAAG
CCCGGAGAGACCTTCTTTATGTGTTCCTGTAGCAGCGACGAGTGT
AACGACAACATCATCTTCAGCGAAGAGTACAACACCAGCAACCCT
GAT.

In some embodiments of these multi-chain chimeric polypeptides, the second sequence of soluble human TGFRβRII receptor is encoded by a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical to:

(SEQ ID NO: 24)
ATTCCTCCCCACGTGCAGAAGAGCGTGAATAATGACATGATCGTG
ACCGATAACAATGGCGCCGTGAAATTTCCCCAGCTGTGCAAATTC
TGCGATGTGAGGTTTTCCACCTGCGACAACCAGAAGTCCTGTATG
AGCAACTGCTCCATCACCTCCATCTGTGAGAAGCCTCAGGAGGTG
TGCGTGGCTGTCTGGCGGAAGAATGACGAGAATATCACCCTGGAA
ACCGTCTGCCACGATCCCAAGCTGCCCTACCACGATTTCATCCTG
GAAGACGCCGCCAGCCCTAAGTGCATCATGAAAGAGAAAAAGAAG
CCTGGCGAGACCTTTTTCATGTGCTCCTGCAGCAGCGACGAATGC
AACGACAATATCATCTTTAGCGAGGAATACAATACCAGCAACCCC
GAC.

In some embodiments of these multi-chain chimeric polypeptides, the soluble TGF-β receptor includes a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 4)
IPPHVQKSVNNDMIVTDNNGAVKFPQLCKFCDVRFSTCDNQKSCM
SNCSITSICEKPQEVCVAVWRKNDENITLETVCHDPKLPYHDFIL
EDAASPKCIMKEKKKPGETFFMCSCSSDECNDNIIFSEEYNTSNP
DGGGGSGGGGSGGGGSIPPHVQKSVNNDMIVTDNNGAVKFPQLCK
FCDVRFSTCDNQKSCMSNCSITSICEKPQEVCVAVWRKNDENITL
ETVCHDPKLPYHDFILEDAASPKCIMKEKKKPGETFFMCSCSSDE
CNDNIIFSEEYNTSNPD.

In some embodiments of these multi-chain chimeric polypeptides, the soluble TGF-β receptor is encoded by a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 25)
ATCCCCCCCCATGTGCAAAAGAGCGTGAACAACGATATGATCGTG
ACCGACAACAACGGCGCCGTGAAGTTTCCCCAGCTCTGCAAGTTC
TGCGATGTCAGGTTCAGCACCTGCGATAATCAGAAGTCCTGCATG
TCCAACTGCAGCATCACCTCCATCTGCGAGAAGCCCCAAGAAGTG
TGCGTGGCCGTGTGGCGGAAAAATGACGAGAACATCACCCTGGAG
ACCGTGTGTCACGACCCCAAGCTCCCTTATCACGACTTCATTCTG
GAGGACGCTGCCTCCCCCAAATGCATCATGAAGGAGAAGAAGAAG
CCCGGAGAGACCTTCTTTATGTGTTCCTGTAGCAGCGACGAGTGT
AACGACAACATCATCTTCAGCGAAGAGTACAACACCAGCAACCCT
GATGGAGGTGGCGGATCCGGAGGTGGAGGTTCTGGTGGAGGTGGG
AGTATTCCTCCCCACGTGCAGAAGAGCGTGAATAATGACATGATC
GTGACCGATAACAATGGCGCCGTGAAATTTCCCCAGCTGTGCAAA
TTCTGCGATGTGAGGTTTTCCACCTGCGACAACCAGAAGTCCTGT
ATGAGCAACTGCTCCATCACCTCCATCTGTGAGAAGCCTCAGGAG
GTGTGCGTGGCTGTCTGGCGGAAGAATGACGAGAATATCACCCTG
GAAACCGTCTGCCACGATCCCAAGCTGCCCTACCACGATTTCATC
CTGGAAGACGCCGCCAGCCCTAAGTGCATCATGAAAGAGAAAAAG
AAGCCTGGCGAGACCTTTTTCATGTGCTCCTGCAGCAGCGACGAA
TGCAACGACAATATCATCTTTAGCGAGGAATACAATACCAGCAAC
CCCGAC.

In some embodiments, the first chimeric polypeptide can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 6)
IPPHVQKSVNNDMIVTDNNGAVKFPQLCKFCDVRFSTCDNQKSCM
SNCSITSICEKPQEVCVAVWRKNDENITLETVCHDPKLPYHDFIL
EDAASPKCIMKEKKKPGETFFMCSCSSDECNDNIIFSEEYNTSNP
DGGGGSGGGGSGGGGSIPPHVQKSVNNDMIVTDNNGAVKFPQLCK
FCDVRFSTCDNQKSCMSNCSITSICEKPQEVCVAVWRKNDENITL
ETVCHDPKLPYHDFILEDAASPKCIMKEKKKPGETFFMCSCSSDE
CNDNIIFSEEYNTSNPDSGTTNTVAAYNLTWKSTNFKTILEWEPK
PVNQVYTVQISTKSGDWKSKCFYTTDTECDLTDEIVKDVKQTYLA
RVFSYPAGNVESTGSAGEPLYENSPEFTPYLETNLGQPTIQSFEQ
VGTKVNVTVEDERTLVRRNNTFLSLRDVFGKDLIYTLYYWKSSSS
GKKTAKTNTNEFLIDVDKGENYCFSVQAVIPSRTVNRKSTDSPVE
CMGQEKGEFRENWVNVISDLKKIEDLIQSMHIDATLYTESDVHPS
CKVTAMKCFLLELQVISLESGDASIHDTVENLIILANNSLSSNGN
VTESGCKECEELEEKNIKEFLQSFVHIVQMFINTS.

In some embodiments, a first chimeric polypeptide is encoded by a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 26)
ATCCCCCCCCATGTGCAAAAGAGCGTGAACAACGATATGATCGTG
ACCGACAACAACGGCGCCGTGAAGTTTCCCCAGCTCTGCAAGTTC
TGCGATGTCAGGTTCAGCACCTGCGATAATCAGAAGTCCTGCATG
TCCAACTGCAGCATCACCTCCATCTGCGAGAAGCCCCAAGAAGTG
TGCGTGGCCGTGTGGCGGAAAAATGACGAGAACATCACCCTGGAG
ACCGTGTGTCACGACCCCAAGCTCCCTTATCACGACTTCATTCTG
GAGGACGCTGCCTCCCCCAAATGCATCATGAAGGAGAAGAAGAAG
CCCGGAGAGACCTTCTTTATGTGTTCCTGTAGCAGCGACGAGTGT
AACGACAACATCATCTTCAGCGAAGAGTACAACACCAGCAACCCT
GATGGAGGTGGCGGATCCGGAGGTGGAGGTTCTGGTGGAGGTGGG
AGTATTCCTCCCCACGTGCAGAAGAGCGTGAATAATGACATGATC
GTGACCGATAACAATGGCGCCGTGAAATTTCCCCAGCTGTGCAAA
TTCTGCGATGTGAGGTTTTCCACCTGCGACAACCAGAAGTCCTGT
ATGAGCAACTGCTCCATCACCTCCATCTGTGAGAAGCCTCAGGAG
GTGTGCGTGGCTGTCTGGCGGAAGAATGACGAGAATATCACCCTG
GAAACCGTCTGCCACGATCCCAAGCTGCCCTACCACGATTTCATC
CTGGAAGACGCCGCCAGCCCTAAGTGCATCATGAAAGAGAAAAAG
AAGCCTGGCGAGACCTTTTTCATGTGCTCCTGCAGCAGCGACGAA
TGCAACGACAATATCATCTTTAGCGAGGAATACAATACCAGCAAC
CCCGACAGCGGCACAACCAACACAGTCGCTGCCTATAACCTCACT
TGGAAGAGCACCAACTTCAAAACCATCCTCGAATGGGAACCCAAA
CCCGTTAACCAAGTTTACACCGTGCAGATCAGCACCAAGTCCGGC
GACTGGAAGTCCAAATGTTTCTATACCACCGACACCGAGTGCGAT
CTCACCGATGAGATCGTGAAAGATGTGAAACAGACCTACCTCGCC
CGGGTGTTTAGCTACCCCGCCGGCAATGTGGAGAGCACTGGTTCC
GCTGGCGAGCCTTTATACGAGAACAGCCCCGAATTTACCCCTTAC
CTCGAGACCAATTTAGGACAGCCCACCATCCAAAGCTTTGAGCAA
GTTGGCACAAAGGTGAATGTGACAGTGGAGGACGAGCGGACTTTA
GTGCGGCGGAACAACACCTTTCTCAGCCTCCGGGATGTGTTCGGC
AAAGATTTAATCTACACACTGTATTACTGGAAGTCCTCTTCCTCC
GGCAAGAAGACAGCTAAAACCAACACAAACGAGTTTTTAATCGAC
GTGGATAAAGGCGAAAACTACTGTTTCAGCGTGCAAGCTGTGATC
CCCTCCCGGACCGTGAATAGGAAAAGCACCGATAGCCCCGTTGAG
TGCATGGGCCAAGAAAAGGGCGAGTTCCGGGAGAACTGGGTGAAC
GTCATCAGCGATTTAAAGAAGATCGAAGATTTAATTCAGTCCATG
CATATCGACGCCACTTTATACACAGAATCCGACGTGCACCCCTCT
TGTAAGGTGACCGCCATGAAATGTTTTTTACTGGAGCTGCAAGTT
ATCTCTTTAGAGAGCGGAGACGCTAGCATCCACGACACCGTGGAG
AATTTAATCATTTTAGCCAATAACTCTTTATCCAGCAACGGCAAC
GTGACAGAGTCCGGCTGCAAGGAGTGCGAAGAGCTGGAGGAGAAG
AACATCAAGGAGTTTCTGCAATCCTTTGTGCACATTGTCCAGATG
TTCATCAATACCTCC.

In some embodiments, a first chimeric polypeptide can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 7)
MKWVTFISLLFLFSSAYSIPPHVQKSVNNDMIVTDNNGAVKFPQL
CKFCDVRFSTCDNQKSCMSNCSITSICEKPQEVCVAVWRKNDENI
TLETVCHDPKLPYHDFILEDAASPKCIMKEKKKPGETFFMCSCSS
DECNDNIIFSEEYNTSNPDGGGGSGGGGSGGGGSIPPHVQKSVNN
DMIVTDNNGAVKFPQLCKFCDVRFSTCDNQKSCMSNCSITSICEK
PQEVCVAVWRKNDENITLETVCHDPKLPYHDFILEDAASPKCIMK
EKKKPGETFFMCSCSSDECNDNIIFSEEYNTSNPDSGTTNTVAAY
NLTWKSTNFKTILEWEPKPVNQVYTVQISTKSGDWKSKCFYTTDT
ECDLTDEIVKDVKQTYLARVFSYPAGNVESTGSAGEPLYENSPEF
TPYLETNLGQPTIQSFEQVGTKVNVTVEDERTLVRRNNTFLSLRD
VFGKDLIYTLYYWKSSSSGKKTAKTNTNEFLIDVDKGENYCFSVQ
AVIPSRTVNRKSTDSPVECMGQEKGEFRENWVNVISDLKKIEDLI
QSMHIDATLYTESDVHPSCKVTAMKCFLLELQVISLESGDASIHD
TVENLIILANNSLSSNGNVTESGCKECEELEEKNIKEFLQSFVHI
VQMFINTS.

In some embodiments, a first chimeric polypeptide is encoded by a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical to:

(SEQ ID NO: 27)
ATGAAGTGGGTGACCTTCATCAGCCTGCTGTTCCTGTTCTCCAGC
GCCTACTCCATCCCCCCCCATGTGCAAAAGAGCGTGAACAACGAT
ATGATCGTGACCGACAACAACGGCGCCGTGAAGTTTCCCCAGCTC
TGCAAGTTCTGCGATGTCAGGTTCAGCACCTGCGATAATCAGAAG
TCCTGCATGTCCAACTGCAGCATCACCTCCATCTGCGAGAAGCCC
CAAGAAGTGTGCGTGGCCGTGTGGCGGAAAAATGACGAGAACATC
ACCCTGGAGACCGTGTGTCACGACCCCAAGCTCCCTTATCACGAC
TTCATTCTGGAGGACGCTGCCTCCCCCAAATGCATCATGAAGGAG
AAGAAGAAGCCCGGAGAGACCTTCTTTATGTGTTCCTGTAGCAGC
GACGAGTGTAACGACAACATCATCTTCAGCGAAGAGTACAACACC
AGCAACCCTGATGGAGGTGGCGGATCCGGAGGTGGAGGTTCTGGT
GGAGGTGGGAGTATTCCTCCCCACGTGCAGAAGAGCGTGAATAAT
GACATGATCGTGACCGATAACAATGGCGCCGTGAAATTTCCCCAG
CTGTGCAAATTCTGCGATGTGAGGTTTTCCACCTGCGACAACCAG
AAGTCCTGTATGAGCAACTGCTCCATCACCTCCATCTGTGAGAAG
CCTCAGGAGGTGTGCGTGGCTGTCTGGCGGAAGAATGACGAGAAT
ATCACCCTGGAAACCGTCTGCCACGATCCCAAGCTGCCCTACCAC
GATTTCATCCTGGAAGACGCCGCCAGCCCTAAGTGCATCATGAAA
GAGAAAAAGAAGCCTGGCGAGACCTTTTTCATGTGCTCCTGCAGC
AGCGACGAATGCAACGACAATATCATCTTTAGCGAGGAATACAAT
ACCAGCAACCCCGACAGCGGCACAACCAACACAGTCGCTGCCTAT
AACCTCACTTGGAAGAGCACCAACTTCAAAACCATCCTCGAATGG
GAACCCAAACCCGTTAACCAAGTTTACACCGTGCAGATCAGCACC
AAGTCCGGCGACTGGAAGTCCAAATGTTTCTATACCACCGACACC
GAGTGCGATCTCACCGATGAGATCGTGAAAGATGTGAAACAGACC
TACCTCGCCCGGGTGTTTAGCTACCCCGCCGGCAATGTGGAGAGC
ACTGGTTCCGCTGGCGAGCCTTTATACGAGAACAGCCCCGAATTT
ACCCCTTACCTCGAGACCAATTTAGGACAGCCCACCATCCAAAGC
TTTGAGCAAGTTGGCACAAAGGTGAATGTGACAGTGGAGGACGAG
CGGACTTTAGTGCGGCGGAACAACACCTTTCTCAGCCTCCGGGAT
GTGTTCGGCAAAGATTTAATCTACACACTGTATTACTGGAAGTCC
TCTTCCTCCGGCAAGAAGACAGCTAAAACCAACACAAACGAGTTT
TTAATCGACGTGGATAAAGGCGAAAACTACTGTTTCAGCGTGCAA
GCTGTGATCCCCTCCCGGACCGTGAATAGGAAAAGCACCGATAGC
CCCGTTGAGTGCATGGGCCAAGAAAAGGGCGAGTTCCGGGAGAAC
TGGGTGAACGTCATCAGCGATTTAAAGAAGATCGAAGATTTAATT
CAGTCCATGCATATCGACGCCACTTTATACACAGAATCCGACGTG
CACCCCTCTTGTAAGGTGACCGCCATGAAATGTTTTTTACTGGAG
CTGCAAGTTATCTCTTTAGAGAGCGGAGACGCTAGCATCCACGAC
ACCGTGGAGAATTTAATCATTTTAGCCAATAACTCTTTATCCAGC
AACGGCAACGTGACAGAGTCCGGCTGCAAGGAGTGCGAAGAGCTG
GAGGAGAAGAACATCAAGGAGTTTCTGCAATCCTTTGTGCACATT
GTCCAGATGTTCATCAATACCTCC.

In some embodiments, the second chimeric polypeptide can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 5)
IPPHVQKSVNNDMIVTDNNGAVKFPQLCKFCDVRFSTCDNQKSCM
SNCSITSICEKPQEVCVAVWRKNDENITLETVCHDPKLPYHDFIL
EDAASPKCIMKEKKKPGETFFMCSCSSDECNDNIIFSEEYNTSNP
DGGGGSGGGGSGGGGSIPPHVQKSVNNDMIVTDNNGAVKFPQLCK
FCDVRFSTCDNQKSCMSNCSITSICEKPQEVCVAVWRKNDENITL
ETVCHDPKLPYHDFILEDAASPKCIMKEKKKPGETFFMCSCSSDE
CNDNIIFSEEYNTSNPDITCPPPMSVEHADIWVKSYSLYSRERYI
CNSGFKRKAGTSSLTECVLNKATNVAHWTTPSLKCIR.

In some embodiments, a second chimeric polypeptide is encoded by a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 28)
ATCCCCCCCCATGTGCAAAAGAGCGTGAACAACGATATGATCGTG
ACCGACAACAACGGCGCCGTGAAGTTTCCCCAGCTCTGCAAGTTC
TGCGATGTCAGGTTCAGCACCTGCGATAATCAGAAGTCCTGCATG
TCCAACTGCAGCATCACCTCCATCTGCGAGAAGCCCCAAGAAGTG
TGCGTGGCCGTGTGGCGGAAAAATGACGAGAACATCACCCTGGAG
ACCGTGTGTCACGACCCCAAGCTCCCTTATCACGACTTCATTCTG
GAGGACGCTGCCTCCCCCAAATGCATCATGAAGGAGAAGAAGAAG
CCCGGAGAGACCTTCTTTATGTGTTCCTGTAGCAGCGACGAGTGT
AACGACAACATCATCTTCAGCGAAGAGTACAACACCAGCAACCCT
GATGGAGGTGGCGGATCCGGAGGTGGAGGTTCTGGTGGAGGTGGG
AGTATTCCTCCCCACGTGCAGAAGAGCGTGAATAATGACATGATC
GTGACCGATAACAATGGCGCCGTGAAATTTCCCCAGCTGTGCAAA
TTCTGCGATGTGAGGTTTTCCACCTGCGACAACCAGAAGTCCTGT
ATGAGCAACTGCTCCATCACCTCCATCTGTGAGAAGCCTCAGGAG
GTGTGCGTGGCTGTCTGGCGGAAGAATGACGAGAATATCACCCTG
GAAACCGTCTGCCACGATCCCAAGCTGCCCTACCACGATTTCATC
CTGGAAGACGCCGCCAGCCCTAAGTGCATCATGAAAGAGAAAAAG
AAGCCTGGCGAGACCTTTTTCATGTGCTCCTGCAGCAGCGACGAA
TGCAACGACAATATCATCTTTAGCGAGGAATACAATACCAGCAAC
CCCGACATTACATGCCCCCCTCCCATGAGCGTGGAGCACGCCGAC
ATCTGGGTGAAGAGCTATAGCCTCTACAGCCGGGAGAGGTATATC
TGTAACAGCGGCTTCAAGAGGAAGGCCGGCACCAGCAGCCTCACC
GAGTGCGTGCTGAATAAGGCTACCAACGTGGCTCACTGGACAACA
CCCTCTTTAAAGTGCATCCGG.

In some embodiments, a second chimeric polypeptide can include a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 8)
MKWVTFISLLFLFSSAYSIPPHVQKSVNNDMIVTDNNGAVKFPQL
CKFCDVRFSTCDNQKSCMSNCSITSICEKPQEVCVAVWRKNDENI
TLETVCHDPKLPYHDFILEDAASPKCIMKEKKKPGETFFMCSCSS
DECNDNIIFSEEYNTSNPDGGGGSGGGGSGGGGSIPPHVQKSVNN
DMIVTDNNGAVKFPQLCKFCDVRFSTCDNQKSCMSNCSITSICEK
PQEVCVAVWRKNDENITLETVCHDPKLPYHDFILEDAASPKCIMK
EKKKPGETFFMCSCSSDECNDNIIFSEEYNTSNPDITCPPPMSVE
HADIWVKSYSLYSRERYICNSGFKRKAGTSSLTECVLNKATNVAH
WTTPSLKCIR.

In some embodiments, a second chimeric polypeptide is encoded by a sequence that is at least 80% identical (e.g., at least 82% identical, at least 84% identical, at least 86% identical, at least 88% identical, at least 90% identical, at least 92% identical, at least 94% identical, at least 96% identical, at least 98% identical, at least 99% identical, or 100% identical) to:

(SEQ ID NO: 29)
ATGAAGTGGGTGACCTTCATCAGCCTGCTGTTCCTGTTCTCCAGC
GCCTACTCCATCCCCCCCCATGTGCAAAAGAGCGTGAACAACGAT
ATGATCGTGACCGACAACAACGGCGCCGTGAAGTTTCCCCAGCTC
TGCAAGTTCTGCGATGTCAGGTTCAGCACCTGCGATAATCAGAAG
TCCTGCATGTCCAACTGCAGCATCACCTCCATCTGCGAGAAGCCC
CAAGAAGTGTGCGTGGCCGTGTGGCGGAAAAATGACGAGAACATC
ACCCTGGAGACCGTGTGTCACGACCCCAAGCTCCCTTATCACGAC
TTCATTCTGGAGGACGCTGCCTCCCCCAAATGCATCATGAAGGAG
AAGAAGAAGCCCGGAGAGACCTTCTTTATGTGTTCCTGTAGCAGC
GACGAGTGTAACGACAACATCATCTTCAGCGAAGAGTACAACACC
AGCAACCCTGATGGAGGTGGCGGATCCGGAGGTGGAGGTTCTGGT
GGAGGTGGGAGTATTCCTCCCCACGTGCAGAAGAGCGTGAATAAT
GACATGATCGTGACCGATAACAATGGCGCCGTGAAATTTCCCCAG
CTGTGCAAATTCTGCGATGTGAGGTTTTCCACCTGCGACAACCAG
AAGTCCTGTATGAGCAACTGCTCCATCACCTCCATCTGTGAGAAG
CCTCAGGAGGTGTGCGTGGCTGTCTGGCGGAAGAATGACGAGAAT
ATCACCCTGGAAACCGTCTGCCACGATCCCAAGCTGCCCTACCAC
GATTTCATCCTGGAAGACGCCGCCAGCCCTAAGTGCATCATGAAA
GAGAAAAAGAAGCCTGGCGAGACCTTTTTCATGTGCTCCTGCAGC
AGCGACGAATGCAACGACAATATCATCTTTAGCGAGGAATACAAT
ACCAGCAACCCCGACATTACATGCCCCCCTCCCATGAGCGTGGAG
CACGCCGACATCTGGGTGAAGAGCTATAGCCTCTACAGCCGGGAG
AGGTATATCTGTAACAGCGGCTTCAAGAGGAAGGCCGGCACCAGC
AGCCTCACCGAGTGCGTGCTGAATAAGGCTACCAACGTGGCTCAC
TGGACAACACCCTCTTTAAAGTGCATCCGG.

In some embodiments, the first chimeric polypeptide can include a sequence that is at least 80% identical (e.g., at least 82% identical, at least 84% identical, at least 85% identical, at least 86% identical, at least 88% identical, at least 90% identical, at least 92% identical, at least 94% identical, at least 95% identical, at least 96% identical, at least 98% identical, at least 99% identical, or 100% identical to:

(SEQ ID NO: 30)
IPPHVQKSVNNDMIVTDNNGAVKFPQLCKFCDVRFSTCDNQKSCM
SNCSITSICEKPQEVCVAVWRKNDENITLETVCHDPKLPYHDFIL
EDAASPKCIMKEKKKPGETFFMCSCSSDECNDNIIFSEEYNTSNP
DGGGGSGGGGSGGGGSIPPHVQKSVNNDMIVTDNNGAVKFPQLCK
FCDVRFSTCDNQKSCMSNCSITSICEKPQEVCVAVWRKNDENITL
ETVCHDPKLPYHDFILEDAASPKCIMKEKKKPGETFFMCSCSSDE
CNDNIIFSEEYNTSNPDITCPPPMSVEHADIWVKSYSLYSRERYI
CNSGFKRKAGTSSLTECVLNKATNVAHWTTPSLKCIR.

In some embodiments, a first chimeric polypeptide is encoded by a sequence that is at least 80% identical (e.g., at least 82% identical, at least 84% identical, at least 85% identical, at least 86% identical, at least 88% identical, at least 90% identical, at least 92% identical, at least 94% identical, at least 95% identical, at least 96% identical, at least 98% identical, at least 99% identical, or 100% identical) to:

(SEQ ID NO: 31)
ATCCCCCCCCATGTGCAAAAGAGCGTGAACAACGATATGATCGTG
ACCGACAACAACGGCGCCGTGAAGTTTCCCCAGCTCTGCAAGTTC
TGCGATGTCAGGTTCAGCACCTGCGATAATCAGAAGTCCTGCATG
TCCAACTGCAGCATCACCTCCATCTGCGAGAAGCCCCAAGAAGTG
TGCGTGGCCGTGTGGCGGAAAAATGACGAGAACATCACCCTGGAG
ACCGTGTGTCACGACCCCAAGCTCCCTTATCACGACTTCATTCTG
GAGGACGCTGCCTCCCCCAAATGCATCATGAAGGAGAAGAAGAAG
CCCGGAGAGACCTTCTTTATGTGTTCCTGTAGCAGCGACGAGTGT
AACGACAACATCATCTTCAGCGAAGAGTACAACACCAGCAACCCT
GATGGAGGTGGCGGATCCGGAGGTGGAGGTTCTGGTGGAGGTGGG
AGTATTCCTCCCCACGTGCAGAAGAGCGTGAATAATGACATGATC
GTGACCGATAACAATGGCGCCGTGAAATTTCCCCAGCTGTGCAAA
TTCTGCGATGTGAGGTTTTCCACCTGCGACAACCAGAAGTCCTGT
ATGAGCAACTGCTCCATCACCTCCATCTGTGAGAAGCCTCAGGAG
GTGTGCGTGGCTGTCTGGCGGAAGAATGACGAGAATATCACCCTG
GAAACCGTCTGCCACGATCCCAAGCTGCCCTACCACGATTTCATC
CTGGAAGACGCCGCCAGCCCTAAGTGCATCATGAAAGAGAAAAAG
AAGCCTGGCGAGACCTTTTTCATGTGCTCCTGCAGCAGCGACGAA
TGCAACGACAATATCATCTTTAGCGAGGAATACAATACCAGCAAC
CCCGACATTACATGCCCCCCTCCCATGAGCGTGGAGCACGCCGAC
ATCTGGGTGAAGAGCTATAGCCTCTACAGCCGGGAGAGGTATATC
TGTAACAGCGGCTTCAAGAGGAAGGCCGGCACCAGCAGCCTCACC
GAGTGCGTGCTGAATAAGGCTACCAACGTGGCTCACTGGACAACA
CCCTCTTTAAAGTGCATCCGG.

In some embodiments, the first chimeric polypeptide can include a sequence that is at least 80% identical (e.g., at least 82% identical, at least 84% identical, at least 85% identical, at least 86% identical, at least 88% identical, at least 90% identical, at least 92% identical, at least 94% identical, at least 95% identical, at least 96% identical, at least 98% identical, at least 99% identical, or 100% identical) to:

(SEQ ID NO: 8)
MKWVTFISLLFLFSSAYSIPPHVQKSVNNDMIVTDNNGAVKFPQLC
KFCDVRFSTCDNQKSCMSNCSITSICEKPQEVCVAVWRKNDENITL
ETVCHDPKLPYHDFILEDAASPKCIMKEKKKPGETFFMCSCSSDEC
NDNIIFSEEYNTSNPDGGGGSGGGGSGGGGSIPPHVQKSVNNDMIV
TDNNGAVKFPQLCKFCDVRFSTCDNQKSCMSNCSITSICEKPQEVC
VAVWRKNDENITLETVCHDPKLPYHDFILEDAASPKCIMKEKKKPG
ETFFMCSCSSDECNDNIIFSEEYNTSNPDITCPPPMSVEHADIWVK
SYSLYSRERYICNSGFKRKAGTSSLTECVLNKATNVAHWTTPSLKC
IR.

In some embodiments, a first chimeric polypeptide is encoded by a sequence that is at least 80% identical (e.g., at least 82% identical, at least 84% identical, at least 85% identical, at least 86% identical, at least 88% identical, at least 90% identical, at least 92% identical, at least 94% identical, at least 95% identical, at least 96% identical, at least 98% identical, at least 99% identical, or 100% identical) to:

(SEQ ID NO: 32)
ATGAAGTGGGTGACCTTCATCAGCCTGCTGTTCCTGTTCTCCAGCG
CCTACTCCATCCCCCCCCATGTGCAAAAGAGCGTGAACAACGATAT
GATCGTGACCGACAACAACGGCGCCGTGAAGTTTCCCCAGCTCTGC
AAGTTCTGCGATGTCAGGTTCAGCACCTGCGATAATCAGAAGTCCT
GCATGTCCAACTGCAGCATCACCTCCATCTGCGAGAAGCCCCAAGA
AGTGTGCGTGGCCGTGTGGCGGAAAAATGACGAGAACATCACCCTG
GAGACCGTGTGTCACGACCCCAAGCTCCCTTATCACGACTTCATTC
TGGAGGACGCTGCCTCCCCCAAATGCATCATGAAGGAGAAGAAGAA
GCCCGGAGAGACCTTCTTTATGTGTTCCTGTAGCAGCGACGAGTGT
AACGACAACATCATCTTCAGCGAAGAGTACAACACCAGCAACCCTG
ATGGAGGTGGCGGATCCGGAGGTGGAGGTTCTGGTGGAGGTGGGAG
TATTCCTCCCCACGTGCAGAAGAGCGTGAATAATGACATGATCGTG
ACCGATAACAATGGCGCCGTGAAATTTCCCCAGCTGTGCAAATTCT
GCGATGTGAGGTTTTCCACCTGCGACAACCAGAAGTCCTGTATGAG
CAACTGCTCCATCACCTCCATCTGTGAGAAGCCTCAGGAGGTGTGC
GTGGCTGTCTGGCGGAAGAATGACGAGAATATCACCCTGGAAACCG
TCTGCCACGATCCCAAGCTGCCCTACCACGATTTCATCCTGGAAGA
CGCCGCCAGCCCTAAGTGCATCATGAAAGAGAAAAAGAAGCCTGGC
GAGACCTTTTTCATGTGCTCCTGCAGCAGCGACGAATGCAACGACA
ATATCATCTTTAGCGAGGAATACAATACCAGCAACCCCGACATTAC
ATGCCCCCCTCCCATGAGCGTGGAGCACGCCGACATCTGGGTGAAG
AGCTATAGCCTCTACAGCCGGGAGAGGTATATCTGTAACAGCGGCT
TCAAGAGGAAGGCCGGCACCAGCAGCCTCACCGAGTGCGTGCTGAA
TAAGGCTACCAACGTGGCTCACTGGACAACACCCTCTTTAAAGTGC
ATCCGG.

In some embodiments, a second chimeric polypeptide can include a sequence that is at least 80% identical (e.g., at least 82% identical, at least 84% identical, at least 85% identical, at least 86% identical, at least 88% identical, at least 90% identical, at least 92% identical, at least 94% identical, at least 95% identical, at least 96% identical, at least 98% identical, at least 99% identical, or 100% identical) to:

(SEQ ID NO: 33)
IPPHVQKSVNNDMIVTDNNGAVKFPQLCKFCDVRFSTCDNQKSCMS
NCSITSICEKPQEVCVAVWRKNDENITLETVCHDPKLPYHDFILED
AASPKCIMKEKKKPGETFFMCSCSSDECNDNIIFSEEYNTSNPDGG
GGSGGGGSGGGGSIPPHVQKSVNNDMIVTDNNGAVKFPQLCKFCDV
RFSTCDNQKSCMSNCSITSICEKPQEVCVAVWRKNDENITLETVCH
DPKLPYHDFILEDAASPKCIMKEKKKPGETFFMCSCSSDECNDNII
FSEEYNTSNPDSGTTNTVAAYNLTWKSTNFKTILEWEPKPVNQVYT
VQISTKSGDWKSKCFYTTDTECDLTDEIVKDVKQTYLARVFSYPAG
NVESTGSAGEPLYENSPEFTPYLETNLGQPTIQSFEQVGTKVNVTV
EDERTLVRRNNTFLSLRDVFGKDLIYTLYYWKSSSSGKKTAKTNTN
EFLIDVDKGENYCFSVQAVIPSRTVNRKSTDSPVECMGQEKGEFRE
NWVNVISNLKKIEDLIQSMHIDATLYTESDVHPSCKVTAMKCFLLE
LQVISLESGDASIHDTVENLIILANNSLSSNGNVTESGCKECEELE
EKNIKEFLQSFVHIVQMFINTS.

In some embodiments, a second chimeric polypeptide is encoded by a sequence that is at least 80% identical (e.g., at least 82% identical, at least 84% identical, at least 85% identical, at least 86% identical, at least 88% identical, at least 90% identical, at least 92% identical, at least 94% identical, at least 95% identical, at least 96% identical, at least 98% identical, at least 99% identical, or 100% identical) to:

(SEQ ID NO: 34)
ATCCCACCGCACGTTCAGAAGTCGGTGAATAACGACATGATAGTCA
CTGACAACAACGGTGCAGTCAAGTTTCCACAACTGTGTAAATTTTG
TGATGTGAGATTTTCCACCTGTGACAACCAGAAATCCTGCATGAGC
AACTGCAGCATCACCTCCATCTGTGAGAAGCCACAGGAAGTCTGTG
TGGCTGTATGGAGAAAGAATGACGAGAACATAACACTAGAGACAGT
TTGCCATGACCCCAAGCTCCCCTACCATGACTTTATTCTGGAAGAT
GCTGCTTCTCCAAAGTGCATTATGAAGGAAAAAAAAAAGCCTGGTG
AGACTTTCTTCATGTGTTCCTGTAGCTCTGATGAGTGCAATGACAA
CATCATCTTCTCAGAAGAATATAACACCAGCAATCCTGACGGAGGT
GGCGGATCCGGAGGTGGAGGTTCTGGTGGAGGTGGGAGTATTCCTC
CCCACGTGCAGAAGAGCGTGAATAATGACATGATCGTGACCGATAA
CAATGGCGCCGTGAAATTTCCCCAGCTGTGCAAATTCTGCGATGTG
AGGTTTTCCACCTGCGACAACCAGAAGTCCTGTATGAGCAACTGCT
CCATCACCTCCATCTGTGAGAAGCCTCAGGAGGTGTGCGTGGCTGT
CTGGCGGAAGAATGACGAGAATATCACCCTGGAAACCGTCTGCCAC
GATCCCAAGCTGCCCTACCACGATTTCATCCTGGAAGACGCCGCCA
GCCCTAAGTGCATCATGAAAGAGAAAAAGAAGCCTGGCGAGACCTT
TTTCATGTGCTCCTGCAGCAGCGACGAATGCAACGACAATATCATC
TTTAGCGAGGAATACAATACCAGCAACCCCGACTCAGGCACTACAA
ATACTGTGGCAGCATATAATTTAACTTGGAAATCAACTAATTTCAA
GACAATTTTGGAGTGGGAACCCAAACCCGTCAATCAAGTCTACACT
GTTCAAATAAGCACTAAGTCAGGAGATTGGAAAAGCAAATGCTTTT
ACACAACAGACACAGAGTGTGACCTCACCGACGAGATTGTGAAGGA
TGTGAAGCAGACGTACTTGGCACGGGTCTTCTCCTACCCGGCAGGG
AATGTGGAGAGCACCGGTTCTGCTGGGGAGCCTCTGTATGAGAACT
CCCCAGAGTTCACACCTTACCTGGAGACAAACCTCGGACAGCCAAC
AATTCAGAGTTTTGAACAGGTGGGAACAAAAGTGAATGTGACCGTA
GAAGATGAACGGACTTTAGTCAGAAGGAACAACACTTTCCTAAGCC
TCCGGGATGTTTTTGGCAAGGACTTAATTTATACACTTTATTATTG
GAAATCTTCAAGTTCAGGAAAGAAAACAGCCAAAACAAACACTAAT
GAGTTTTTGATTGATGTGGATAAAGGAGAAAACTACTGTTTCAGTG
TTCAAGCAGTGATTCCCTCCCGAACAGTTAACCGGAAGAGTACAGA
CAGCCCGGTAGAGTGTATGGGCCAGGAGAAAGGGGAATTCAGAGAA
AACTGGGTGAATGTAATAAGTAATTTGAAAAAAATTGAAGATCTTA
TTCAATCTATGCATATTGATGCTACTTTATATACGGAAAGTGATGT
TCACCCCAGTTGCAAAGTAACAGCAATGAAGTGCTTTCTCTTGGAG
TTACAAGTTATTTCACTTGAGTCCGGAGATGCAAGTATTCATGATA
CAGTAGAAAATCTGATCATCCTAGCAAACAACAGTTTGTCTTCTAA
TGGGAATGTAACAGAATCTGGATGCAAAGAATGTGAGGAACTGGAG
GAAAAAAATATTAAAGAATTTTTGCAGAGTTTTGTACATATTGTCC
AAATGTTCATCAACACTTCT.

In some embodiments, a second chimeric polypeptide can include a sequence that is at least 80% identical (e.g., at least 82% identical, at least 84% identical, at least 85% identical, at least 86% identical, at least 88% identical, at least 90% identical, at least 92% identical, at least 94% identical, at least 95% identical, at least 96% identical, at least 98% identical, at least 99% identical, or 100% identical to:

(SEQ ID NO: 35)
MGVKVLFALICIAVAEAIPPHVQKSVNNDMIVTDNNGAVKFPQLCK
FCDVRFSTCDNQKSCMSNCSITSICEKPQEVCVAVWRKNDENITLE
TVCHDPKLPYHDFILEDAASPKCIMKEKKKPGETFFMCSCSSDECN
DNIIFSEEYNTSNPDGGGGSGGGGSGGGGSIPPHVQKSVNNDMIVT
DNNGAVKFPQLCKFCDVRFSTCDNQKSCMSNCSITSICEKPQEVCV
AVWRKNDENITLETVCHDPKLPYHDFILEDAASPKCIMKEKKKPGE
TFFMCSCSSDECNDNIIFSEEYNTSNPDSGTTNTVAAYNLTWKSTN
FKTILEWEPKPVNQVYTVQISTKSGDWKSKCFYTTDTECDLTDEIV
KDVKQTYLARVFSYPAGNVESTGSAGEPLYENSPEFTPYLETNLGQ
PTIQSFEQVGTKVNVTVEDERTLVRRNNTFLSLRDVFGKDLIYTLY
YWKSSSSGKKTAKTNTNEFLIDVDKGENYCFSVQAVIPSRTVNRKS
TDSPVECMGQEKGEFRENWVNVISNLKKIEDLIQSMHIDATLYTES
DVHPSCKVTAMKCFLLELQVISLESGDASIHDTVENLIILANNSLS
SNGNVTESGCKECEELEEKNIKEFLQSFVHIVQMFINTS.

In some embodiments, a second chimeric polypeptide is encoded by a sequence that is at least 80% identical (e.g., at least 82% identical, at least 84% identical, at least 85% identical, at least 86% identical, at least 88% identical, at least 90% identical, at least 92% identical, at least 94% identical, at least 95% identical, at least 96% identical, at least 98% identical at least 99% identical or 100% identical to:

(SEQ ID NO: 36)
ATGGGAGTGAAAGTTCTTTTTGCCCTTATTTGTATTGCTGTGGCCG
AGGCCATCCCACCGCACGTTCAGAAGTCGGTGAATAACGACATGAT
AGTCACTGACAACAACGGTGCAGTCAAGTTTCCACAACTGTGTAAA
TTTTGTGATGTGAGATTTTCCACCTGTGACAACCAGAAATCCTGCA
TGAGCAACTGCAGCATCACCTCCATCTGTGAGAAGCCACAGGAAGT
CTGTGTGGCTGTATGGAGAAAGAATGACGAGAACATAACACTAGAG
ACAGTTTGCCATGACCCCAAGCTCCCCTACCATGACTTTATTCTGG
AAGATGCTGCTTCTCCAAAGTGCATTATGAAGGAAAAAAAAAAGCC
TGGTGAGACTTTCTTCATGTGTTCCTGTAGCTCTGATGAGTGCAAT
GACAACATCATCTTCTCAGAAGAATATAACACCAGCAATCCTGACG
GAGGTGGCGGATCCGGAGGTGGAGGTTCTGGTGGAGGTGGGAGTAT
TCCTCCCCACGTGCAGAAGAGCGTGAATAATGACATGATCGTGACC
GATAACAATGGCGCCGTGAAATTTCCCCAGCTGTGCAAATTCTGCG
ATGTGAGGTTTTCCACCTGCGACAACCAGAAGTCCTGTATGAGCAA
CTGCTCCATCACCTCCATCTGTGAGAAGCCTCAGGAGGTGTGCGTG
GCTGTCTGGCGGAAGAATGACGAGAATATCACCCTGGAAACCGTCT
GCCACGATCCCAAGCTGCCCTACCACGATTTCATCCTGGAAGACGC
CGCCAGCCCTAAGTGCATCATGAAAGAGAAAAAGAAGCCTGGCGAG
ACCTTTTTCATGTGCTCCTGCAGCAGCGACGAATGCAACGACAATA
TCATCTTTAGCGAGGAATACAATACCAGCAACCCCGACTCAGGCAC
TACAAATACTGTGGCAGCATATAATTTAACTTGGAAATCAACTAAT
TTCAAGACAATTTTGGAGTGGGAACCCAAACCCGTCAATCAAGTCT
ACACTGTTCAAATAAGCACTAAGTCAGGAGATTGGAAAAGCAAATG
CTTTTACACAACAGACACAGAGTGTGACCTCACCGACGAGATTGTG
AAGGATGTGAAGCAGACGTACTTGGCACGGGTCTTCTCCTACCCGG
CAGGGAATGTGGAGAGCACCGGTTCTGCTGGGGAGCCTCTGTATGA
GAACTCCCCAGAGTTCACACCTTACCTGGAGACAAACCTCGGACAG
CCAACAATTCAGAGTTTTGAACAGGTGGGAACAAAAGTGAATGTGA
CCGTAGAAGATGAACGGACTTTAGTCAGAAGGAACAACACTTTCCT
AAGCCTCCGGGATGTTTTTGGCAAGGACTTAATTTATACACTTTAT
TATTGGAAATCTTCAAGTTCAGGAAAGAAAACAGCCAAAACAAACA
CTAATGAGTTTTTGATTGATGTGGATAAAGGAGAAAACTACTGTTT
CAGTGTTCAAGCAGTGATTCCCTCCCGAACAGTTAACCGGAAGAGT
ACAGACAGCCCGGTAGAGTGTATGGGCCAGGAGAAAGGGGAATTCA
GAGAAAACTGGGTGAATGTAATAAGTAATTTGAAAAAAATTGAAGA
TCTTATTCAATCTATGCATATTGATGCTACTTTATATACGGAAAGT
GATGTTCACCCCAGTTGCAAAGTAACAGCAATGAAGTGCTTTCTCT
TGGAGTTACAAGTTATTTCACTTGAGTCCGGAGATGCAAGTATTCA
TGATACAGTAGAAAATCTGATCATCCTAGCAAACAACAGTTTGTCT
TCTAATGGGAATGTAACAGAATCTGGATGCAAAGAATGTGAGGAAC
TGGAGGAAAAAAATATTAAAGAATTTTTGCAGAGTTTTGTACATAT
TGTCCAAATGTTCATCAACACTTCT.

### Compositions/Kits

Also provided herein are compositions (e.g., pharmaceutical compositions) that include at least one of any multi-chain chimeric polypeptides, any of the cells, or any of the nucleic acids described herein. In some embodiments, the compositions include at least one of any of the multi-chain chimeric polypeptides described herein. In some embodiments, the compositions include any of the immune cells (e.g., any of the immune cells described herein, e.g., any of the immune cells produced using any of the methods described herein).

In some embodiments, the pharmaceutical compositions are formulated for different routes of administration (e.g., intravenous, subcutaneous). In some embodiments, the pharmaceutical compositions can include a pharmaceutically acceptable carrier (e.g., phosphate buffered saline).

Single or multiple administrations of pharmaceutical compositions can be given to a subject in need thereof depending on for example: the dosage and frequency as required and tolerated by the subject. The formulation should provide a sufficient quantity of active agent to effectively treat, prevent or ameliorate conditions, diseases or symptoms.

Also provided herein are kits that include any of the multi-chain chimeric polypeptides, compositions, nucleic acids, or cells (e.g., immune cells) described herein. In some embodiments, the kits can include instructions for performing any of the methods described herein. In some embodiments, the kits can include at least one dose of any of the pharmaceutical compositions described herein.

### Nucleic Acids/Vectors

Also provided herein are nucleic acids that encode any of the multi-chain chimeric polypeptides described herein. In some embodiments, a first nucleic acid can encode the first chimeric polypeptide and a second nucleic acid can encode the second chimeric polypeptide. In some embodiments, a single nucleic acid can encode both the first chimeric polypeptide and the second chimeric polypeptide.

Also provided herein are vectors that include any of the nucleic acids encoding any of the multi-chain chimeric polypeptides described herein. In some embodiments, a first vector can include a nucleic acid encoding the first chimeric polypeptide and a second vector can include a nucleic acid encoding the second chimeric polypeptide. In some embodiments, a single vector can include a first nucleic acid encoding the first chimeric polypeptide and a second nucleic acid encoding the second chimeric polypeptide.

Any of the vectors described herein can be an expression vector. For example, an expression vector can include a promoter sequence operably linked to the sequence encoding the first chimeric polypeptide and the second chimeric polypeptide.

Non-limiting examples of vectors include plasmids, transposons, cosmids, and viral vectors (e.g., any adenoviral vectors (e.g., pSV or pCMV vectors), adeno-associated virus (AAV) vectors, lentivirus vectors, and retroviral vectors), and any Gateway® vectors. A vector can, e.g., include sufficient cis-acting elements for expression; other elements for expression can be supplied by the host mammalian cell or in an in vitro expression system. Skilled practitioners will be capable of selecting suitable vectors and mammalian cells for making any of the multi-chain chimeric polypeptides described herein.

### Cells

Also provided herein are cells (e.g., any of the exemplary cells described herein or known in the art) comprising any of the nucleic acids described herein that encode any of the multi-chain chimeric polypeptides described herein (e.g., encoding both the first and second chimeric polypeptides). Also provided herein are cells (e.g., any of the exemplary cells described herein or known in the art) comprising any of the nucleic acids described herein that encode any of the first chimeric polypeptides described herein. Also provided are cells (e.g., any of the exemplary cells described herein or known in the art) comprising any of the nucleic acids described herein that encode any of the second chimeric polypeptides described herein.

Also provided herein are cells (e.g., any of the exemplary cells described herein or known in the art) that include any of the vectors described herein that encode any of the multi-chain chimeric polypeptides described herein (e.g., encoding both the first and second chimeric polypeptides). Also provided herein are cells (e.g., any of the exemplary cells described herein or known in the art) that include any of the vectors described herein that encode any of the first chimeric polypeptides described herein. Also provided herein are cells (e.g., any of the exemplary cells described herein or known in the art) that include any of the vectors described herein that encode any of the second chimeric polypeptides described herein).

In some embodiments of any of the methods described herein, the cell can be a eukaryotic cell. As used herein, the term “eukaryotic cell” refers to a cell having a distinct, membrane-bound nucleus. Such cells may include, for example, mammalian (e.g., rodent, non-human primate, or human), insect, fungal, or plant cells. In some embodiments, the eukaryotic cell is a yeast cell, such as Saccharomyces cerevisiae. In some embodiments, the eukaryotic cell is a higher eukaryote, such as mammalian, avian, plant, or insect cells. Non-limiting examples of mammalian cells include Chinese hamster ovary cells and human embryonic kidney cells (e.g., HEK293 cells).

Methods of introducing nucleic acids and expression vectors into a cell (e.g., a eukaryotic cell) are known in the art. Non-limiting examples of methods that can be used to introduce a nucleic acid into a cell include lipofection, transfection, electroporation, microinjection, calcium phosphate transfection, dendrimer-based transfection, cationic polymer transfection, cell squeezing, sonoporation, optical transfection, impalefection, hydrodynamic delivery, magnetofection, viral transduction (e.g., adenoviral and lentiviral transduction), and nanoparticle transfection.

### Methods of Producing Multi-Chain Chimeric Polypeptides

Also provided herein are methods of producing any of the multi-chain chimeric polypeptides described herein that include culturing any of the cells described herein in a culture medium under conditions sufficient to result in the production of the multi-chain chimeric polypeptide; and recovering the multi-chain chimeric polypeptide from the cell and/or the culture medium.

Also provided herein are method of producing any of the multi-chain chimeric polypeptides described herein that include: culturing any of cells described herein in a first culture medium under conditions sufficient to result in the production of the first chimeric polypeptide; recovering the first chimeric polypeptide from the cell and/or the first culture medium; culturing any of the cells described herein in a second culture medium under conditions sufficient to result in the production of the second chimeric polypeptide; recovering the second chimeric polypeptide from the cell and/or the second culture medium; and combining (e.g., mixing) the recovered first chimeric polypeptide and the recovered second chimeric polypeptide to form the multi-chain chimeric polypeptide (e.g., any of the multi-chain chimeric polypeptides described herein).

The recovery of the multi-chain chimeric polypeptide, the first chimeric polypeptide, or the second chimeric polypeptide from a cell (e.g., a eukaryotic cell) can be performed using techniques well-known in the art (e.g., ammonium sulfate precipitation, polyethylene glycol precipitation, ion-exchange chromatography (anion or cation), chromatography based on hydrophobic interaction, metal-affinity chromatography, ligand-affinity chromatography, and size exclusion chromatography).

Methods of culturing cells are well known in the art. Cells can be maintained in vitro under conditions that favor proliferation, differentiation and growth. Briefly, cells can be cultured by contacting a cell (e.g., any cell) with a cell culture medium that includes the necessary growth factors and supplements to support cell viability and growth.

Also provided herein are multi-chain chimeric polypeptides (e.g., any of the multi-chain chimeric polypeptides described herein), first chimeric polypeptides (e.g., any of the first chimeric polypeptides), or second chimeric polypeptides (e.g., any of the second chimeric polypeptides described herein) produced by any of the methods described herein.

### Methods of Treating a Solid Advanced Tumor or a Solid Advanced Cancer

Provided herein are methods of treating an unresectable, advanced, recurrent, and/or metastatic solid cancer or an unresectable, advanced, recurrent, and/or metastatic solid tumor in a subject that include administering to the subject two or more doses of about 0.2 mg/kg to about 1.3 mg/kg (e.g., about 0.2 mg/kg to about 1.25 mg/mg, about 0.2 mg/kg to about 1.2 mg/mg, about 0.2 mg/kg to about 1.15 mg/kg, about 0.2 mg/kg to about 1.1 mg/kg, about 0.2 mg/kg to about 1.05 mg/kg, about 0.2 mg/kg to about 1.0 mg/kg, about 0.2 mg/kg to about 0.95 mg/kg, about 0.2 mg/kg to about 0.9 mg/kg, about 0.2 mg/kg to about 0.85 mg/kg, about 0.2 mg/kg to about 0.8 mg/kg, about 0.2 mg/kg to about 0.75 mg/kg, about 0.2 mg/kg to about 0.7 mg/kg, about 0.2 mg/kg to about 0.65 mg/kg, about 0.2 mg/kg to about 0.6 mg/kg, about 0.2 mg/kg to about 0.55 mg/kg, about 0.2 mg/kg to about 0.5 mg/kg, about 0.2 mg/kg to about 0.45 mg/kg, about 0.2 mg/kg to about 0.4 mg/kg, about 0.2 mg/kg to about 0.35 mg/kg, about 0.2 mg/kg to about 0.3 mg/kg, about 0.25 mg/kg to about 1.3 mg/kg, about 0.25 mg/kg to about 1.25 mg/mg, about 0.25 mg/kg to about 1.2 mg/mg, about 0.25 mg/kg to about 1.15 mg/kg, about 0.25 mg/kg to about 1.1 mg/kg, about 0.25 mg/kg to about 1.05 mg/kg, about 0.25 mg/kg to about 1.0 mg/kg, about 0.25 mg/kg to about 0.95 mg/kg, about 0.25 mg/kg to about 0.9 mg/kg, about 0.25 mg/kg to about 0.85 mg/kg, about 0.25 mg/kg to about 0.8 mg/kg, about 0.25 mg/kg to about 0.75 mg/kg, about 0.25 mg/kg to about 0.7 mg/kg, about 0.25 mg/kg to about 0.65 mg/kg, about 0.25 mg/kg to about 0.6 mg/kg, about 0.25 mg/kg to about 0.55 mg/kg, about 0.25 mg/kg to about 0.5 mg/kg, about 0.25 mg/kg to about 0.45 mg/kg, about 0.25 mg/kg to about 0.4 mg/kg, about 0.25 mg/kg to about 0.35 mg/kg, about 0.3 mg/kg to about 1.3 mg/kg, about 0.3 mg/kg to about 1.25 mg/mg, about 0.3 mg/kg to about 1.2 mg/mg, about 0.3 mg/kg to about 1.15 mg/kg, about 0.3 mg/kg to about 1.1 mg/kg, about 0.3 mg/kg to about 1.05 mg/kg, about 0.3 mg/kg to about 1.0 mg/kg, about 0.3 mg/kg to about 0.95 mg/kg, about 0.3 mg/kg to about 0.9 mg/kg, about 0.3 mg/kg to about 0.85 mg/kg, about 0.3 mg/kg to about 0.8 mg/kg, about 0.3 mg/kg to about 0.75 mg/kg, about 0.3 mg/kg to about 0.7 mg/kg, about 0.3 mg/kg to about 0.65 mg/kg, about 0.3 mg/kg to about 0.6 mg/kg, about 0.3 mg/kg to about 0.55 mg/kg, about 0.3 mg/kg to about 0.5 mg/kg, about 0.3 mg/kg to about 0.45 mg/kg, about 0.3 mg/kg to about 0.4 mg/kg, about 0.35 mg/kg to about 1.3 mg/kg, about 0.35 mg/kg to about 1.25 mg/mg, about 0.35 mg/kg to about 1.2 mg/mg, about 0.35 mg/kg to about 1.15 mg/kg, about 0.35 mg/kg to about 1.1 mg/kg, about 0.35 mg/kg to about 1.05 mg/kg, about 0.35 mg/kg to about 1.0 mg/kg, about 0.35 mg/kg to about 0.95 mg/kg, about 0.35 mg/kg to about 0.9 mg/kg, about 0.35 mg/kg to about 0.85 mg/kg, about 0.35 mg/kg to about 0.8 mg/kg, about 0.35 mg/kg to about 0.75 mg/kg, about 0.35 mg/kg to about 0.7 mg/kg, about 0.35 mg/kg to about 0.65 mg/kg, about 0.35 mg/kg to about 0.6 mg/kg, about 0.35 mg/kg to about 0.55 mg/kg, about 0.35 mg/kg to about 0.5 mg/kg, about 0.35 mg/kg to about 0.45 mg/kg, about 0.4 mg/kg to about 1.3 mg/kg, about 0.4 mg/kg to about 1.25 mg/mg, about 0.4 mg/kg to about 1.2 mg/mg, about 0.4 mg/kg to about 1.15 mg/kg, about 0.4 mg/kg to about 1.1 mg/kg, about 0.4 mg/kg to about 1.05 mg/kg, about 0.4 mg/kg to about 1.0 mg/kg, about 0.4 mg/kg to about 0.95 mg/kg, about 0.4 mg/kg to about 0.9 mg/kg, about 0.4 mg/kg to about 0.85 mg/kg, about 0.4 mg/kg to about 0.8 mg/kg, about 0.4 mg/kg to about 0.75 mg/kg, about 0.4 mg/kg to about 0.7 mg/kg, about 0.4 mg/kg to about 0.65 mg/kg, about 0.4 mg/kg to about 0.6 mg/kg, about 0.4 mg/kg to about 0.55 mg/kg, about 0.4 mg/kg to about 0.5 mg/kg, about 0.45 mg/kg to about 1.3 mg/kg, about 0.45 mg/kg to about 1.25 mg/mg, about 0.45 mg/kg to about 1.2 mg/mg, about 0.45 mg/kg to about 1.15 mg/kg, about 0.45 mg/kg to about 1.1 mg/kg, about 0.45 mg/kg to about 1.05 mg/kg, about 0.45 mg/kg to about 1.0 mg/kg, about 0.45 mg/kg to about 0.95 mg/kg, about 0.45 mg/kg to about 0.9 mg/kg, about 0.45 mg/kg to about 0.85 mg/kg, about 0.45 mg/kg to about 0.8 mg/kg, about 0.45 mg/kg to about 0.75 mg/kg, about 0.45 mg/kg to about 0.7 mg/kg, about 0.45 mg/kg to about 0.65 mg/kg, about 0.45 mg/kg to about 0.6 mg/kg, about 0.45 mg/kg to about 0.55 mg/kg, about 0.5 mg/kg to about 1.3 mg/kg, about 0.5 mg/kg to about 1.25 mg/mg, about 0.5 mg/kg to about 1.2 mg/mg, about 0.5 mg/kg to about 1.15 mg/kg, about 0.5 mg/kg to about 1.1 mg/kg, about 0.5 mg/kg to about 1.05 mg/kg, about 0.5 mg/kg to about 1.0 mg/kg, about 0.5 mg/kg to about 0.95 mg/kg, about 0.5 mg/kg to about 0.9 mg/kg, about 0.5 mg/kg to about 0.85 mg/kg, about 0.5 mg/kg to about 0.8 mg/kg, about 0.5 mg/kg to about 0.75 mg/kg, about 0.5 mg/kg to about 0.7 mg/kg, about 0.5 mg/kg to about 0.65 mg/kg, about 0.5 mg/kg to about 0.6 mg/kg, about 0.55 mg/kg to about 1.3 mg/kg, about 0.55 mg/kg to about 1.25 mg/mg, about 0.55 mg/kg to about 1.2 mg/mg, about 0.55 mg/kg to about 1.15 mg/kg, about 0.55 mg/kg to about 1.1 mg/kg, about 0.55 mg/kg to about 1.05 mg/kg, about 0.55 mg/kg to about 1.0 mg/kg, about 0.55 mg/kg to about 0.95 mg/kg, about 0.55 mg/kg to about 0.9 mg/kg, about 0.55 mg/kg to about 0.85 mg/kg, about 0.55 mg/kg to about 0.8 mg/kg, about 0.55 mg/kg to about 0.75 mg/kg, about 0.55 mg/kg to about 0.7 mg/kg, about 0.55 mg/kg to about 0.65 mg/kg, about 0.6 mg/kg to about 1.3 mg/kg, about 0.6 mg/kg to about 1.25 mg/mg, about 0.6 mg/kg to about 1.2 mg/mg, about 0.6 mg/kg to about 1.15 mg/kg, about 0.6 mg/kg to about 1.1 mg/kg, about 0.6 mg/kg to about 1.05 mg/kg, about 0.6 mg/kg to about 1.0 mg/kg, about 0.6 mg/kg to about 0.95 mg/kg, about 0.6 mg/kg to about 0.9 mg/kg, about 0.6 mg/kg to about 0.85 mg/kg, about 0.6 mg/kg to about 0.8 mg/kg, about 0.6 mg/kg to about 0.75 mg/kg, about 0.6 mg/kg to about 0.7 mg/kg, about 0.65 mg/kg to about 1.3 mg/kg, about 0.65 mg/kg to about 1.25 mg/mg, about 0.65 mg/kg to about 1.2 mg/mg, about 0.65 mg/kg to about 1.15 mg/kg, about 0.65 mg/kg to about 1.1 mg/kg, about 0.65 mg/kg to about 1.05 mg/kg, about 0.65 mg/kg to about 1.0 mg/kg, about 0.65 mg/kg to about 0.95 mg/kg, about 0.65 mg/kg to about 0.9 mg/kg, about 0.65 mg/kg to about 0.85 mg/kg, about 0.65 mg/kg to about 0.8 mg/kg, about 0.65 mg/kg to about 0.75 mg/kg, about 0.7 mg/kg to about 1.3 mg/kg, about 0.7 mg/kg to about 1.25 mg/mg, about 0.7 mg/kg to about 1.2 mg/mg, about 0.7 mg/kg to about 1.15 mg/kg, about 0.7 mg/kg to about 1.1 mg/kg, about 0.7 mg/kg to about 1.05 mg/kg, about 0.7 mg/kg to about 1.0 mg/kg, about 0.7 mg/kg to about 0.95 mg/kg, about 0.7 mg/kg to about 0.9 mg/kg, about 0.7 mg/kg to about 0.85 mg/kg, about 0.7 mg/kg to about 0.8 mg/kg, about 0.75 mg/kg to about 1.3 mg/kg, about 0.75 mg/kg to about 1.25 mg/mg, about 0.75 mg/kg to about 1.2 mg/mg, about 0.75 mg/kg to about 1.15 mg/kg, about 0.75 mg/kg to about 1.1 mg/kg, about 0.75 mg/kg to about 1.05 mg/kg, about 0.75 mg/kg to about 1.0 mg/kg, about 0.75 mg/kg to about 0.95 mg/kg, about 0.75 mg/kg to about 0.9 mg/kg, about 0.75 mg/kg to about 0.85 mg/kg, about 0.8 mg/kg to about 1.3 mg/kg, about 0.8 mg/kg to about 1.25 mg/mg, about 0.8 mg/kg to about 1.2 mg/mg, about 0.8 mg/kg to about 1.15 mg/kg, about 0.8 mg/kg to about 1.1 mg/kg, about 0.8 mg/kg to about 1.05 mg/kg, about 0.8 mg/kg to about 1.0 mg/kg, about 0.8 mg/kg to about 0.95 mg/kg, about 0.8 mg/kg to about 0.9 mg/kg, about 0.85 mg/kg to about 1.3 mg/kg, about 0.85 mg/kg to about 1.25 mg/mg, about 0.85 mg/kg to about 1.2 mg/mg, about 0.85 mg/kg to about 1.15 mg/kg, about 0.85 mg/kg to about 1.1 mg/kg, about 0.85 mg/kg to about 1.05 mg/kg, about 0.85 mg/kg to about 1.0 mg/kg, about 0.85 mg/kg to about 0.95 mg/kg, about 0.9 mg/kg to about 1.3 mg/kg, about 0.9 mg/kg to about 1.25 mg/mg, about 0.9 mg/kg to about 1.2 mg/mg, about 0.9 mg/kg to about 1.15 mg/kg, about 0.9 mg/kg to about 1.1 mg/kg, about 0.9 mg/kg to about 1.05 mg/kg, about 0.9 mg/kg to about 1.0 mg/kg, about 0.95 mg/kg to about 1.3 mg/kg, about 0.95 mg/kg to about 1.25 mg/mg, about 0.95 mg/kg to about 1.2 mg/mg, about 0.95 mg/kg to about 1.15 mg/kg, about 0.95 mg/kg to about 1.1 mg/kg, about 0.95 mg/kg to about 1.05 mg/kg, about 1.0 mg/kg to about 1.3 mg/kg, about 1.0 mg/kg to about 1.25 mg/mg, about 1.0 mg/kg to about 1.2 mg/mg, about 1.0 mg/kg to about 1.15 mg/kg, about 1.0 mg/kg to about 1.1 mg/kg, about 1.05 mg/kg to about 1.3 mg/kg, about 1.05 mg/kg to about 1.25 mg/mg, about 1.05 mg/kg to about 1.2 mg/mg, about 1.05 mg/kg to about 1.15 mg/kg, about 1.1 mg/kg to about 1.3 mg/kg, about 1.1 mg/kg to about 1.25 mg/mg, about 1.1 mg/kg to about 1.2 mg/mg, about 1.15 mg/kg to about 1.3 mg/kg, about 1.15 mg/kg to about 1.25 mg/mg, or about 1.2 mg/kg to about 1.3 mg/kg) of a multi-chain chimeric polypeptide, wherein the time between administration of any two consecutive doses of the two or more doses is about 15 days to about 27 days (e.g., about 15 days to about 26 days, about 15 days to about 25 days, about 15 days to about 24 days, about 15 days to about 23 days, about 15 days to about 22 days, about 15 days to about 21 days, about 15 days to about 20 days, about 15 days to about 19 days, about 15 days to about 18 days, about 15 days to about 17 days, about 16 days to about 27 days, about 16 days to about 26 days, about 16 days to about 25 days, about 16 days to about 24 days, about 16 days to about 23 days, about 16 days to about 22 days, about 16 days to about 21 days, about 16 days to about 20 days, about 16 days to about 19 days, about 16 days to about 18 days, about 17 days to about 27 days, about 17 days to about 26 days, about 17 days to about 25 days, about 17 days to about 24 days, about 17 days to about 23 days, about 17 days to about 22 days, about 17 days to about 21 days, about 17 days to about 20 days, about 17 days to about 19 days, about 18 days to about 27 days, about 18 days to about 26 days, about 18 days to about 25 days, about 18 days to about 24 days, about 18 days to about 23 days, about 18 days to about 22 days, about 18 days to about 21 days, about 18 days to about 20 days, about 19 days to about 27 days, about 19 days to about 26 days, about 19 days to about 25 days, about 19 days to about 24 days, about 19 days to about 23 days, about 19 days to about 22 days, about 19 days to about 21 days, about 20 days to about 27 days, about 20 days to about 26 days, about 20 days to about 25 days, about 20 days to about 24 days, about 20 days to about 23 days, about 20 days to about 22 days, about 21 days to about 27 days, about 21 days to about 26 days, about 21 days to about 25 days, about 21 days to about 24 days, about 21 days to about 23 days, about 22 days to about 27 days, about 22 days to about 26 days, about 22 days to about 25 days, about 22 days to about 24 days, about 23 days to about 27 days, about 23 days to about 26 days, about 23 days to about 25 days, about 24 days to about 27 days, about 24 days to about 26 days, or about 25 days to about 27 days), where the multi-chain chimeric polypeptide comprises: (a) a first chimeric polypeptide comprising: (i) a first target-binding domain; (ii) a soluble tissue factor domain; and (iii) a first domain of a pair of affinity domains; and (b) a second chimeric polypeptide comprising: (i) a second domain of a pair of affinity domains; and (ii) a second target-binding domain, where: the first chimeric polypeptide and the second chimeric polypeptide associate through the binding of the first domain and the second domain of the pair of affinity domains; and the first target-binding domain binds specifically to a ligand of TGF-β receptor II (TGF-βRII) and the second target-binding domain binds specifically to a ligand of TGF-βRII. Any of the exemplary multi-chain chimeric polypeptides described herein can be used in these methods.

Also provided herein are methods of treating an unresectable, advanced, recurrent, and/or metastatic solid cancer or an unresectable, advanced, recurrent, and/or metastatic solid tumor in a subject that include administering to the subject two or more doses of about 20 mg to about 150 mg (e.g., about 20 mg to about 145 mg, about 20 mg to about 140 mg, about 20 mg to about 135 mg, about 20 mg to about 130 mg, about 20 mg to about 125 mg, about 20 mg to about 120 mg, about 20 mg to about 115 mg, about 20 mg to about 110 mg, about 20 mg to about 105 mg, about 20 mg to about 100 mg, about 20 mg to about 95 mg, about 20 mg to about 90 mg, about 20 mg to about 85 mg, about 20 mg to about 80 mg, about 20 mg to about 75 mg, about 20 mg to about 70 mg, about 20 mg to about 65 mg, about 20 mg to about 60 mg, about 20 mg to about 55 mg, about 20 mg to about 50 mg, about 20 mg to about 45 mg, about 20 mg to about 40 mg, about 20 mg to about 35 mg, about 20 mg to about 30 mg, about 25 mg to about 150 mg, about 25 mg to about 145 mg, about 25 mg to about 140 mg, about 25 mg to about 135 mg, about 25 mg to about 130 mg, about 25 mg to about 125 mg, about 25 mg to about 120 mg, about 25 mg to about 115 mg, about 25 mg to about 110 mg, about 25 mg to about 105 mg, about 25 mg to about 100 mg, about 25 mg to about 95 mg, about 25 mg to about 90 mg, about 25 mg to about 85 mg, about 25 mg to about 80 mg, about 25 mg to about 75 mg, about 25 mg to about 70 mg, about 25 mg to about 65 mg, about 25 mg to about 60 mg, about 25 mg to about 55 mg, about 25 mg to about 50 mg, about 25 mg to about 45 mg, about 25 mg to about 40 mg, about 25 mg to about 35 mg, about 30 mg to about 150 mg, about 30 mg to about 145 mg, about 30 mg to about 140 mg, about 30 mg to about 135 mg, about 30 mg to about 130 mg, about 30 mg to about 125 mg, about 30 mg to about 120 mg, about 30 mg to about 115 mg, about 30 mg to about 110 mg, about 30 mg to about 105 mg, about 30 mg to about 100 mg, about 30 mg to about 95 mg, about 30 mg to about 90 mg, about 30 mg to about 85 mg, about 30 mg to about 80 mg, about 30 mg to about 75 mg, about 30 mg to about 70 mg, about 30 mg to about 65 mg, about 30 mg to about 60 mg, about 30 mg to about 55 mg, about 30 mg to about 50 mg, about 30 mg to about 45 mg, about 30 mg to about 40 mg, about 35 mg to about 150 mg, about 35 mg to about 145 mg, about 35 mg to about 140 mg, about 35 mg to about 135 mg, about 35 mg to about 130 mg, about 35 mg to about 125 mg, about 35 mg to about 120 mg, about 35 mg to about 115 mg, about 35 mg to about 110 mg, about 35 mg to about 105 mg, about 35 mg to about 100 mg, about 35 mg to about 95 mg, about 35 mg to about 90 mg, about 35 mg to about 85 mg, about 35 mg to about 80 mg, about 35 mg to about 75 mg, about 35 mg to about 70 mg, about 35 mg to about 65 mg, about 35 mg to about 60 mg, about 35 mg to about 55 mg, about 35 mg to about 50 mg, about 35 mg to about 45 mg, about 40 mg to about 150 mg, about 40 mg to about 145 mg, about 40 mg to about 140 mg, about 40 mg to about 135 mg, about 40 mg to about 130 mg, about 40 mg to about 125 mg, about 40 mg to about 120 mg, about 40 mg to about 115 mg, about 40 mg to about 110 mg, about 40 mg to about 105 mg, about 40 mg to about 100 mg, about 40 mg to about 95 mg, about 40 mg to about 90 mg, about 40 mg to about 85 mg, about 40 mg to about 80 mg, about 40 mg to about 75 mg, about 40 mg to about 70 mg, about 40 mg to about 65 mg, about 40 mg to about 60 mg, about 40 mg to about 55 mg, about 40 mg to about 50 mg, about 45 mg to about 150 mg, about 45 mg to about 145 mg, about 45 mg to about 140 mg, about 45 mg to about 135 mg, about 45 mg to about 130 mg, about 45 mg to about 125 mg, about 45 mg to about 120 mg, about 45 mg to about 115 mg, about 45 mg to about 110 mg, about 45 mg to about 105 mg, about 45 mg to about 100 mg, about 45 mg to about 95 mg, about 45 mg to about 90 mg, about 45 mg to about 85 mg, about 45 mg to about 80 mg, about 45 mg to about 75 mg, about 45 mg to about 70 mg, about 45 mg to about 65 mg, about 45 mg to about 60 mg, about 45 mg to about 55 mg, about 50 mg to about 150 mg, about 50 mg to about 145 mg, about 50 mg to about 140 mg, about 50 mg to about 135 mg, about 50 mg to about 130 mg, about 50 mg to about 125 mg, about 50 mg to about 120 mg, about 50 mg to about 115 mg, about 50 mg to about 110 mg, about 50 mg to about 105 mg, about 50 mg to about 100 mg, about 50 mg to about 95 mg, about 50 mg to about 90 mg, about 50 mg to about 85 mg, about 50 mg to about 80 mg, about 50 mg to about 75 mg, about 50 mg to about 70 mg, about 50 mg to about 65 mg, about 50 mg to about 60 mg, about 55 mg to about 150 mg, about 55 mg to about 145 mg, about 55 mg to about 140 mg, about 55 mg to about 135 mg, about 55 mg to about 130 mg, about 55 mg to about 125 mg, about 55 mg to about 120 mg, about 55 mg to about 115 mg, about 55 mg to about 110 mg, about 55 mg to about 105 mg, about 55 mg to about 100 mg, about 55 mg to about 95 mg, about 55 mg to about 90 mg, about 55 mg to about 85 mg, about 55 mg to about 80 mg, about 55 mg to about 75 mg, about 55 mg to about 70 mg, about 55 mg to about 65 mg, about 60 mg to about 150 mg, about 60 mg to about 145 mg, about 60 mg to about 140 mg, about 60 mg to about 135 mg, about 60 mg to about 130 mg, about 60 mg to about 125 mg, about 60 mg to about 120 mg, about 60 mg to about 115 mg, about 60 mg to about 110 mg, about 60 mg to about 105 mg, about 60 mg to about 100 mg, about 60 mg to about 95 mg, about 60 mg to about 90 mg, about 60 mg to about 85 mg, about 60 mg to about 80 mg, about 60 mg to about 75 mg, about 60 mg to about 70 mg, about 65 mg to about 150 mg, about 65 mg to about 145 mg, about 65 mg to about 140 mg, about 65 mg to about 135 mg, about 65 mg to about 130 mg, about 65 mg to about 125 mg, about 65 mg to about 120 mg, about 65 mg to about 115 mg, about 65 mg to about 110 mg, about 65 mg to about 105 mg, about 65 mg to about 100 mg, about 65 mg to about 95 mg, about 65 mg to about 90 mg, about 65 mg to about 85 mg, about 65 mg to about 80 mg, about 65 mg to about 75 mg, about 70 mg to about 150 mg, about 70 mg to about 145 mg, about 70 mg to about 140 mg, about 70 mg to about 135 mg, about 70 mg to about 130 mg, about 70 mg to about 125 mg, about 70 mg to about 120 mg, about 70 mg to about 115 mg, about 70 mg to about 110 mg, about 70 mg to about 105 mg, about 70 mg to about 100 mg, about 70 mg to about 95 mg, about 70 mg to about 90 mg, about 70 mg to about 85 mg, about 70 mg to about 80 mg, about 75 mg to about 150 mg, about 75 mg to about 145 mg, about 75 mg to about 140 mg, about 75 mg to about 135 mg, about 75 mg to about 130 mg, about 75 mg to about 125 mg, about 75 mg to about 120 mg, about 75 mg to about 115 mg, about 75 mg to about 110 mg, about 75 mg to about 105 mg, about 75 mg to about 100 mg, about 75 mg to about 95 mg, about 75 mg to about 90 mg, about 75 mg to about 85 mg, about 80 mg to about 150 mg, about 80 mg to about 145 mg, about 80 mg to about 140 mg, about 80 mg to about 135 mg, about 80 mg to about 130 mg, about 80 mg to about 125 mg, about 80 mg to about 120 mg, about 80 mg to about 115 mg, about 80 mg to about 110 mg, about 80 mg to about 105 mg, about 80 mg to about 100 mg, about 80 mg to about 95 mg, about 80 mg to about 90 mg, about 85 mg to about 150 mg, about 85 mg to about 145 mg, about 85 mg to about 140 mg, about 85 mg to about 135 mg, about 85 mg to about 130 mg, about 85 mg to about 125 mg, about 85 mg to about 120 mg, about 85 mg to about 115 mg, about 85 mg to about 110 mg, about 85 mg to about 105 mg, about 85 mg to about 100 mg, about 85 mg to about 95 mg, about 90 mg to about 150 mg, about 90 mg to about 145 mg, about 90 mg to about 140 mg, about 90 mg to about 135 mg, about 90 mg to about 130 mg, about 90 mg to about 125 mg, about 90 mg to about 120 mg, about 90 mg to about 115 mg, about 90 mg to about 110 mg, about 90 mg to about 105 mg, about 90 mg to about 100 mg, about 95 mg to about 150 mg, about 95 mg to about 145 mg, about 95 mg to about 140 mg, about 95 mg to about 135 mg, about 95 mg to about 130 mg, about 95 mg to about 125 mg, about 95 mg to about 120 mg, about 95 mg to about 115 mg, about 95 mg to about 110 mg, about 95 mg to about 105 mg, about 100 mg to about 150 mg, about 100 mg to about 145 mg, about 100 mg to about 140 mg, about 100 mg to about 135 mg, about 100 mg to about 130 mg, about 100 mg to about 125 mg, about 100 mg to about 120 mg, about 100 mg to about 115 mg, about 100 mg to about 110 mg, about 105 mg to about 150 mg, about 105 mg to about 145 mg, about 105 mg to about 140 mg, about 105 mg to about 135 mg, about 105 mg to about 130 mg, about 105 mg to about 125 mg, about 105 mg to about 120 mg, about 105 mg to about 115 mg, about 110 mg to about 150 mg, about 110 mg to about 145 mg, about 110 mg to about 140 mg, about 110 mg to about 135 mg, about 110 mg to about 130 mg, about 110 mg to about 125 mg, about 110 mg to about 120 mg, about 115 mg to about 150 mg, about 115 mg to about 145 mg, about 115 mg to about 140 mg, about 115 mg to about 135 mg, about 115 mg to about 130 mg, about 115 mg to about 125 mg, about 120 mg to about 150 mg, about 120 mg to about 145 mg, about 120 mg to about 140 mg, about 120 mg to about 135 mg, about 120 mg to about 130 mg, about 125 mg to about 150 mg, about 125 mg to about 145 mg, about 125 mg to about 140 mg, about 125 mg to about 135 mg, about 130 mg to about 150 mg, about 130 mg to about 145 mg, about 130 mg to about 140 mg, about 135 mg to about 150 mg, about 135 mg to about 145 mg, or about 140 mg to about 150 mg) of a multi-chain chimeric polypeptide, wherein the time between administration of any two consecutive doses of the two or more doses is about 15 days to about 27 days (e.g., about 15 days to about 26 days, about 15 days to about 25 days, about 15 days to about 24 days, about 15 days to about 23 days, about 15 days to about 22 days, about 15 days to about 21 days, about 15 days to about 20 days, about 15 days to about 19 days, about 15 days to about 18 days, about 15 days to about 17 days, about 16 days to about 27 days, about 16 days to about 26 days, about 16 days to about 25 days, about 16 days to about 24 days, about 16 days to about 23 days, about 16 days to about 22 days, about 16 days to about 21 days, about 16 days to about 20 days, about 16 days to about 19 days, about 16 days to about 18 days, about 17 days to about 27 days, about 17 days to about 26 days, about 17 days to about 25 days, about 17 days to about 24 days, about 17 days to about 23 days, about 17 days to about 22 days, about 17 days to about 21 days, about 17 days to about 20 days, about 17 days to about 19 days, about 18 days to about 27 days, about 18 days to about 26 days, about 18 days to about 25 days, about 18 days to about 24 days, about 18 days to about 23 days, about 18 days to about 22 days, about 18 days to about 21 days, about 18 days to about 20 days, about 19 days to about 27 days, about 19 days to about 26 days, about 19 days to about 25 days, about 19 days to about 24 days, about 19 days to about 23 days, about 19 days to about 22 days, about 19 days to about 21 days, about 20 days to about 27 days, about 20 days to about 26 days, about 20 days to about 25 days, about 20 days to about 24 days, about 20 days to about 23 days, about 20 days to about 22 days, about 21 days to about 27 days, about 21 days to about 26 days, about 21 days to about 25 days, about 21 days to about 24 days, about 21 days to about 23 days, about 22 days to about 27 days, about 22 days to about 26 days, about 22 days to about 25 days, about 22 days to about 24 days, about 23 days to about 27 days, about 23 days to about 26 days, about 23 days to about 25 days, about 24 days to about 27 days, about 24 days to about 26 days, or about 25 days to about 27 days), where the multi-chain chimeric polypeptide comprises: (a) a first chimeric polypeptide comprising: (i) a first target-binding domain; (ii) a soluble tissue factor domain; and (iii) a first domain of a pair of affinity domains; and (b) a second chimeric polypeptide comprising: (i) a second domain of a pair of affinity domains; and (ii) a second target-binding domain, where: the first chimeric polypeptide and the second chimeric polypeptide associate through the binding of the first domain and the second domain of the pair of affinity domains; and the first target-binding domain binds specifically to a ligand of TGF-β receptor II (TGF-βRII) and the second target-binding domain binds specifically to a ligand of TGF-βRII. Any of the exemplary multi-chain chimeric polypeptides described herein can be used in these methods. Any of the exemplary multi-chain chimeric polypeptides described herein can be used in these methods.

In some embodiments, the subject has previously received a previous treatment with a standard first or later line of systemic therapy for a solid tumor, and the subject's solid tumor had recurred or progressed and/or the subject was intolerant to the previous treatment.

In some embodiments, the subject has been previously identified or diagnosed as having an unresectable, advanced, recurrent, and/or metastatic solid tumor or unresectable, advanced, recurrent, and/or metastatic solid cancer.

In some embodiments, the time between administration of any two consecutive doses of the two or more doses is about 18 days to about 24 days (e.g., about 18 days to about 23 days, about 18 days to about 22 days, about 18 days to about 21 days, about 18 days to about 20 days, about 19 days to about 24 days, about 19 days to about 23 days, about 19 days to about 22 days, about 19 days to about 21 days, about 20 days to about 24 days, about 20 days to about 23 days, about 20 days to about 22 days, about 21 days to about 24 days, about 21 days to about 23 days, or about 22 days to about 24 days).

In some embodiments, the time between administration of any two consecutive doses of the two or more doses is about 19 days to about 23 days (e.g., about 19 days to about 22 days, about 19 days to about 21 days, about 20 days to about 23 days, about 20 days to about 22 days, or about 21 days to about 23 days).

In some embodiments, the time between administration of any two consecutive doses of the two or more doses is about 20 days to about 22 days.

In some embodiments, the time between administration of any two consecutive doses of the two or more doses is about 21 days.

In some embodiments, each of the two or more doses is about 0.1 mg/kg to about 0.3 mg/kg (e.g., about 0.1 mg/kg to about 0.25 mg/kg, about 0.1 mg/kg to about 0.2 mg/kg, about 0.15 mg/kg to about 0.3 mg/kg, about 0.15 mg/kg to about 0.25 mg/kg, or about 0.2 mg/kg to about 0.3 mg/kg). In some embodiments, each of the two or more doses is about 0.25 mg/kg.

In some embodiments, each of the two or more doses is about 0.4 mg/kg to about 0.6 mg/kg (e.g., about 0.4 mg/kg to about 0.55 mg/kg, about 0.4 mg/kg to about 0.5 mg/kg, about 0.45 mg/kg to about 0.6 mg/kg, about 0.45 mg/kg to about 0.55 mg/kg, or about 0.5 mg/kg to about 0.6 mg/kg). In some embodiments, each of the two or more doses is about 0.5 mg/kg.

In some embodiments, each of the two or more doses is about 0.7 mg/kg to about 0.9 mg/kg (e.g., about 0.7 mg/kg to about 0.85 mg/kg, about 0.7 mg/kg to about 0.8 mg/kg, about 0.75 mg/kg to about 0.9 mg/kg, about 0.75 mg/kg to about 0.85 mg/kg, or about 0.8 mg/kg to about 0.9 mg/kg). In some embodiments, each of the two or more doses is about 0.8 mg/kg.

In some embodiments, each of the two or more doses is about 1.1 mg/kg to about 1.3 mg/kg (e.g., about 1.1 mg/kg to about 1.25 mg/kg, about 1.1 mg/kg to about 1.2 mg/kg, about 1.15 mg/kg to about 1.3 mg/kg, about 1.15 mg/kg to about 1.25 mg/kg, or about 1.2 mg/kg to about 1.3 mg/kg). In some embodiments, each of the two or more doses is about 1.2 mg/kg.

In some embodiments, each of the two or more doses is about 0.25 mg/kg and the time between administration of any two consecutive doses of the two or more doses is about 21 days.

In some embodiments, each of the two or more doses is about 0.5 mg/kg and the time between administration of any two consecutive doses of the two or more doses is about 21 days.

In some embodiments, each of the two or more doses is about 0.8 mg/kg and the time between administration of any two consecutive doses of the two or more doses is about 21 days.

In some embodiments, each of the two or more doses is about 1.2 mg/kg and the time between administration of any two consecutive doses of the two or more doses is about 21 days.

In some embodiments, each of the two or more doses is about 25 mg to about 150 mg (e.g., about 25 mg to about 145 mg, about 25 mg to about 140 mg, about 25 mg to about 135 mg, about 25 mg to about 130 mg, about 25 mg to about 125 mg, about 25 mg to about 120 mg, about 25 mg to about 115 mg, about 25 mg to about 110 mg, about 25 mg to about 105 mg, about 25 mg to about 100 mg, about 25 mg to about 95 mg, about 25 mg to about 90 mg, about 25 mg to about 85 mg, about 25 mg to about 80 mg, about 25 mg to about 75 mg, about 25 mg to about 70 mg, about 25 mg to about 65 mg, about 25 mg to about 60 mg, about 25 mg to about 55 mg, about 25 mg to about 50 mg, about 25 mg to about 45 mg, about 25 mg to about 40 mg, about 25 mg to about 35 mg, about 30 mg to about 150 mg, about 30 mg to about 145 mg, about 30 mg to about 140 mg, about 30 mg to about 135 mg, about 30 mg to about 130 mg, about 30 mg to about 125 mg, about 30 mg to about 120 mg, about 30 mg to about 115 mg, about 30 mg to about 110 mg, about 30 mg to about 105 mg, about 30 mg to about 100 mg, about 30 mg to about 95 mg, about 30 mg to about 90 mg, about 30 mg to about 85 mg, about 30 mg to about 80 mg, about 30 mg to about 75 mg, about 30 mg to about 70 mg, about 30 mg to about 65 mg, about 30 mg to about 60 mg, about 30 mg to about 55 mg, about 30 mg to about 50 mg, about 30 mg to about 45 mg, about 30 mg to about 40 mg, about 35 mg to about 150 mg, about 35 mg to about 145 mg, about 35 mg to about 140 mg, about 35 mg to about 135 mg, about 35 mg to about 130 mg, about 35 mg to about 125 mg, about 35 mg to about 120 mg, about 35 mg to about 115 mg, about 35 mg to about 110 mg, about 35 mg to about 105 mg, about 35 mg to about 100 mg, about 35 mg to about 95 mg, about 35 mg to about 90 mg, about 35 mg to about 85 mg, about 35 mg to about 80 mg, about 35 mg to about 75 mg, about 35 mg to about 70 mg, about 35 mg to about 65 mg, about 35 mg to about 60 mg, about 35 mg to about 55 mg, about 35 mg to about 50 mg, about 35 mg to about 45 mg, about 40 mg to about 150 mg, about 40 mg to about 145 mg, about 40 mg to about 140 mg, about 40 mg to about 135 mg, about 40 mg to about 130 mg, about 40 mg to about 125 mg, about 40 mg to about 120 mg, about 40 mg to about 115 mg, about 40 mg to about 110 mg, about 40 mg to about 105 mg, about 40 mg to about 100 mg, about 40 mg to about 95 mg, about 40 mg to about 90 mg, about 40 mg to about 85 mg, about 40 mg to about 80 mg, about 40 mg to about 75 mg, about 40 mg to about 70 mg, about 40 mg to about 65 mg, about 40 mg to about 60 mg, about 40 mg to about 55 mg, about 40 mg to about 50 mg, about 45 mg to about 150 mg, about 45 mg to about 145 mg, about 45 mg to about 140 mg, about 45 mg to about 135 mg, about 45 mg to about 130 mg, about 45 mg to about 125 mg, about 45 mg to about 120 mg, about 45 mg to about 115 mg, about 45 mg to about 110 mg, about 45 mg to about 105 mg, about 45 mg to about 100 mg, about 45 mg to about 95 mg, about 45 mg to about 90 mg, about 45 mg to about 85 mg, about 45 mg to about 80 mg, about 45 mg to about 75 mg, about 45 mg to about 70 mg, about 45 mg to about 65 mg, about 45 mg to about 60 mg, about 45 mg to about 55 mg, about 50 mg to about 150 mg, about 50 mg to about 145 mg, about 50 mg to about 140 mg, about 50 mg to about 135 mg, about 50 mg to about 130 mg, about 50 mg to about 125 mg, about 50 mg to about 120 mg, about 50 mg to about 115 mg, about 50 mg to about 110 mg, about 50 mg to about 105 mg, about 50 mg to about 100 mg, about 50 mg to about 95 mg, about 50 mg to about 90 mg, about 50 mg to about 85 mg, about 50 mg to about 80 mg, about 50 mg to about 75 mg, about 50 mg to about 70 mg, about 50 mg to about 65 mg, about 50 mg to about 60 mg, about 55 mg to about 150 mg, about 55 mg to about 145 mg, about 55 mg to about 140 mg, about 55 mg to about 135 mg, about 55 mg to about 130 mg, about 55 mg to about 125 mg, about 55 mg to about 120 mg, about 55 mg to about 115 mg, about 55 mg to about 110 mg, about 55 mg to about 105 mg, about 55 mg to about 100 mg, about 55 mg to about 95 mg, about 55 mg to about 90 mg, about 55 mg to about 85 mg, about 55 mg to about 80 mg, about 55 mg to about 75 mg, about 55 mg to about 70 mg, about 55 mg to about 65 mg, about 60 mg to about 150 mg, about 60 mg to about 145 mg, about 60 mg to about 140 mg, about 60 mg to about 135 mg, about 60 mg to about 130 mg, about 60 mg to about 125 mg, about 60 mg to about 120 mg, about 60 mg to about 115 mg, about 60 mg to about 110 mg, about 60 mg to about 105 mg, about 60 mg to about 100 mg, about 60 mg to about 95 mg, about 60 mg to about 90 mg, about 60 mg to about 85 mg, about 60 mg to about 80 mg, about 60 mg to about 75 mg, about 60 mg to about 70 mg, about 65 mg to about 150 mg, about 65 mg to about 145 mg, about 65 mg to about 140 mg, about 65 mg to about 135 mg, about 65 mg to about 130 mg, about 65 mg to about 125 mg, about 65 mg to about 120 mg, about 65 mg to about 115 mg, about 65 mg to about 110 mg, about 65 mg to about 105 mg, about 65 mg to about 100 mg, about 65 mg to about 95 mg, about 65 mg to about 90 mg, about 65 mg to about 85 mg, about 65 mg to about 80 mg, about 65 mg to about 75 mg, about 70 mg to about 150 mg, about 70 mg to about 145 mg, about 70 mg to about 140 mg, about 70 mg to about 135 mg, about 70 mg to about 130 mg, about 70 mg to about 125 mg, about 70 mg to about 120 mg, about 70 mg to about 115 mg, about 70 mg to about 110 mg, about 70 mg to about 105 mg, about 70 mg to about 100 mg, about 70 mg to about 95 mg, about 70 mg to about 90 mg, about 70 mg to about 85 mg, about 70 mg to about 80 mg, about 75 mg to about 150 mg, about 75 mg to about 145 mg, about 75 mg to about 140 mg, about 75 mg to about 135 mg, about 75 mg to about 130 mg, about 75 mg to about 125 mg, about 75 mg to about 120 mg, about 75 mg to about 115 mg, about 75 mg to about 110 mg, about 75 mg to about 105 mg, about 75 mg to about 100 mg, about 75 mg to about 95 mg, about 75 mg to about 90 mg, about 75 mg to about 85 mg, about 80 mg to about 150 mg, about 80 mg to about 145 mg, about 80 mg to about 140 mg, about 80 mg to about 135 mg, about 80 mg to about 130 mg, about 80 mg to about 125 mg, about 80 mg to about 120 mg, about 80 mg to about 115 mg, about 80 mg to about 110 mg, about 80 mg to about 105 mg, about 80 mg to about 100 mg, about 80 mg to about 95 mg, about 80 mg to about 90 mg, about 85 mg to about 150 mg, about 85 mg to about 145 mg, about 85 mg to about 140 mg, about 85 mg to about 135 mg, about 85 mg to about 130 mg, about 85 mg to about 125 mg, about 85 mg to about 120 mg, about 85 mg to about 115 mg, about 85 mg to about 110 mg, about 85 mg to about 105 mg, about 85 mg to about 100 mg, about 85 mg to about 95 mg, about 90 mg to about 150 mg, about 90 mg to about 145 mg, about 90 mg to about 140 mg, about 90 mg to about 135 mg, about 90 mg to about 130 mg, about 90 mg to about 125 mg, about 90 mg to about 120 mg, about 90 mg to about 115 mg, about 90 mg to about 110 mg, about 90 mg to about 105 mg, about 90 mg to about 100 mg, about 95 mg to about 150 mg, about 95 mg to about 145 mg, about 95 mg to about 140 mg, about 95 mg to about 135 mg, about 95 mg to about 130 mg, about 95 mg to about 125 mg, about 95 mg to about 120 mg, about 95 mg to about 115 mg, about 95 mg to about 110 mg, about 95 mg to about 105 mg, about 100 mg to about 150 mg, about 100 mg to about 145 mg, about 100 mg to about 140 mg, about 100 mg to about 135 mg, about 100 mg to about 130 mg, about 100 mg to about 125 mg, about 100 mg to about 120 mg, about 100 mg to about 115 mg, about 100 mg to about 110 mg, about 105 mg to about 150 mg, about 105 mg to about 145 mg, about 105 mg to about 140 mg, about 105 mg to about 135 mg, about 105 mg to about 130 mg, about 105 mg to about 125 mg, about 105 mg to about 120 mg, about 105 mg to about 115 mg, about 110 mg to about 150 mg, about 110 mg to about 145 mg, about 110 mg to about 140 mg, about 110 mg to about 135 mg, about 110 mg to about 130 mg, about 110 mg to about 125 mg, about 110 mg to about 120 mg, about 115 mg to about 150 mg, about 115 mg to about 145 mg, about 115 mg to about 140 mg, about 115 mg to about 135 mg, about 115 mg to about 130 mg, about 115 mg to about 125 mg, about 120 mg to about 150 mg, about 120 mg to about 145 mg, about 120 mg to about 140 mg, about 120 mg to about 135 mg, about 120 mg to about 130 mg, about 125 mg to about 150 mg, about 125 mg to about 145 mg, about 125 mg to about 140 mg, about 125 mg to about 135 mg, about 130 mg to about 150 mg, about 130 mg to about 145 mg, about 130 mg to about 140 mg, about 135 mg to about 150 mg, about 135 mg to about 145 mg, or about 140 mg to about 150 mg). In some embodiments, the time between administration of any two consecutive doses of the two or more doses is about 21 days.

In some embodiments, each of the two or more doses is about 35 mg to about 150 mg (e.g., about 35 mg to about 145 mg, about 35 mg to about 140 mg, about 35 mg to about 135 mg, about 35 mg to about 130 mg, about 35 mg to about 125 mg, about 35 mg to about 120 mg, about 35 mg to about 115 mg, about 35 mg to about 110 mg, about 35 mg to about 105 mg, about 35 mg to about 100 mg, about 35 mg to about 95 mg, about 35 mg to about 90 mg, about 35 mg to about 85 mg, about 35 mg to about 80 mg, about 35 mg to about 75 mg, about 35 mg to about 70 mg, about 35 mg to about 65 mg, about 35 mg to about 60 mg, about 35 mg to about 55 mg, about 35 mg to about 50 mg, about 35 mg to about 45 mg, about 40 mg to about 150 mg, about 40 mg to about 145 mg, about 40 mg to about 140 mg, about 40 mg to about 135 mg, about 40 mg to about 130 mg, about 40 mg to about 125 mg, about 40 mg to about 120 mg, about 40 mg to about 115 mg, about 40 mg to about 110 mg, about 40 mg to about 105 mg, about 40 mg to about 100 mg, about 40 mg to about 95 mg, about 40 mg to about 90 mg, about 40 mg to about 85 mg, about 40 mg to about 80 mg, about 40 mg to about 75 mg, about 40 mg to about 70 mg, about 40 mg to about 65 mg, about 40 mg to about 60 mg, about 40 mg to about 55 mg, about 40 mg to about 50 mg, about 45 mg to about 150 mg, about 45 mg to about 145 mg, about 45 mg to about 140 mg, about 45 mg to about 135 mg, about 45 mg to about 130 mg, about 45 mg to about 125 mg, about 45 mg to about 120 mg, about 45 mg to about 115 mg, about 45 mg to about 110 mg, about 45 mg to about 105 mg, about 45 mg to about 100 mg, about 45 mg to about 95 mg, about 45 mg to about 90 mg, about 45 mg to about 85 mg, about 45 mg to about 80 mg, about 45 mg to about 75 mg, about 45 mg to about 70 mg, about 45 mg to about 65 mg, about 45 mg to about 60 mg, about 45 mg to about 55 mg, about 50 mg to about 150 mg, about 50 mg to about 145 mg, about 50 mg to about 140 mg, about 50 mg to about 135 mg, about 50 mg to about 130 mg, about 50 mg to about 125 mg, about 50 mg to about 120 mg, about 50 mg to about 115 mg, about 50 mg to about 110 mg, about 50 mg to about 105 mg, about 50 mg to about 100 mg, about 50 mg to about 95 mg, about 50 mg to about 90 mg, about 50 mg to about 85 mg, about 50 mg to about 80 mg, about 50 mg to about 75 mg, about 50 mg to about 70 mg, about 50 mg to about 65 mg, about 50 mg to about 60 mg, about 55 mg to about 150 mg, about 55 mg to about 145 mg, about 55 mg to about 140 mg, about 55 mg to about 135 mg, about 55 mg to about 130 mg, about 55 mg to about 125 mg, about 55 mg to about 120 mg, about 55 mg to about 115 mg, about 55 mg to about 110 mg, about 55 mg to about 105 mg, about 55 mg to about 100 mg, about 55 mg to about 95 mg, about 55 mg to about 90 mg, about 55 mg to about 85 mg, about 55 mg to about 80 mg, about 55 mg to about 75 mg, about 55 mg to about 70 mg, about 55 mg to about 65 mg, about 60 mg to about 150 mg, about 60 mg to about 145 mg, about 60 mg to about 140 mg, about 60 mg to about 135 mg, about 60 mg to about 130 mg, about 60 mg to about 125 mg, about 60 mg to about 120 mg, about 60 mg to about 115 mg, about 60 mg to about 110 mg, about 60 mg to about 105 mg, about 60 mg to about 100 mg, about 60 mg to about 95 mg, about 60 mg to about 90 mg, about 60 mg to about 85 mg, about 60 mg to about 80 mg, about 60 mg to about 75 mg, about 60 mg to about 70 mg, about 65 mg to about 150 mg, about 65 mg to about 145 mg, about 65 mg to about 140 mg, about 65 mg to about 135 mg, about 65 mg to about 130 mg, about 65 mg to about 125 mg, about 65 mg to about 120 mg, about 65 mg to about 115 mg, about 65 mg to about 110 mg, about 65 mg to about 105 mg, about 65 mg to about 100 mg, about 65 mg to about 95 mg, about 65 mg to about 90 mg, about 65 mg to about 85 mg, about 65 mg to about 80 mg, about 65 mg to about 75 mg, about 70 mg to about 150 mg, about 70 mg to about 145 mg, about 70 mg to about 140 mg, about 70 mg to about 135 mg, about 70 mg to about 130 mg, about 70 mg to about 125 mg, about 70 mg to about 120 mg, about 70 mg to about 115 mg, about 70 mg to about 110 mg, about 70 mg to about 105 mg, about 70 mg to about 100 mg, about 70 mg to about 95 mg, about 70 mg to about 90 mg, about 70 mg to about 85 mg, about 70 mg to about 80 mg, about 75 mg to about 150 mg, about 75 mg to about 145 mg, about 75 mg to about 140 mg, about 75 mg to about 135 mg, about 75 mg to about 130 mg, about 75 mg to about 125 mg, about 75 mg to about 120 mg, about 75 mg to about 115 mg, about 75 mg to about 110 mg, about 75 mg to about 105 mg, about 75 mg to about 100 mg, about 75 mg to about 95 mg, about 75 mg to about 90 mg, about 75 mg to about 85 mg, about 80 mg to about 150 mg, about 80 mg to about 145 mg, about 80 mg to about 140 mg, about 80 mg to about 135 mg, about 80 mg to about 130 mg, about 80 mg to about 125 mg, about 80 mg to about 120 mg, about 80 mg to about 115 mg, about 80 mg to about 110 mg, about 80 mg to about 105 mg, about 80 mg to about 100 mg, about 80 mg to about 95 mg, about 80 mg to about 90 mg, about 85 mg to about 150 mg, about 85 mg to about 145 mg, about 85 mg to about 140 mg, about 85 mg to about 135 mg, about 85 mg to about 130 mg, about 85 mg to about 125 mg, about 85 mg to about 120 mg, about 85 mg to about 115 mg, about 85 mg to about 110 mg, about 85 mg to about 105 mg, about 85 mg to about 100 mg, about 85 mg to about 95 mg, about 90 mg to about 150 mg, about 90 mg to about 145 mg, about 90 mg to about 140 mg, about 90 mg to about 135 mg, about 90 mg to about 130 mg, about 90 mg to about 125 mg, about 90 mg to about 120 mg, about 90 mg to about 115 mg, about 90 mg to about 110 mg, about 90 mg to about 105 mg, about 90 mg to about 100 mg, about 95 mg to about 150 mg, about 95 mg to about 145 mg, about 95 mg to about 140 mg, about 95 mg to about 135 mg, about 95 mg to about 130 mg, about 95 mg to about 125 mg, about 95 mg to about 120 mg, about 95 mg to about 115 mg, about 95 mg to about 110 mg, about 95 mg to about 105 mg, about 100 mg to about 150 mg, about 100 mg to about 145 mg, about 100 mg to about 140 mg, about 100 mg to about 135 mg, about 100 mg to about 130 mg, about 100 mg to about 125 mg, about 100 mg to about 120 mg, about 100 mg to about 115 mg, about 100 mg to about 110 mg, about 105 mg to about 150 mg, about 105 mg to about 145 mg, about 105 mg to about 140 mg, about 105 mg to about 135 mg, about 105 mg to about 130 mg, about 105 mg to about 125 mg, about 105 mg to about 120 mg, about 105 mg to about 115 mg, about 110 mg to about 150 mg, about 110 mg to about 145 mg, about 110 mg to about 140 mg, about 110 mg to about 135 mg, about 110 mg to about 130 mg, about 110 mg to about 125 mg, about 110 mg to about 120 mg, about 115 mg to about 150 mg, about 115 mg to about 145 mg, about 115 mg to about 140 mg, about 115 mg to about 135 mg, about 115 mg to about 130 mg, about 115 mg to about 125 mg, about 120 mg to about 150 mg, about 120 mg to about 145 mg, about 120 mg to about 140 mg, about 120 mg to about 135 mg, about 120 mg to about 130 mg, about 125 mg to about 150 mg, about 125 mg to about 145 mg, about 125 mg to about 140 mg, about 125 mg to about 135 mg, about 130 mg to about 150 mg, about 130 mg to about 145 mg, about 130 mg to about 140 mg, about 135 mg to about 150 mg, about 135 mg to about 145 mg, or about 140 mg to about 150 mg). In some embodiments, the time between administration of any two consecutive doses of the two or more doses is about 21 days.

In some embodiments, each of the two or more doses is about 40 mg to about 150 mg (e.g., about 40 mg to about 145 mg, about 40 mg to about 140 mg, about 40 mg to about 135 mg, about 40 mg to about 130 mg, about 40 mg to about 125 mg, about 40 mg to about 120 mg, about 40 mg to about 115 mg, about 40 mg to about 110 mg, about 40 mg to about 105 mg, about 40 mg to about 100 mg, about 40 mg to about 95 mg, about 40 mg to about 90 mg, about 40 mg to about 85 mg, about 40 mg to about 80 mg, about 40 mg to about 75 mg, about 40 mg to about 70 mg, about 40 mg to about 65 mg, about 40 mg to about 60 mg, about 40 mg to about 55 mg, about 40 mg to about 50 mg, about 45 mg to about 150 mg, about 45 mg to about 145 mg, about 45 mg to about 140 mg, about 45 mg to about 135 mg, about 45 mg to about 130 mg, about 45 mg to about 125 mg, about 45 mg to about 120 mg, about 45 mg to about 115 mg, about 45 mg to about 110 mg, about 45 mg to about 105 mg, about 45 mg to about 100 mg, about 45 mg to about 95 mg, about 45 mg to about 90 mg, about 45 mg to about 85 mg, about 45 mg to about 80 mg, about 45 mg to about 75 mg, about 45 mg to about 70 mg, about 45 mg to about 65 mg, about 45 mg to about 60 mg, about 45 mg to about 55 mg, about 50 mg to about 150 mg, about 50 mg to about 145 mg, about 50 mg to about 140 mg, about 50 mg to about 135 mg, about 50 mg to about 130 mg, about 50 mg to about 125 mg, about 50 mg to about 120 mg, about 50 mg to about 115 mg, about 50 mg to about 110 mg, about 50 mg to about 105 mg, about 50 mg to about 100 mg, about 50 mg to about 95 mg, about 50 mg to about 90 mg, about 50 mg to about 85 mg, about 50 mg to about 80 mg, about 50 mg to about 75 mg, about 50 mg to about 70 mg, about 50 mg to about 65 mg, about 50 mg to about 60 mg, about 55 mg to about 150 mg, about 55 mg to about 145 mg, about 55 mg to about 140 mg, about 55 mg to about 135 mg, about 55 mg to about 130 mg, about 55 mg to about 125 mg, about 55 mg to about 120 mg, about 55 mg to about 115 mg, about 55 mg to about 110 mg, about 55 mg to about 105 mg, about 55 mg to about 100 mg, about 55 mg to about 95 mg, about 55 mg to about 90 mg, about 55 mg to about 85 mg, about 55 mg to about 80 mg, about 55 mg to about 75 mg, about 55 mg to about 70 mg, about 55 mg to about 65 mg, about 60 mg to about 150 mg, about 60 mg to about 145 mg, about 60 mg to about 140 mg, about 60 mg to about 135 mg, about 60 mg to about 130 mg, about 60 mg to about 125 mg, about 60 mg to about 120 mg, about 60 mg to about 115 mg, about 60 mg to about 110 mg, about 60 mg to about 105 mg, about 60 mg to about 100 mg, about 60 mg to about 95 mg, about 60 mg to about 90 mg, about 60 mg to about 85 mg, about 60 mg to about 80 mg, about 60 mg to about 75 mg, about 60 mg to about 70 mg, about 65 mg to about 150 mg, about 65 mg to about 145 mg, about 65 mg to about 140 mg, about 65 mg to about 135 mg, about 65 mg to about 130 mg, about 65 mg to about 125 mg, about 65 mg to about 120 mg, about 65 mg to about 115 mg, about 65 mg to about 110 mg, about 65 mg to about 105 mg, about 65 mg to about 100 mg, about 65 mg to about 95 mg, about 65 mg to about 90 mg, about 65 mg to about 85 mg, about 65 mg to about 80 mg, about 65 mg to about 75 mg, about 70 mg to about 150 mg, about 70 mg to about 145 mg, about 70 mg to about 140 mg, about 70 mg to about 135 mg, about 70 mg to about 130 mg, about 70 mg to about 125 mg, about 70 mg to about 120 mg, about 70 mg to about 115 mg, about 70 mg to about 110 mg, about 70 mg to about 105 mg, about 70 mg to about 100 mg, about 70 mg to about 95 mg, about 70 mg to about 90 mg, about 70 mg to about 85 mg, about 70 mg to about 80 mg, about 75 mg to about 150 mg, about 75 mg to about 145 mg, about 75 mg to about 140 mg, about 75 mg to about 135 mg, about 75 mg to about 130 mg, about 75 mg to about 125 mg, about 75 mg to about 120 mg, about 75 mg to about 115 mg, about 75 mg to about 110 mg, about 75 mg to about 105 mg, about 75 mg to about 100 mg, about 75 mg to about 95 mg, about 75 mg to about 90 mg, about 75 mg to about 85 mg, about 80 mg to about 150 mg, about 80 mg to about 145 mg, about 80 mg to about 140 mg, about 80 mg to about 135 mg, about 80 mg to about 130 mg, about 80 mg to about 125 mg, about 80 mg to about 120 mg, about 80 mg to about 115 mg, about 80 mg to about 110 mg, about 80 mg to about 105 mg, about 80 mg to about 100 mg, about 80 mg to about 95 mg, about 80 mg to about 90 mg, about 85 mg to about 150 mg, about 85 mg to about 145 mg, about 85 mg to about 140 mg, about 85 mg to about 135 mg, about 85 mg to about 130 mg, about 85 mg to about 125 mg, about 85 mg to about 120 mg, about 85 mg to about 115 mg, about 85 mg to about 110 mg, about 85 mg to about 105 mg, about 85 mg to about 100 mg, about 85 mg to about 95 mg, about 90 mg to about 150 mg, about 90 mg to about 145 mg, about 90 mg to about 140 mg, about 90 mg to about 135 mg, about 90 mg to about 130 mg, about 90 mg to about 125 mg, about 90 mg to about 120 mg, about 90 mg to about 115 mg, about 90 mg to about 110 mg, about 90 mg to about 105 mg, about 90 mg to about 100 mg, about 95 mg to about 150 mg, about 95 mg to about 145 mg, about 95 mg to about 140 mg, about 95 mg to about 135 mg, about 95 mg to about 130 mg, about 95 mg to about 125 mg, about 95 mg to about 120 mg, about 95 mg to about 115 mg, about 95 mg to about 110 mg, about 95 mg to about 105 mg, about 100 mg to about 150 mg, about 100 mg to about 145 mg, about 100 mg to about 140 mg, about 100 mg to about 135 mg, about 100 mg to about 130 mg, about 100 mg to about 125 mg, about 100 mg to about 120 mg, about 100 mg to about 115 mg, about 100 mg to about 110 mg, about 105 mg to about 150 mg, about 105 mg to about 145 mg, about 105 mg to about 140 mg, about 105 mg to about 135 mg, about 105 mg to about 130 mg, about 105 mg to about 125 mg, about 105 mg to about 120 mg, about 105 mg to about 115 mg, about 110 mg to about 150 mg, about 110 mg to about 145 mg, about 110 mg to about 140 mg, about 110 mg to about 135 mg, about 110 mg to about 130 mg, about 110 mg to about 125 mg, about 110 mg to about 120 mg, about 115 mg to about 150 mg, about 115 mg to about 145 mg, about 115 mg to about 140 mg, about 115 mg to about 135 mg, about 115 mg to about 130 mg, about 115 mg to about 125 mg, about 120 mg to about 150 mg, about 120 mg to about 145 mg, about 120 mg to about 140 mg, about 120 mg to about 135 mg, about 120 mg to about 130 mg, about 125 mg to about 150 mg, about 125 mg to about 145 mg, about 125 mg to about 140 mg, about 125 mg to about 135 mg, about 130 mg to about 150 mg, about 130 mg to about 145 mg, about 130 mg to about 140 mg, about 135 mg to about 150 mg, about 135 mg to about 145 mg, or about 140 mg to about 150 mg). In some embodiments, the time between administration of any two consecutive doses of the two or more doses is about 21 days.

In some embodiments, each of the two or more doses is about 50 mg to about 150 mg (e.g., about 50 mg to about 145 mg, about 50 mg to about 140 mg, about 50 mg to about 135 mg, about 50 mg to about 130 mg, about 50 mg to about 125 mg, about 50 mg to about 120 mg, about 50 mg to about 115 mg, about 50 mg to about 110 mg, about 50 mg to about 105 mg, about 50 mg to about 100 mg, about 50 mg to about 95 mg, about 50 mg to about 90 mg, about 50 mg to about 85 mg, about 50 mg to about 80 mg, about 50 mg to about 75 mg, about 50 mg to about 70 mg, about 50 mg to about 65 mg, about 50 mg to about 60 mg, about 55 mg to about 150 mg, about 55 mg to about 145 mg, about 55 mg to about 140 mg, about 55 mg to about 135 mg, about 55 mg to about 130 mg, about 55 mg to about 125 mg, about 55 mg to about 120 mg, about 55 mg to about 115 mg, about 55 mg to about 110 mg, about 55 mg to about 105 mg, about 55 mg to about 100 mg, about 55 mg to about 95 mg, about 55 mg to about 90 mg, about 55 mg to about 85 mg, about 55 mg to about 80 mg, about 55 mg to about 75 mg, about 55 mg to about 70 mg, about 55 mg to about 65 mg, about 60 mg to about 150 mg, about 60 mg to about 145 mg, about 60 mg to about 140 mg, about 60 mg to about 135 mg, about 60 mg to about 130 mg, about 60 mg to about 125 mg, about 60 mg to about 120 mg, about 60 mg to about 115 mg, about 60 mg to about 110 mg, about 60 mg to about 105 mg, about 60 mg to about 100 mg, about 60 mg to about 95 mg, about 60 mg to about 90 mg, about 60 mg to about 85 mg, about 60 mg to about 80 mg, about 60 mg to about 75 mg, about 60 mg to about 70 mg, about 65 mg to about 150 mg, about 65 mg to about 145 mg, about 65 mg to about 140 mg, about 65 mg to about 135 mg, about 65 mg to about 130 mg, about 65 mg to about 125 mg, about 65 mg to about 120 mg, about 65 mg to about 115 mg, about 65 mg to about 110 mg, about 65 mg to about 105 mg, about 65 mg to about 100 mg, about 65 mg to about 95 mg, about 65 mg to about 90 mg, about 65 mg to about 85 mg, about 65 mg to about 80 mg, about 65 mg to about 75 mg, about 70 mg to about 150 mg, about 70 mg to about 145 mg, about 70 mg to about 140 mg, about 70 mg to about 135 mg, about 70 mg to about 130 mg, about 70 mg to about 125 mg, about 70 mg to about 120 mg, about 70 mg to about 115 mg, about 70 mg to about 110 mg, about 70 mg to about 105 mg, about 70 mg to about 100 mg, about 70 mg to about 95 mg, about 70 mg to about 90 mg, about 70 mg to about 85 mg, about 70 mg to about 80 mg, about 75 mg to about 150 mg, about 75 mg to about 145 mg, about 75 mg to about 140 mg, about 75 mg to about 135 mg, about 75 mg to about 130 mg, about 75 mg to about 125 mg, about 75 mg to about 120 mg, about 75 mg to about 115 mg, about 75 mg to about 110 mg, about 75 mg to about 105 mg, about 75 mg to about 100 mg, about 75 mg to about 95 mg, about 75 mg to about 90 mg, about 75 mg to about 85 mg, about 80 mg to about 150 mg, about 80 mg to about 145 mg, about 80 mg to about 140 mg, about 80 mg to about 135 mg, about 80 mg to about 130 mg, about 80 mg to about 125 mg, about 80 mg to about 120 mg, about 80 mg to about 115 mg, about 80 mg to about 110 mg, about 80 mg to about 105 mg, about 80 mg to about 100 mg, about 80 mg to about 95 mg, about 80 mg to about 90 mg, about 85 mg to about 150 mg, about 85 mg to about 145 mg, about 85 mg to about 140 mg, about 85 mg to about 135 mg, about 85 mg to about 130 mg, about 85 mg to about 125 mg, about 85 mg to about 120 mg, about 85 mg to about 115 mg, about 85 mg to about 110 mg, about 85 mg to about 105 mg, about 85 mg to about 100 mg, about 85 mg to about 95 mg, about 90 mg to about 150 mg, about 90 mg to about 145 mg, about 90 mg to about 140 mg, about 90 mg to about 135 mg, about 90 mg to about 130 mg, about 90 mg to about 125 mg, about 90 mg to about 120 mg, about 90 mg to about 115 mg, about 90 mg to about 110 mg, about 90 mg to about 105 mg, about 90 mg to about 100 mg, about 95 mg to about 150 mg, about 95 mg to about 145 mg, about 95 mg to about 140 mg, about 95 mg to about 135 mg, about 95 mg to about 130 mg, about 95 mg to about 125 mg, about 95 mg to about 120 mg, about 95 mg to about 115 mg, about 95 mg to about 110 mg, about 95 mg to about 105 mg, about 100 mg to about 150 mg, about 100 mg to about 145 mg, about 100 mg to about 140 mg, about 100 mg to about 135 mg, about 100 mg to about 130 mg, about 100 mg to about 125 mg, about 100 mg to about 120 mg, about 100 mg to about 115 mg, about 100 mg to about 110 mg, about 105 mg to about 150 mg, about 105 mg to about 145 mg, about 105 mg to about 140 mg, about 105 mg to about 135 mg, about 105 mg to about 130 mg, about 105 mg to about 125 mg, about 105 mg to about 120 mg, about 105 mg to about 115 mg, about 110 mg to about 150 mg, about 110 mg to about 145 mg, about 110 mg to about 140 mg, about 110 mg to about 135 mg, about 110 mg to about 130 mg, about 110 mg to about 125 mg, about 110 mg to about 120 mg, about 115 mg to about 150 mg, about 115 mg to about 145 mg, about 115 mg to about 140 mg, about 115 mg to about 135 mg, about 115 mg to about 130 mg, about 115 mg to about 125 mg, about 120 mg to about 150 mg, about 120 mg to about 145 mg, about 120 mg to about 140 mg, about 120 mg to about 135 mg, about 120 mg to about 130 mg, about 125 mg to about 150 mg, about 125 mg to about 145 mg, about 125 mg to about 140 mg, about 125 mg to about 135 mg, about 130 mg to about 150 mg, about 130 mg to about 145 mg, about 130 mg to about 140 mg, about 135 mg to about 150 mg, about 135 mg to about 145 mg, or about 140 mg to about 150 mg). In some embodiments, the time between administration of any two consecutive doses of the two or more doses is about 21 days.

In some embodiments, each of the two or more doses is about 60 mg to about 150 mg (e.g., about 60 mg to about 145 mg, about 60 mg to about 140 mg, about 60 mg to about 135 mg, about 60 mg to about 130 mg, about 60 mg to about 125 mg, about 60 mg to about 120 mg, about 60 mg to about 115 mg, about 60 mg to about 110 mg, about 60 mg to about 105 mg, about 60 mg to about 100 mg, about 60 mg to about 95 mg, about 60 mg to about 90 mg, about 60 mg to about 85 mg, about 60 mg to about 80 mg, about 60 mg to about 75 mg, about 60 mg to about 70 mg, about 65 mg to about 150 mg, about 65 mg to about 145 mg, about 65 mg to about 140 mg, about 65 mg to about 135 mg, about 65 mg to about 130 mg, about 65 mg to about 125 mg, about 65 mg to about 120 mg, about 65 mg to about 115 mg, about 65 mg to about 110 mg, about 65 mg to about 105 mg, about 65 mg to about 100 mg, about 65 mg to about 95 mg, about 65 mg to about 90 mg, about 65 mg to about 85 mg, about 65 mg to about 80 mg, about 65 mg to about 75 mg, about 70 mg to about 150 mg, about 70 mg to about 145 mg, about 70 mg to about 140 mg, about 70 mg to about 135 mg, about 70 mg to about 130 mg, about 70 mg to about 125 mg, about 70 mg to about 120 mg, about 70 mg to about 115 mg, about 70 mg to about 110 mg, about 70 mg to about 105 mg, about 70 mg to about 100 mg, about 70 mg to about 95 mg, about 70 mg to about 90 mg, about 70 mg to about 85 mg, about 70 mg to about 80 mg, about 75 mg to about 150 mg, about 75 mg to about 145 mg, about 75 mg to about 140 mg, about 75 mg to about 135 mg, about 75 mg to about 130 mg, about 75 mg to about 125 mg, about 75 mg to about 120 mg, about 75 mg to about 115 mg, about 75 mg to about 110 mg, about 75 mg to about 105 mg, about 75 mg to about 100 mg, about 75 mg to about 95 mg, about 75 mg to about 90 mg, about 75 mg to about 85 mg, about 80 mg to about 150 mg, about 80 mg to about 145 mg, about 80 mg to about 140 mg, about 80 mg to about 135 mg, about 80 mg to about 130 mg, about 80 mg to about 125 mg, about 80 mg to about 120 mg, about 80 mg to about 115 mg, about 80 mg to about 110 mg, about 80 mg to about 105 mg, about 80 mg to about 100 mg, about 80 mg to about 95 mg, about 80 mg to about 90 mg, about 85 mg to about 150 mg, about 85 mg to about 145 mg, about 85 mg to about 140 mg, about 85 mg to about 135 mg, about 85 mg to about 130 mg, about 85 mg to about 125 mg, about 85 mg to about 120 mg, about 85 mg to about 115 mg, about 85 mg to about 110 mg, about 85 mg to about 105 mg, about 85 mg to about 100 mg, about 85 mg to about 95 mg, about 90 mg to about 150 mg, about 90 mg to about 145 mg, about 90 mg to about 140 mg, about 90 mg to about 135 mg, about 90 mg to about 130 mg, about 90 mg to about 125 mg, about 90 mg to about 120 mg, about 90 mg to about 115 mg, about 90 mg to about 110 mg, about 90 mg to about 105 mg, about 90 mg to about 100 mg, about 95 mg to about 150 mg, about 95 mg to about 145 mg, about 95 mg to about 140 mg, about 95 mg to about 135 mg, about 95 mg to about 130 mg, about 95 mg to about 125 mg, about 95 mg to about 120 mg, about 95 mg to about 115 mg, about 95 mg to about 110 mg, about 95 mg to about 105 mg, about 100 mg to about 150 mg, about 100 mg to about 145 mg, about 100 mg to about 140 mg, about 100 mg to about 135 mg, about 100 mg to about 130 mg, about 100 mg to about 125 mg, about 100 mg to about 120 mg, about 100 mg to about 115 mg, about 100 mg to about 110 mg, about 105 mg to about 150 mg, about 105 mg to about 145 mg, about 105 mg to about 140 mg, about 105 mg to about 135 mg, about 105 mg to about 130 mg, about 105 mg to about 125 mg, about 105 mg to about 120 mg, about 105 mg to about 115 mg, about 110 mg to about 150 mg, about 110 mg to about 145 mg, about 110 mg to about 140 mg, about 110 mg to about 135 mg, about 110 mg to about 130 mg, about 110 mg to about 125 mg, about 110 mg to about 120 mg, about 115 mg to about 150 mg, about 115 mg to about 145 mg, about 115 mg to about 140 mg, about 115 mg to about 135 mg, about 115 mg to about 130 mg, about 115 mg to about 125 mg, about 120 mg to about 150 mg, about 120 mg to about 145 mg, about 120 mg to about 140 mg, about 120 mg to about 135 mg, about 120 mg to about 130 mg, about 125 mg to about 150 mg, about 125 mg to about 145 mg, about 125 mg to about 140 mg, about 125 mg to about 135 mg, about 130 mg to about 150 mg, about 130 mg to about 145 mg, about 130 mg to about 140 mg, about 135 mg to about 150 mg, about 135 mg to about 145 mg, or about 140 mg to about 150 mg). In some embodiments, the time between administration of any two consecutive doses of the two or more doses is about 21 days.

In some embodiments, each of the two or more doses is about 80 mg to about 150 mg (e.g., about 80 mg to about 145 mg, about 80 mg to about 140 mg, about 80 mg to about 135 mg, about 80 mg to about 130 mg, about 80 mg to about 125 mg, about 80 mg to about 120 mg, about 80 mg to about 115 mg, about 80 mg to about 110 mg, about 80 mg to about 105 mg, about 80 mg to about 100 mg, about 80 mg to about 95 mg, about 80 mg to about 90 mg, about 85 mg to about 150 mg, about 85 mg to about 145 mg, about 85 mg to about 140 mg, about 85 mg to about 135 mg, about 85 mg to about 130 mg, about 85 mg to about 125 mg, about 85 mg to about 120 mg, about 85 mg to about 115 mg, about 85 mg to about 110 mg, about 85 mg to about 105 mg, about 85 mg to about 100 mg, about 85 mg to about 95 mg, about 90 mg to about 150 mg, about 90 mg to about 145 mg, about 90 mg to about 140 mg, about 90 mg to about 135 mg, about 90 mg to about 130 mg, about 90 mg to about 125 mg, about 90 mg to about 120 mg, about 90 mg to about 115 mg, about 90 mg to about 110 mg, about 90 mg to about 105 mg, about 90 mg to about 100 mg, about 95 mg to about 150 mg, about 95 mg to about 145 mg, about 95 mg to about 140 mg, about 95 mg to about 135 mg, about 95 mg to about 130 mg, about 95 mg to about 125 mg, about 95 mg to about 120 mg, about 95 mg to about 115 mg, about 95 mg to about 110 mg, about 95 mg to about 105 mg, about 100 mg to about 150 mg, about 100 mg to about 145 mg, about 100 mg to about 140 mg, about 100 mg to about 135 mg, about 100 mg to about 130 mg, about 100 mg to about 125 mg, about 100 mg to about 120 mg, about 100 mg to about 115 mg, about 100 mg to about 110 mg, about 105 mg to about 150 mg, about 105 mg to about 145 mg, about 105 mg to about 140 mg, about 105 mg to about 135 mg, about 105 mg to about 130 mg, about 105 mg to about 125 mg, about 105 mg to about 120 mg, about 105 mg to about 115 mg, about 110 mg to about 150 mg, about 110 mg to about 145 mg, about 110 mg to about 140 mg, about 110 mg to about 135 mg, about 110 mg to about 130 mg, about 110 mg to about 125 mg, about 110 mg to about 120 mg, about 115 mg to about 150 mg, about 115 mg to about 145 mg, about 115 mg to about 140 mg, about 115 mg to about 135 mg, about 115 mg to about 130 mg, about 115 mg to about 125 mg, about 120 mg to about 150 mg, about 120 mg to about 145 mg, about 120 mg to about 140 mg, about 120 mg to about 135 mg, about 120 mg to about 130 mg, about 125 mg to about 150 mg, about 125 mg to about 145 mg, about 125 mg to about 140 mg, about 125 mg to about 135 mg, about 130 mg to about 150 mg, about 130 mg to about 145 mg, about 130 mg to about 140 mg, about 135 mg to about 150 mg, about 135 mg to about 145 mg, or about 140 mg to about 150 mg). In some embodiments, the time between administration of any two consecutive doses of the two or more doses is about 21 days.

In some embodiments, each of the two or more doses is about 100 mg to about 150 mg (e.g., about 100 mg to about 145 mg, about 100 mg to about 140 mg, about 100 mg to about 135 mg, about 100 mg to about 130 mg, about 100 mg to about 125 mg, about 100 mg to about 120 mg, about 100 mg to about 115 mg, about 100 mg to about 110 mg, about 105 mg to about 150 mg, about 105 mg to about 145 mg, about 105 mg to about 140 mg, about 105 mg to about 135 mg, about 105 mg to about 130 mg, about 105 mg to about 125 mg, about 105 mg to about 120 mg, about 105 mg to about 115 mg, about 110 mg to about 150 mg, about 110 mg to about 145 mg, about 110 mg to about 140 mg, about 110 mg to about 135 mg, about 110 mg to about 130 mg, about 110 mg to about 125 mg, about 110 mg to about 120 mg, about 115 mg to about 150 mg, about 115 mg to about 145 mg, about 115 mg to about 140 mg, about 115 mg to about 135 mg, about 115 mg to about 130 mg, about 115 mg to about 125 mg, about 120 mg to about 150 mg, about 120 mg to about 145 mg, about 120 mg to about 140 mg, about 120 mg to about 135 mg, about 120 mg to about 130 mg, about 125 mg to about 150 mg, about 125 mg to about 145 mg, about 125 mg to about 140 mg, about 125 mg to about 135 mg, about 130 mg to about 150 mg, about 130 mg to about 145 mg, about 130 mg to about 140 mg, about 135 mg to about 150 mg, about 135 mg to about 145 mg, or about 140 mg to about 150 mg). In some embodiments, the time between administration of any two consecutive doses of the two or more doses is about 21 days.

In some embodiments, the two or more doses is at least three doses, at least four doses, at least five doses, at least six doses, at least seven doses, at least eight doses, at least nine doses, or at least 10 doses. In some embodiments, the two or more doses is 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 doses.

In some embodiments, the two or more doses are formulated for intramuscular administration, subcutaneous administration, intravenous administration, intrahepatic administration, or intraperitoneal administration. In some embodiments, the two or more doses are administered to the subject using intramuscular administration, subcutaneous administration, intravenous administration, intrahepatic administration, or intraperitoneal administration.

In some embodiments, the two or more doses are formulated for subcutaneous administration. In some embodiments, the two or more doses are administered to the subject using subcutaneous administration.

In some embodiments, the two or more doses are formulated for intravenous administration. In some embodiments, the two or more doses are administered to the subject using intravenous administration.

In some embodiments, the unresectable, advanced, recurrent, and/or metastatic solid tumor or the unresectable, advanced, recurrent, and/or metastatic solid cancer is selected from the group consisting of: bladder cancer, gastric cancer, breast cancer, cervical cancer, colorectal cancer, endometrial cancer, kidney cancer, lip cancer, liver cancer, melanoma, rhabdomyosarcoma, Ewing sarcoma, mesothelioma, lung cancer, non-small cell lung cancer, non-melanoma skin cancer, oral cancer, ovarian cancer, pancreatic cancer, sarcoma, osteosarcoma, glioblastoma, neuroblastoma, small cell lung cancer, thyroid cancer, retinoblastoma, urothelial carcinoma, renal cell carcinoma, esophageal cancer, prostate cancer, squamous cell head and neck carcinoma, hepatocellular cancer, testicular cancer, penile cancer, uterine cancer, vaginal cancer, gallbladder cancer, Merkel cell carcinoma, gastroesophageal junction cancer, microsatellite instability-high (MSI-H) solid tumor, a mismatch repair deficient (dMMR) solid tumor, and tumor mutational burden-high (TMB-H) solid tumor.

Methods for accessing successful treatment of unresectable, advanced, recurrent, and/or metastatic solid tumors and unresectable, advanced, recurrent, and/or metastatic solid cancers are known in the art.

In some embodiments, the method results in the achievement of stable disease (stable unresectable, advanced, recurrent, and/or metastatic solid tumor or stable unresectable, advanced, recurrent, and/or metastatic solid cancer) in the subject.

In some embodiments, the method results in a decrease (e.g., about a 1% decrease to about a 100% decrease, about a 1% decrease to about a 95% decrease, about a 1% decrease to about a 90% decrease, about a 1% decrease to about a 85% decrease, about a 1% decrease to about a 80% decrease, about a 1% decrease to about a 75% decrease, about a 1% decrease to about a 70% decrease, about a 1% decrease to about a 65% decrease, about a 1% decrease to about a 60% decrease, about a 1% decrease to about a 55% decrease, about a 1% decrease to about a 50% decrease, about a 1% decrease to about a 45% decrease, about a 1% decrease to about a 40% decrease, about a 1% decrease to about a 35% decrease, about a 1% decrease to about a 30% decrease, about a 1% decrease to about a 25% decrease, about a 1% decrease to about a 20% decrease, about a 1% decrease to about a 15% decrease, about a 1% decrease to about a 10% decrease, about a 1% decrease to about a 5% decrease, about a 5% decrease to about a 100% decrease, about a 5% decrease to about a 95% decrease, about a 5% decrease to about a 90% decrease, about a 5% decrease to about a 85% decrease, about a 5% decrease to about a 80% decrease, about a 5% decrease to about a 75% decrease, about a 5% decrease to about a 70% decrease, about a 5% decrease to about a 65% decrease, about a 5% decrease to about a 60% decrease, about a 5% decrease to about a 55% decrease, about a 5% decrease to about a 50% decrease, about a 5% decrease to about a 45% decrease, about a 5% decrease to about a 40% decrease, about a 5% decrease to about a 35% decrease, about a 5% decrease to about a 30% decrease, about a 5% decrease to about a 25% decrease, about a 5% decrease to about a 20% decrease, about a 5% decrease to about a 15% decrease, about a 5% decrease to about a 10% decrease, about a 10% decrease to about a 100% decrease, about a 10% decrease to about a 95% decrease, about a 10% decrease to about a 90% decrease, about a 10% decrease to about a 85% decrease, about a 10% decrease to about a 80% decrease, about a 10% decrease to about a 75% decrease, about a 10% decrease to about a 70% decrease, about a 10% decrease to about a 65% decrease, about a 10% decrease to about a 60% decrease, about a 10% decrease to about a 55% decrease, about a 10% decrease to about a 50% decrease, about a 10% decrease to about a 45% decrease, about a 10% decrease to about a 40% decrease, about a 10% decrease to about a 35% decrease, about a 10% decrease to about a 30% decrease, about a 10% decrease to about a 25% decrease, about a 10% decrease to about a 20% decrease, about a 10% decrease to about a 15% decrease, about a 15% decrease to about a 100% decrease, about a 15% decrease to about a 95% decrease, about a 15% decrease to about a 90% decrease, about a 15% decrease to about a 85% decrease, about a 15% decrease to about a 80% decrease, about a 15% decrease to about a 75% decrease, about a 15% decrease to about a 70% decrease, about a 15% decrease to about a 65% decrease, about a 15% decrease to about a 60% decrease, about a 15% decrease to about a 55% decrease, about a 15% decrease to about a 50% decrease, about a 15% decrease to about a 45% decrease, about a 15% decrease to about a 40% decrease, about a 15% decrease to about a 35% decrease, about a 15% decrease to about a 30% decrease, about a 15% decrease to about a 25% decrease, about a 15% decrease to about a 20% decrease, about a 20% decrease to about a 100% decrease, about a 20% decrease to about a 95% decrease, about a 20% decrease to about a 90% decrease, about a 20% decrease to about a 85% decrease, about a 20% decrease to about a 80% decrease, about a 20% decrease to about a 75% decrease, about a 20% decrease to about a 70% decrease, about a 20% decrease to about a 65% decrease, about a 20% decrease to about a 60% decrease, about a 20% decrease to about a 55% decrease, about a 20% decrease to about a 50% decrease, about a 20% decrease to about a 45% decrease, about a 20% decrease to about a 40% decrease, about a 20% decrease to about a 35% decrease, about a 20% decrease to about a 30% decrease, about a 20% decrease to about a 25% decrease, about a 25% decrease to about a 100% decrease, about a 25% decrease to about a 95% decrease, about a 25% decrease to about a 90% decrease, about a 25% decrease to about a 85% decrease, about a 25% decrease to about a 80% decrease, about a 25% decrease to about a 75% decrease, about a 25% decrease to about a 70% decrease, about a 25% decrease to about a 65% decrease, about a 25% decrease to about a 60% decrease, about a 25% decrease to about a 55% decrease, about a 25% decrease to about a 50% decrease, about a 25% decrease to about a 45% decrease, about a 25% decrease to about a 40% decrease, about a 25% decrease to about a 35% decrease, about a 25% decrease to about a 30% decrease, about a 30% decrease to about a 100% decrease, about a 30% decrease to about a 95% decrease, about a 30% decrease to about a 90% decrease, about a 30% decrease to about a 85% decrease, about a 30% decrease to about a 80% decrease, about a 30% decrease to about a 75% decrease, about a 30% decrease to about a 70% decrease, about a 30% decrease to about a 65% decrease, about a 30% decrease to about a 60% decrease, about a 30% decrease to about a 55% decrease, about a 30% decrease to about a 50% decrease, about a 30% decrease to about a 45% decrease, about a 30% decrease to about a 40% decrease, about a 30% decrease to about a 35% decrease, about a 35% decrease to about a 100% decrease, about a 35% decrease to about a 95% decrease, about a 35% decrease to about a 90% decrease, about a 35% decrease to about a 85% decrease, about a 35% decrease to about a 80% decrease, about a 35% decrease to about a 75% decrease, about a 35% decrease to about a 70% decrease, about a 35% decrease to about a 65% decrease, about a 35% decrease to about a 60% decrease, about a 35% decrease to about a 55% decrease, about a 35% decrease to about a 50% decrease, about a 35% decrease to about a 45% decrease, about a 35% decrease to about a 40% decrease, about a 40% decrease to about a 100% decrease, about a 40% decrease to about a 95% decrease, about a 40% decrease to about a 90% decrease, about a 40% decrease to about a 85% decrease, about a 40% decrease to about a 80% decrease, about a 40% decrease to about a 75% decrease, about a 40% decrease to about a 70% decrease, about a 40% decrease to about a 65% decrease, about a 40% decrease to about a 60% decrease, about a 40% decrease to about a 55% decrease, about a 40% decrease to about a 50% decrease, about a 40% decrease to about a 45% decrease, about a 45% decrease to about a 100% decrease, about a 45% decrease to about a 95% decrease, about a 45% decrease to about a 90% decrease, about a 45% decrease to about a 85% decrease, about a 45% decrease to about a 80% decrease, about a 45% decrease to about a 75% decrease, about a 45% decrease to about a 70% decrease, about a 45% decrease to about a 65% decrease, about a 45% decrease to about a 60% decrease, about a 45% decrease to about a 55% decrease, about a 45% decrease to about a 50% decrease, about a 50% decrease to about a 100% decrease, about a 50% decrease to about a 95% decrease, about a 50% decrease to about a 90% decrease, about a 50% decrease to about a 85% decrease, about a 50% decrease to about a 80% decrease, about a 50% decrease to about a 75% decrease, about a 50% decrease to about a 70% decrease, about a 50% decrease to about a 65% decrease, about a 50% decrease to about a 60% decrease, about a 50% decrease to about a 55% decrease, about a 55% decrease to about a 100% decrease, about a 55% decrease to about a 95% decrease, about a 55% decrease to about a 90% decrease, about a 55% decrease to about a 85% decrease, about a 55% decrease to about a 80% decrease, about a 55% decrease to about a 75% decrease, about a 55% decrease to about a 70% decrease, about a 55% decrease to about a 65% decrease, about a 55% decrease to about a 60% decrease, about a 60% decrease to about a 100% decrease, about a 60% decrease to about a 95% decrease, about a 60% decrease to about a 90% decrease, about a 60% decrease to about a 85% decrease, about a 60% decrease to about a 80% decrease, about a 60% decrease to about a 75% decrease, about a 60% decrease to about a 70% decrease, about a 60% decrease to about a 65% decrease, about a 65% decrease to about a 100% decrease, about a 65% decrease to about a 95% decrease, about a 65% decrease to about a 90% decrease, about a 65% decrease to about a 85% decrease, about a 65% decrease to about a 80% decrease, about a 65% decrease to about a 75% decrease, about a 65% decrease to about a 70% decrease, about a 70% decrease to about a 100% decrease, about a 70% decrease to about a 95% decrease, about a 70% decrease to about a 90% decrease, about a 70% decrease to about a 85% decrease, about a 70% decrease to about a 80% decrease, about a 70% decrease to about a 75% decrease, about a 75% decrease to about a 100% decrease, about a 75% decrease to about a 95% decrease, about a 75% decrease to about a 90% decrease, about a 75% decrease to about a 85% decrease, about a 75% decrease to about a 80% decrease, about a 80% decrease to about a 100% decrease, about a 80% decrease to about a 95% decrease, about a 80% decrease to about a 90% decrease, about a 80% decrease to about a 85% decrease, about a 85% decrease to about a 100% decrease, about a 85% decrease to about a 95% decrease, about a 85% decrease to about a 90% decrease, about a 90% decrease to about a 100% decrease, about a 90% decrease to about a 95% decrease, about a 95% decrease to about a 100% decrease, or about a 95% to about a 99% decrease) in the rate of progression of the unresectable, advanced, recurrent, and/or metastatic solid tumor or unresectable, advanced, recurrent, and/or metastatic solid cancer, e.g., as compared to the rate of progression before treatment or the rate of progression in a similar subject identified as having a similar unresectable, advanced, recurrent, and/or metastatic solid tumor or similar unresectable, advanced, recurrent, and/or metastatic solid cancer and receiving no treatment or a different treatment.

In some embodiments, the method results in a decrease (e.g., about a 1% decrease to about a 100% decrease, about a 1% decrease to about a 95% decrease, about a 1% decrease to about a 90% decrease, about a 1% decrease to about a 85% decrease, about a 1% decrease to about a 80% decrease, about a 1% decrease to about a 75% decrease, about a 1% decrease to about a 70% decrease, about a 1% decrease to about a 65% decrease, about a 1% decrease to about a 60% decrease, about a 1% decrease to about a 55% decrease, about a 1% decrease to about a 50% decrease, about a 1% decrease to about a 45% decrease, about a 1% decrease to about a 40% decrease, about a 1% decrease to about a 35% decrease, about a 1% decrease to about a 30% decrease, about a 1% decrease to about a 25% decrease, about a 1% decrease to about a 20% decrease, about a 1% decrease to about a 15% decrease, about a 1% decrease to about a 10% decrease, about a 1% decrease to about a 5% decrease, about a 5% decrease to about a 100% decrease, about a 5% decrease to about a 95% decrease, about a 5% decrease to about a 90% decrease, about a 5% decrease to about a 85% decrease, about a 5% decrease to about a 80% decrease, about a 5% decrease to about a 75% decrease, about a 5% decrease to about a 70% decrease, about a 5% decrease to about a 65% decrease, about a 5% decrease to about a 60% decrease, about a 5% decrease to about a 55% decrease, about a 5% decrease to about a 50% decrease, about a 5% decrease to about a 45% decrease, about a 5% decrease to about a 40% decrease, about a 5% decrease to about a 35% decrease, about a 5% decrease to about a 30% decrease, about a 5% decrease to about a 25% decrease, about a 5% decrease to about a 20% decrease, about a 5% decrease to about a 15% decrease, about a 5% decrease to about a 10% decrease, about a 10% decrease to about a 100% decrease, about a 10% decrease to about a 95% decrease, about a 10% decrease to about a 90% decrease, about a 10% decrease to about a 85% decrease, about a 10% decrease to about a 80% decrease, about a 10% decrease to about a 75% decrease, about a 10% decrease to about a 70% decrease, about a 10% decrease to about a 65% decrease, about a 10% decrease to about a 60% decrease, about a 10% decrease to about a 55% decrease, about a 10% decrease to about a 50% decrease, about a 10% decrease to about a 45% decrease, about a 10% decrease to about a 40% decrease, about a 10% decrease to about a 35% decrease, about a 10% decrease to about a 30% decrease, about a 10% decrease to about a 25% decrease, about a 10% decrease to about a 20% decrease, about a 10% decrease to about a 15% decrease, about a 15% decrease to about a 100% decrease, about a 15% decrease to about a 95% decrease, about a 15% decrease to about a 90% decrease, about a 15% decrease to about a 85% decrease, about a 15% decrease to about a 80% decrease, about a 15% decrease to about a 75% decrease, about a 15% decrease to about a 70% decrease, about a 15% decrease to about a 65% decrease, about a 15% decrease to about a 60% decrease, about a 15% decrease to about a 55% decrease, about a 15% decrease to about a 50% decrease, about a 15% decrease to about a 45% decrease, about a 15% decrease to about a 40% decrease, about a 15% decrease to about a 35% decrease, about a 15% decrease to about a 30% decrease, about a 15% decrease to about a 25% decrease, about a 15% decrease to about a 20% decrease, about a 20% decrease to about a 100% decrease, about a 20% decrease to about a 95% decrease, about a 20% decrease to about a 90% decrease, about a 20% decrease to about a 85% decrease, about a 20% decrease to about a 80% decrease, about a 20% decrease to about a 75% decrease, about a 20% decrease to about a 70% decrease, about a 20% decrease to about a 65% decrease, about a 20% decrease to about a 60% decrease, about a 20% decrease to about a 55% decrease, about a 20% decrease to about a 50% decrease, about a 20% decrease to about a 45% decrease, about a 20% decrease to about a 40% decrease, about a 20% decrease to about a 35% decrease, about a 20% decrease to about a 30% decrease, about a 20% decrease to about a 25% decrease, about a 25% decrease to about a 100% decrease, about a 25% decrease to about a 95% decrease, about a 25% decrease to about a 90% decrease, about a 25% decrease to about a 85% decrease, about a 25% decrease to about a 80% decrease, about a 25% decrease to about a 75% decrease, about a 25% decrease to about a 70% decrease, about a 25% decrease to about a 65% decrease, about a 25% decrease to about a 60% decrease, about a 25% decrease to about a 55% decrease, about a 25% decrease to about a 50% decrease, about a 25% decrease to about a 45% decrease, about a 25% decrease to about a 40% decrease, about a 25% decrease to about a 35% decrease, about a 25% decrease to about a 30% decrease, about a 30% decrease to about a 100% decrease, about a 30% decrease to about a 95% decrease, about a 30% decrease to about a 90% decrease, about a 30% decrease to about a 85% decrease, about a 30% decrease to about a 80% decrease, about a 30% decrease to about a 75% decrease, about a 30% decrease to about a 70% decrease, about a 30% decrease to about a 65% decrease, about a 30% decrease to about a 60% decrease, about a 30% decrease to about a 55% decrease, about a 30% decrease to about a 50% decrease, about a 30% decrease to about a 45% decrease, about a 30% decrease to about a 40% decrease, about a 30% decrease to about a 35% decrease, about a 35% decrease to about a 100% decrease, about a 35% decrease to about a 95% decrease, about a 35% decrease to about a 90% decrease, about a 35% decrease to about a 85% decrease, about a 35% decrease to about a 80% decrease, about a 35% decrease to about a 75% decrease, about a 35% decrease to about a 70% decrease, about a 35% decrease to about a 65% decrease, about a 35% decrease to about a 60% decrease, about a 35% decrease to about a 55% decrease, about a 35% decrease to about a 50% decrease, about a 35% decrease to about a 45% decrease, about a 35% decrease to about a 40% decrease, about a 40% decrease to about a 100% decrease, about a 40% decrease to about a 95% decrease, about a 40% decrease to about a 90% decrease, about a 40% decrease to about a 85% decrease, about a 40% decrease to about a 80% decrease, about a 40% decrease to about a 75% decrease, about a 40% decrease to about a 70% decrease, about a 40% decrease to about a 65% decrease, about a 40% decrease to about a 60% decrease, about a 40% decrease to about a 55% decrease, about a 40% decrease to about a 50% decrease, about a 40% decrease to about a 45% decrease, about a 45% decrease to about a 100% decrease, about a 45% decrease to about a 95% decrease, about a 45% decrease to about a 90% decrease, about a 45% decrease to about a 85% decrease, about a 45% decrease to about a 80% decrease, about a 45% decrease to about a 75% decrease, about a 45% decrease to about a 70% decrease, about a 45% decrease to about a 65% decrease, about a 45% decrease to about a 60% decrease, about a 45% decrease to about a 55% decrease, about a 45% decrease to about a 50% decrease, about a 50% decrease to about a 100% decrease, about a 50% decrease to about a 95% decrease, about a 50% decrease to about a 90% decrease, about a 50% decrease to about a 85% decrease, about a 50% decrease to about a 80% decrease, about a 50% decrease to about a 75% decrease, about a 50% decrease to about a 70% decrease, about a 50% decrease to about a 65% decrease, about a 50% decrease to about a 60% decrease, about a 50% decrease to about a 55% decrease, about a 55% decrease to about a 100% decrease, about a 55% decrease to about a 95% decrease, about a 55% decrease to about a 90% decrease, about a 55% decrease to about a 85% decrease, about a 55% decrease to about a 80% decrease, about a 55% decrease to about a 75% decrease, about a 55% decrease to about a 70% decrease, about a 55% decrease to about a 65% decrease, about a 55% decrease to about a 60% decrease, about a 60% decrease to about a 100% decrease, about a 60% decrease to about a 95% decrease, about a 60% decrease to about a 90% decrease, about a 60% decrease to about a 85% decrease, about a 60% decrease to about a 80% decrease, about a 60% decrease to about a 75% decrease, about a 60% decrease to about a 70% decrease, about a 60% decrease to about a 65% decrease, about a 65% decrease to about a 100% decrease, about a 65% decrease to about a 95% decrease, about a 65% decrease to about a 90% decrease, about a 65% decrease to about a 85% decrease, about a 65% decrease to about a 80% decrease, about a 65% decrease to about a 75% decrease, about a 65% decrease to about a 70% decrease, about a 70% decrease to about a 100% decrease, about a 70% decrease to about a 95% decrease, about a 70% decrease to about a 90% decrease, about a 70% decrease to about a 85% decrease, about a 70% decrease to about a 80% decrease, about a 70% decrease to about a 75% decrease, about a 75% decrease to about a 100% decrease, about a 75% decrease to about a 95% decrease, about a 75% decrease to about a 90% decrease, about a 75% decrease to about a 85% decrease, about a 75% decrease to about a 80% decrease, about a 80% decrease to about a 100% decrease, about a 80% decrease to about a 95% decrease, about a 80% decrease to about a 90% decrease, about a 80% decrease to about a 85% decrease, about a 85% decrease to about a 100% decrease, about a 85% decrease to about a 95% decrease, about a 85% decrease to about a 90% decrease, about a 90% decrease to about a 100% decrease, about a 90% decrease to about a 95% decrease, about a 95% decrease to about a 100% decrease, or about a 95% to about a 99% decrease) in the size or volume of the unresectable, advanced, recurrent, and/or metastatic solid tumor, e.g., e.g., as compared to the size or volume of the unresectable, advanced, recurrent, and/or metastatic solid tumor before treatment.

### Additional Therapeutic Agents

Some embodiments of any of the methods described herein can further include administering to a subject (e.g., any of the subjects described herein) a therapeutically effective amount of one or more additional therapeutic agents. The one or more additional therapeutic agents can be administered to the subject at substantially the same time as the multi-chain chimeric polypeptide (e.g., any of the multi-chain chimeric polypeptides described herein). In some embodiments, one or more additional therapeutic agents can be administered to the subject prior to administration of the multi-chain chimeric polypeptide (e.g., any of the multi-chain chimeric polypeptides described herein). In some embodiments, one or more additional therapeutic agents can be administered to the subject after administration of the multi-chain chimeric polypeptide (e.g., any of the multi-chain chimeric polypeptides described herein) to the subject.

Non-limiting examples of additional therapeutic agents include: anti-inflammatory agents, anti-cancer drugs, activating receptor agonists, immune checkpoint inhibitors, agents for blocking HLA-specific inhibitory receptors, Glucogen Synthase Kinase (GSK) 3 inhibitors, antibodies, and ex-vivo activated immune cells (e.g., CAR-T cells or CAR-NK cells).

Non-limiting examples of anticancer drugs include antimetabolic drugs (e.g., 5-fluorouracil (5-FU), 6-mercaptopurine (6-MP), capecitabine, cytarabine, floxuridine, fludarabine, gemcitabine, hydroxycarbamide, methotrexate, 6-thioguanine, cladribine, nelarabine, pentostatin, or pemetrexed), plant alkaloids (e.g., vinblastine, vincristine, vindesine, camptothecin, 9-methoxycamptothecin, coronaridine, taxol, naucleaorals, diprenylated indole alkaloid, montamine, schischkiniin, protoberberine, berberine, sanguinarine, chelerythrine, chelidonine, liriodenine, clivorine, β-carboline, antofine, tylophorine, cryptolepine, neocryptolepine, corynoline, sampangine, carbazole, crinamine, montanine, ellipticine, paclitaxel, docetaxel, etoposide, tenisopide, irinotecan, topotecan, or acridone alkaloids), proteasome inhibitors (e.g., lactacystin, disulfiram, epigallocatechin-3-gallate, marizomib (salinosporamide A), oprozomib (ONX-0912), delanzomib (CEP-18770), epoxomicin, MG132, beta-hydroxy beta-methylbutyrate, bortezomib, carfilzomib, or ixazomib), antitumor antibiotics (e.g., doxorubicin, daunorubicin, epirubicin, mitoxantrone, idarubicin, actinomycin, plicamycin, mitomycin, or bleomycin), histone deacetylase inhibitors (e.g., vorinostat, panobinostat, belinostat, givinostat, abexinostat, depsipeptide, entinostat, phenyl butyrate, valproic acid, trichostatin A, dacinostat, mocetinostat, pracinostat, nicotinamide, cambinol, tenovin 1, tenovin 6, sirtinol, ricolinostat, tefinostat, kevetrin, quisinostat, resminostat, tacedinaline, chidamide, or selisistat), tyrosine kinase inhibitors (e.g., axitinib, dasatinib, encorafinib, erlotinib, imatinib, nilotinib, pazopanib, and sunitinib), and chemotherapeutic agents (e.g., all-trans retinoic acid, azacitidine, azathioprine, doxifluridine, epothilone, hydroxyurea, imatinib, teniposide, tioguanine, valrubicin, vemurafenib, and lenalidomide). Additional examples of chemotherapeutic agents include alkylating agents, e.g., mechlorethamine, cyclophosphamide, chlorambucil, melphalan, ifosfamide, thiotepa, hexamethylmelamine, busulfan, altretamine, procarbazine, dacarbazine, temozolomide, carmustine, lumustine, streptozocin, carboplatin, cisplatin, and oxaliplatin.

Non-limiting examples of activating receptor agonists include any agonists for activating receptors which activate and enhance the cytotoxicity of NK cells, including anti-CD16 antibodies (e.g., anti-CD16/CD30 bispecific monoclonal antibody (BiMAb)) and Fc-based fusion proteins. Non-limiting examples of checkpoint inhibitors include anti-PD-1 antibodies (e.g., MEDI0680), anti-PD-L1 antibodies (e.g., BCD-135, BGB-A333, CBT-502, CK-301, CS1001, FAZ053, KN035, MDX-1105, MSB2311, SHR-1316, anti-PD-L1/CTLA-4 bispecific antibody KN046, anti-PD-L1/TGFβRII fusion protein M7824, anti-PD-L1/TIM-3 bispecific antibody LY3415244, atezolizumab, or avelumab), anti-TIM3 antibodies (e.g., TSR-022, Sym023, or MBG453) and anti-CTLA-4 antibodies (e.g., AGEN1884, MK-1308, or an anti-CTLA-4/OX40 bispecific antibody ATOR-1015). Non-limiting examples of agents for blocking HLA-specific inhibitory receptors include monalizumab (e.g., an anti-HLA-E NKG2A inhibitory receptor monoclonal antibody). Non-limiting examples of GSK3 inhibitor include tideglusib or CHIR99021. Non-limiting examples of antibodies that can be used as additional therapeutic agents include anti-CD26 antibodies (e.g., YS110), anti-CD36 antibodies, and any other antibody or antibody construct that can bind to and activate an Fc receptor (e.g., CD16) on a NK cell. In some embodiments, an additional therapeutic agent can be insulin or metformin.

Non-limiting examples of in-vitro activated immune cells include regulatory T cells, CAR-regulatory T cells, NK cells, CAR-NK cells, cytotoxic T cells, and CAR-cytotoxic T cells.

### EXAMPLES

The invention is further described in the following examples, which do not limit the scope of the invention described in the claims.

### Example 1. Construction of Exemplary Multi-Chain Chimeric Polypeptides and Evaluation of Properties Thereof

Two multi-chain chimeric polypeptides were generated and their properties were evaluated. Each of the two multi-chain chimeric polypeptides includes a first chimeric polypeptide that includes a soluble tissue factor domain covalently linked a first target-binding domain and a first domain of an affinity pair of domains. The second chimeric polypeptide in each of the two multi-chain chimeric polypeptides includes a second domain of the affinity pair of domains, and a second target-binding domain. Activity data of various chimeric polypeptides is demonstrated for example in U.S. application Ser. Nos. 17/819,240 and 16/555,689 each of which are herein incorporated by reference in their entirety.

### Description of Logic Underlying Construction of Multi-Chain Chimeric Polypeptides

Tissue Factor (TF) is a stable, transmembrane protein containing 236 amino acid residues. The truncated, recombinant 219-amino-acid extracellular domain of tissue factor is soluble and is known to be expressed at high levels in bacteria or mammalian cells. Without wishing to be bound to a particular theory, the applicants speculated that the 219-aa tissue factor could be used as a connector linker for creation of unique multi-chain chimeric polypeptides.

First chimeric polypeptides including soluble tissue factor domain were produced at high levels by CHO cells grown in fermentation broth. These first chimeric polypeptides were purified by an anti-tissue factor monoclonal antibody (mAb) coupled on a solid matrix. Notably, tissue factor contains binding sites for FVIIa and FX. The catalytic activity of the tissue factor-FVIIa complex for FX is approximately 1 million-fold lower when tissue factor is not anchored to a phospholipid bilayer. Thus, without wishing to be bound to a particular theory, applicants speculated that using the 219-aa extracellular domain of tissue factor without the transmembrane in construction of the first chimeric polypeptides may eliminate the pro-coagulation activity of tissue factor in the first chimeric polypeptides. In an effort to further reduce or eliminate the pro-coagulation activity of the 219-aa tissue factor, select mutations in tissue factor can be made, specifically at seven amino acid residues that are known to contribute to binding energy of the FVIIa binding site.

### Characterization of Binding Interactions for Described Chimeric Polypeptides

To determine if the first and second chimeric polypeptides bind to each other to form multi-chain chimeric polypeptides, in vitro binding assays were performed. To determine if the first chimeric polypeptide comprising soluble tissue factor domain are recognized and bound by anti-TF mAb, in vitro binding assays were performed. Notably, the data indicated that the mutated tissue factor proteins are still recognized and selectively bound by the anti-TF mAb which is known to bind to the FX binding site on tissue factor. To determine if the first chimeric polypeptides comprising soluble tissue factor domain covalently linked to scFvs or cytokines (see FIG. 1 and FIG. 2) possess functional scFvs or cytokines, in vitro binding assays were performed. The data from the aforementioned assays were consistent with the purified first chimeric polypeptides having the expected biological activities (e.g. scFvs selectively bind expected target antigens or cytokines selectively bind expected receptors or binding proteins).

In addition, experiments performed using the two multi-chain chimeric polypeptides including a first and second chimeric polypeptide bound to each other demonstrate the expected target binding activity (e.g., the multi-chain chimeric polypeptide binds specifically to the target specifically recognized by the first target-binding domain and the target specifically recognized by the second target-binding domain).

Based on the aforementioned results, applicants concluded that the soluble tissue factor connecter linker provided or enabled appropriate display of the polypeptides encoding either scFvs, interleukins, cytokines, interleukin receptors, or cytokine receptors in three-dimensional space relative to soluble tissue factor domain and relative to one another such that each retained expected biological properties and activities.

When both the first and second chimeric polypeptides were co-expressed, the heterodimeric complexes were secreted into the fermentation broths at high levels. The complexes were captured and readily purified by anti-TF mAb conjugated to a solid matrix using affinity chromatography. The first and second target-binding domains of these multi-chain chimeric polypeptides retained their expected biological activities as assayed by in vitro binding assays. Thus, the assembly of the multi-chain chimeric polypeptides provides the appropriate spatial display and folding of the domains for biological activities. Importantly, the spatial arrangement of the multi-chain chimeric polypeptides does not interfere with the FX binding site on tissue factor which enables the use of anti-TF mAb for affinity purification.

### Characterization of Stability for Described Chimeric Polypeptides

Both purified multi-chain chimeric polypeptides are stable. These multi-chain chimeric polypeptides are structurally intact and fully biologically active when they are incubated in human serum at 37° C. for 72 hours.

### Characterization of Propensity of Described Chimeric Polypeptides to Aggregate

Both purified multi-chain chimeric polypeptides developed do not form aggregates when stored at 4° C. in PBS.

Characterization of viscosity of described chimeric polypeptides There is no viscosity issue when the multi-chain chimeric polypeptides are formulated at a concentration as high as 50 mg/mL in PBS.

### Additional Applications of the Multi-Chain Chimeric Polypeptide Platform

The data from these studies show that the platform technologies described herein can be utilized to create molecules that could be fused to target-binding domains derived from antibodies, in any of the formats as described herein including, without limitation, adhesion molecules, receptors, cytokines, ligands, and chemokines. With the appropriate target-binding domain, the resulting multi-chain chimeric polypeptides could promote conjugation of various immune effector cells and mediate destruction of target cells, including cancer cells, virally-infected cells, or senescent cells. Other domains in the multi-chain chimeric polypeptides stimulate, activate, and attract the immune system for enhancing cytotoxicity of effector cells for the targeted cells.

### Example 2: TGFRt15-TGFRs Fusion Protein Generation and Characterization

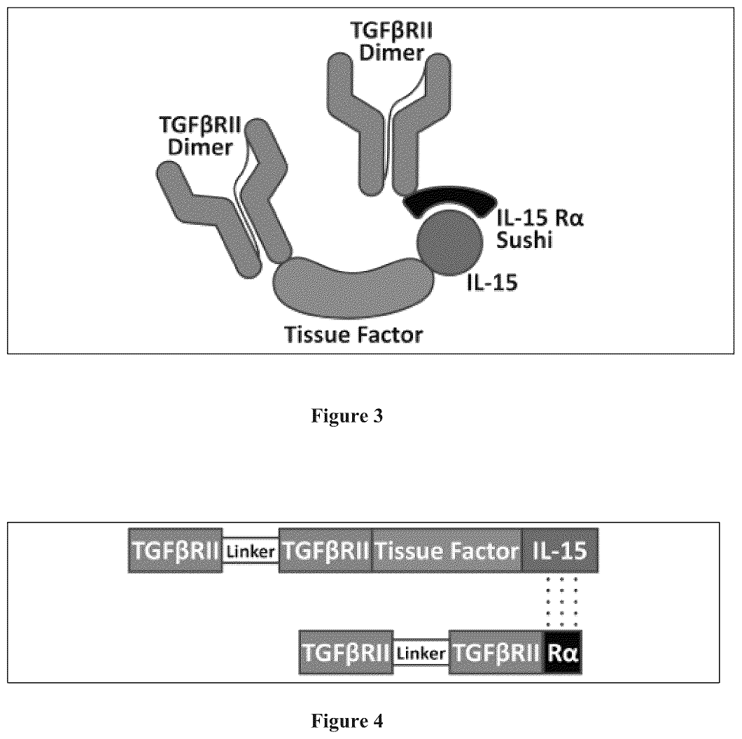

A fusion protein complex was generated comprising of TGFβ Receptor II/IL-15RαSu and TGFβ Receptor II/TF/IL-15 fusion proteins (FIG. 3 and FIG. 4). The human TGFβ Receptor II (Ile24-Asp159), tissue factor 219, and IL-15 sequences were obtained from the UniProt website and DNA for these sequences was synthesized by Genewiz. Specifically, a construct was made linking two TGFβ Receptor II sequences with a G4S(3) linker to generate a single chain version of TGFβ Receptor II and then directly linking to the N-terminus coding region of tissue factor 219 followed by the N-terminus coding region of IL-15.

The nucleic acid and protein sequences of a construct comprising two TGFβ Receptor II linked to the N-terminus of tissue factor 219 following with the N-terminus of IL-15 are shown below.

The nucleic acid sequence of the two TGFβ Receptor II/TF/IL-15 construct (including signal peptide sequence) is as follows (SEQ ID NO:27):

(Signal peptide)
ATGAAGTGGGTGACCTTCATCAGCCTGCTGTTCCTGTTCTCCAGCG
CCTACTCC
(Two Human TGFβ Receptor II fragments)
ATCCCCCCCCATGTGCAAAAGAGCGTGAACAACGATATGATCGTGA
CCGACAACAACGGCGCCGTGAAGTTTCCCCAGCTCTGCAAGTTCTG
CGATGTCAGGTTCAGCACCTGCGATAATCAGAAGTCCTGCATGTCC
AACTGCAGCATCACCTCCATCTGCGAGAAGCCCCAAGAAGTGTGCG
TGGCCGTGTGGCGGAAAAATGACGAGAACATCACCCTGGAGACCGT
GTGTCACGACCCCAAGCTCCCTTATCACGACTTCATTCTGGAGGAC
GCTGCCTCCCCCAAATGCATCATGAAGGAGAAGAAGAAGCCCGGAG
AGACCTTCTTTATGTGTTCCTGTAGCAGCGACGAGTGTAACGACAA
CATCATCTTCAGCGAAGAGTACAACACCAGCAACCCTGATGGAGGT
GGCGGATCCGGAGGTGGAGGTTCTGGTGGAGGTGGGAGTATTCCTC
CCCACGTGCAGAAGAGCGTGAATAATGACATGATCGTGACCGATAA
CAATGGCGCCGTGAAATTTCCCCAGCTGTGCAAATTCTGCGATGTG
AGGTTTTCCACCTGCGACAACCAGAAGTCCTGTATGAGCAACTGCT
CCATCACCTCCATCTGTGAGAAGCCTCAGGAGGTGTGCGTGGCTGT
CTGGCGGAAGAATGACGAGAATATCACCCTGGAAACCGTCTGCCAC
GATCCCAAGCTGCCCTACCACGATTTCATCCTGGAAGACGCCGCCA
GCCCTAAGTGCATCATGAAAGAGAAAAAGAAGCCTGGCGAGACCTT
TTTCATGTGCTCCTGCAGCAGCGACGAATGCAACGACAATATCATC
TTTAGCGAGGAATACAATACCAGCAACCCCGAC
(Human Tissue Factor 219)
AGCGGCACAACCAACACAGTCGCTGCCTATAACCTCACTTGGAAGA
GCACCAACTTCAAAACCATCCTCGAATGGGAACCCAAACCCGTTAA
CCAAGTTTACACCGTGCAGATCAGCACCAAGTCCGGCGACTGGAAG
TCCAAATGTTTCTATACCACCGACACCGAGTGCGATCTCACCGATG
AGATCGTGAAAGATGTGAAACAGACCTACCTCGCCCGGGTGTTTAG
CTACCCCGCCGGCAATGTGGAGAGCACTGGTTCCGCTGGCGAGCCT
TTATACGAGAACAGCCCCGAATTTACCCCTTACCTCGAGACCAATT
TAGGACAGCCCACCATCCAAAGCTTTGAGCAAGTTGGCACAAAGGT
GAATGTGACAGTGGAGGACGAGCGGACTTTAGTGCGGCGGAACAAC
ACCTTTCTCAGCCTCCGGGATGTGTTCGGCAAAGATTTAATCTACA
CACTGTATTACTGGAAGTCCTCTTCCTCCGGCAAGAAGACAGCTAA
AACCAACACAAACGAGTTTTTAATCGACGTGGATAAAGGCGAAAAC
TACTGTTTCAGCGTGCAAGCTGTGATCCCCTCCCGGACCGTGAATA
GGAAAAGCACCGATAGCCCCGTTGAGTGCATGGGCCAAGAAAAGGG
CGAGTTCCGGGAG
(Human IL-15)
AACTGGGTGAACGTCATCAGCGATTTAAAGAAGATCGAAGATTTAA
TTCAGTCCATGCATATCGACGCCACTTTATACACAGAATCCGACGT
GCACCCCTCTTGTAAGGTGACCGCCATGAAATGTTTTTTACTGGAG
CTGCAAGTTATCTCTTTAGAGAGCGGAGACGCTAGCATCCACGACA
CCGTGGAGAATTTAATCATTTTAGCCAATAACTCTTTATCCAGCAA
CGGCAACGTGACAGAGTCCGGCTGCAAGGAGTGCGAAGAGCTGGAG
GAGAAGAACATCAAGGAGTTTCTGCAATCCTTTGTGCACATTGTCC
AGATGTTCATCAATACCTCC

The amino acid sequence of TGFβ Receptor II/TF/IL-15 fusion protein (including the leader sequence) is as follows (SEQ ID NO:7):

(Signal peptide)
MKWVTFISLLFLFSSAYS
(Human TGFβ Receptor II)
IPPHVQKSVNNDMIVTDNNGAVKFPQLCKFCDVRFSTCDNQKSCMS
NCSITSICEKPQEVCVAVWRKNDENITLETVCHDPKLPYHDFILED
AASPKCIMKEKKKPGETFFMCSCSSDECNDNIIFSEEYNTSNPDGG
GGSGGGGSGGGGSIPPHVQKSVNNDMIVTDNNGAVKFPQLCKFCDV
RFSTCDNQKSCMSNCSITSICEKPQEVCVAVWRKNDENITLETVCH
DPKLPYHDFILEDAASPKCIMKEKKKPGETFFMCSCSSDECNDNII
FSEEYNTSNPD
(Human Tissue Factor 219)
SGTTNTVAAYNLTWKSTNFKTILEWEPKPVNQVYTVQISTKSGDWK
SKCFYTTDTECDLTDEIVKDVKQTYLARVFSYPAGNVESTGSAGEP
LYENSPEFTPYLETNLGQPTIQSFEQVGTKVNVTVEDERTLVRRNN
TFLSLRDVFGKDLIYTLYYWKSSSSGKKTAKTNTNEFLIDVDKGEN
YCFSVQAVIPSRTVNRKSTDSPVECMGQEKGEFRE
(Human IL-15)
NWVNVISDLKKIEDLIQSMHIDATLYTESDVHPSCKVTAMKCFLLE
LQVISLESGDASIHDTVENLIILANNSLSSNGNVTESGCKECEELE
EKNIKEFLQSFVHIVQMFINTS

Constructs were also made by attaching two TGFβ Receptor II directly to the IL-15RαSu chain which was synthesized by Genewiz. The nucleic acid and protein sequences of a construct comprising the TGFβ Receptor II linked to the N-terminus of IL-15RαSu are shown below.

The nucleic acid sequence of the TGFβ Receptor II/IL-15 RαSu construct (including signal peptide sequence) is as follows (SEQ ID NO:29):

(Signal peptide)
ATGAAGTGGGTGACCTTCATCAGCCTGCTGTTCCTGTTCTCCAGCG
CCTACTCC
(Two human TGFβ Receptor II fragments)
ATCCCCCCCCATGTGCAAAAGAGCGTGAACAACGATATGATCGTGA
CCGACAACAACGGCGCCGTGAAGTTTCCCCAGCTCTGCAAGTTCTG
CGATGTCAGGTTCAGCACCTGCGATAATCAGAAGTCCTGCATGTCC
AACTGCAGCATCACCTCCATCTGCGAGAAGCCCCAAGAAGTGTGCG
TGGCCGTGTGGCGGAAAAATGACGAGAACATCACCCTGGAGACCGT
GTGTCACGACCCCAAGCTCCCTTATCACGACTTCATTCTGGAGGAC
GCTGCCTCCCCCAAATGCATCATGAAGGAGAAGAAGAAGCCCGGAG
AGACCTTCTTTATGTGTTCCTGTAGCAGCGACGAGTGTAACGACAA
CATCATCTTCAGCGAAGAGTACAACACCAGCAACCCTGATGGAGGT
GGCGGATCCGGAGGTGGAGGTTCTGGTGGAGGTGGGAGTATTCCTC
CCCACGTGCAGAAGAGCGTGAATAATGACATGATCGTGACCGATAA
CAATGGCGCCGTGAAATTTCCCCAGCTGTGCAAATTCTGCGATGTG
AGGTTTTCCACCTGCGACAACCAGAAGTCCTGTATGAGCAACTGCT
CCATCACCTCCATCTGTGAGAAGCCTCAGGAGGTGTGCGTGGCTGT
CTGGCGGAAGAATGACGAGAATATCACCCTGGAAACCGTCTGCCAC
GATCCCAAGCTGCCCTACCACGATTTCATCCTGGAAGACGCCGCCA
GCCCTAAGTGCATCATGAAAGAGAAAAAGAAGCCTGGCGAGACCTT
TTTCATGTGCTCCTGCAGCAGCGACGAATGCAACGACAATATCATC
TTTAGCGAGGAATACAATACCAGCAACCCCGAC
(Human IL-15R α sushi domain)
ATTACATGCCCCCCTCCCATGAGCGTGGAGCACGCCGACATCTGGG
TGAAGAGCTATAGCCTCTACAGCCGGGAGAGGTATATCTGTAACAG
CGGCTTCAAGAGGAAGGCCGGCACCAGCAGCCTCACCGAGTGCGTG
CTGAATAAGGCTACCAACGTGGCTCACTGGACAACACCCTCTTTAA
AGTGCATCCGG

The amino acid sequence of the two TGFβ Receptor II/IL-15RαSu construct (including signal peptide sequence) is as follows (SEQ ID NO:8):

(Signal peptide)
MKWVTFISLLFLFSSAYS
(Two human TGFβ Receptor II extra-cellular
domains)
IPPHVQKSVNNDMIVTDNNGAVKFPQLCKFCDVRFSTCDNQKSCMS
NCSITSICEKPQEVCVAVWRKNDENITLETVCHDPKLPYHDFILED
AASPKCIMKEKKKPGETFFMCSCSSDECNDNIIFSEEYNTSNPDGG
GGSGGGGSGGGGSIPPHVQKSVNNDMIVTDNNGAVKFPQLCKFCDV
RFSTCDNQKSCMSNCSITSICEKPQEVCVAVWRKNDENITLETVCH
DPKLPYHDFILEDAASPKCIMKEKKKPGETFFMCSCSSDECNDNII
FSEEYNTSNPD
(Human IL-15R α sushi domain)
ITCPPPMSVEHADIWVKSYSLYSRERYICNSGFKRKAGTSSLTECV
LNKATNVAHWTTPSLKCIR

In some cases, the leader peptide is cleaved from the intact polypeptide to generate the mature form that may be soluble or secreted.

The TGFβR/IL-15RαSu and TGFβR/TF/IL-15 constructs were cloned into a modified retrovirus expression vectors as described previously (Hughes M S, Yu Y Y, Dudley M E, Zheng Z, Robbins P F, Li Y, et al. Transfer of a TCR gene derived from a patient with a marked antitumor response conveys highly active T-cell effector functions. Hum Gene Ther 2005; 16:457-72), and the expression vectors were transfected into CHO-K1 cells. Co-expression of the two constructs in CHO-K1 cells allowed for formation and secretion of the soluble TGFβR/TF/IL-15:TGFβR/IL-15RαSu protein complex (referred to as TGFRt15-TGFRs), which can be purified by anti-TF IgG1 affinity and other chromatography methods.

### Example 3. Protection by TGFRt15-TGFRs from Chemical Induced Liver Damage

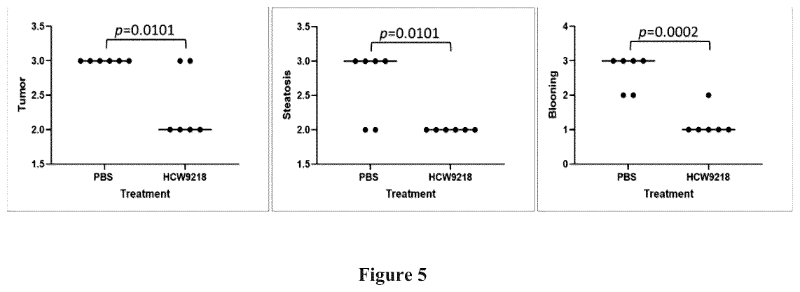

B6C3F1 male mice were purchased from The Jackson Laboratory. The mice were divided into two groups as follows: saline control group (n=6) and TGFRt15-TGFRs group (n=6). All mice (14-day-old) were peritoneally treated with DEN (1 mg/kg, diethylnitrosamine) on study day zero (SDO). CCl₄ (0.2 mL/kg, carbon tetrachloride) was peritoneally injected into the mice at 8 weeks of age (SD42) and continued to treat at twice a week for up to 14 additional weeks. TGFRt15-TGFRs was subcutaneously injected (3 mg/kg) on SD43 and SD71. The treated mice were euthanized on SD161 and the livers were harvested and embedded in 4% formalin. The liver sections were stained with hematoxylin and eosin. Tumor, steatosis and hepatocellular ballooning were examined under the light microscope. The severity of liver damage was expressed as mild (1), moderate (2) and extensive (3). Statistical analyses were performed using GraphPad Prism 9 by unpaired t test. For each test, a P value of less than 0.05 was considered statistically significant. As shown in FIG. 5, TGFRt15-TGFRs significantly inhibited liver tumor development and growth, steatosis and hepatocellular ballooning induced by DEN and CCl₄ in B6C3F1 mice.

### Example 4. Treatment of Advanced Solid Tumors

### Patients and Patient Disposition

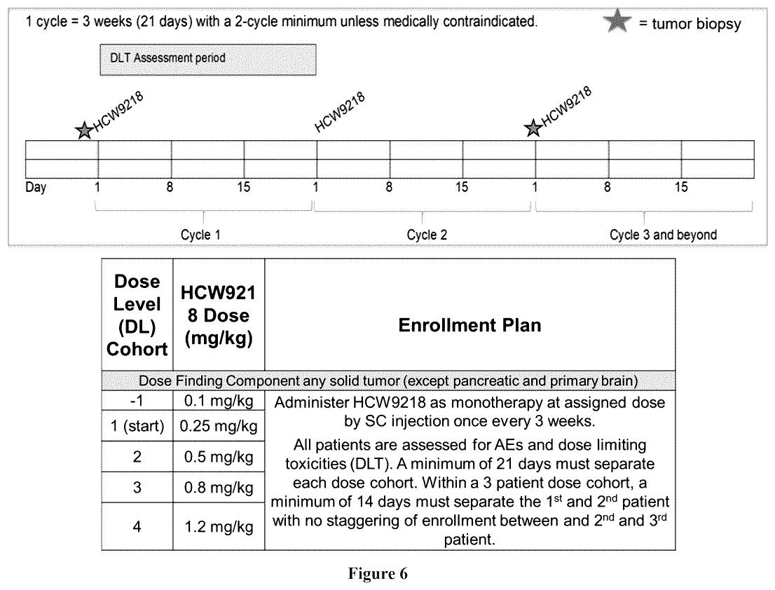

TGFRt15-TGFRs (HCW9218) was administered subcutaneously in the outpatient setting to subjects with chemotherapy refractory/resistant advanced solid tumors. Treatment was given once every 3 weeks for a minimum of 2 cycles (FIG. 6). TGFRt15-TGFRs (HCW9218) dose ranges were from 0.25 mg/kg (DL1) to 1.2 mg/kg (DL4). Correlative objectives include immunogenicity, pharmacokinetic (PK) profiles of TGFRt15-TGFRs (HCW9218), lymphocyte number, phenotype and function by flow cytometry analysis.

Patients have failed at least 2 prior lines of therapy given either in the recurrent or metastatic setting and must be refractory to or intolerant of existing therapy.

Fifteen patients were enrolled. Five patients remain on TGFRt15-TGFRs (HCW9218). Four solid tumor indications were represented. Median number of cycles received was 3. Baseline characteristics are summarized in Table 1.

TABLE 1
Patient Demographics

| | Patients (n = 15) |
|---|---|
| Age, years, median (range) | 56 (39-70) |
| Sex, male/female (%) | 8/7, (53%) |
| ECOG PS | |
| 0 | 7 (47%) |
| 1 | 8 (53%) |
| Disease sites, n(%) | |
| Ovarian | 6 (40%) |
| Colon | 4 (27%) |
| Rectal | 3 (20%) |
| Liver | 2 (13%) |
| # previous lines of therapy, n (%) | |
| 2 | 2 (13%) |
| >4 | 13 (87%) |

### Tumor Responses

Stable disease was observed in 4 heavily pretreated advanced solid tumor patients (2 ovarian, 1 rectal, 1 liver). Repeated TGFRt15-TGFRs (HCW9218) administration (up to 6 cycles) resulted in immune cell activation, proliferation, and infiltration into the tumor microenvironment without causing unacceptable toxicity. TGFRt15-TGFRs (HCW9218) treatment presents a promising approach to enhancing the antitumor activity of immune checkpoint inhibitors in patients with solid tumors.

### Toxicity

During the dose escalation phase of the trial, there were no DLT's encountered. At the 4^{th} DL expansion, there was 1 DLT (Gr 3 ascites) that did not trigger the stopping rules. Treatment related AE's at least possibly related to the study medication are summarized in Table 2.

TABLE 2
Most Frequent TRAE's in N = 15 patients

| Toxicity Summary | Any Grade | Grade ≥3 N, (%) |
|---|---|---|
| Total Number of | 402 | 40 (9.9%) |
| TRAEs Experienced | | |
| Total % of TRAEs | 15 (100%) | 14 (93%) |
| experienced by the patients | | |

| TRAE, % of | Any Grade | Grade ≥3 |
|---|---|---|
| total TRAEs | Count, (% of pts) | Count, (% of pts) |
| Injection site | 72 (100%) | 1 (7%) |
| rxn (18.1%) | | |
| Flu like | 39 (87%) | 0 |
| symptoms (9.7%) | | |
| Lymphocyte count | 35 (93%) | 21 (74%) |
| decreased (16.4%) | | |

### Correlative and Immune Data

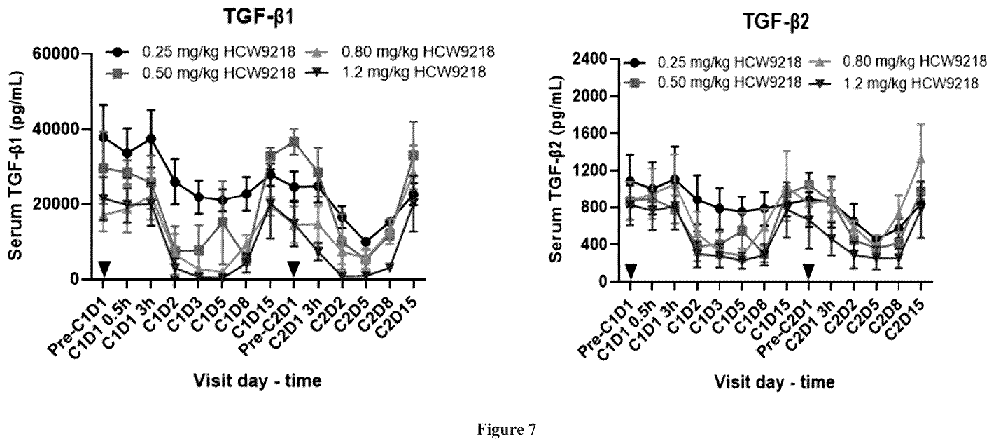

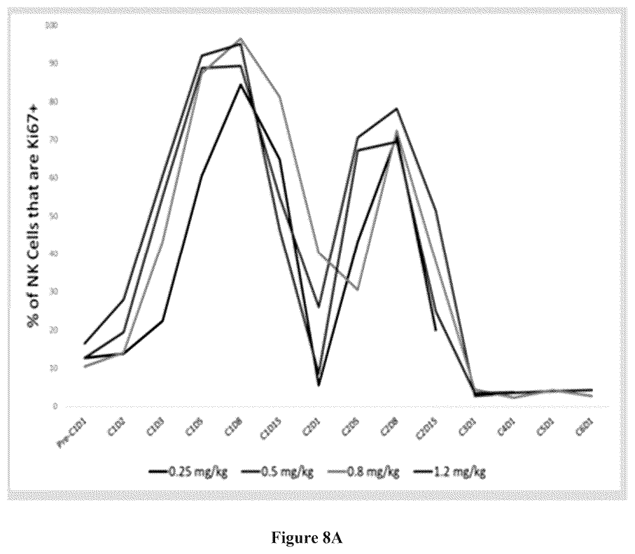

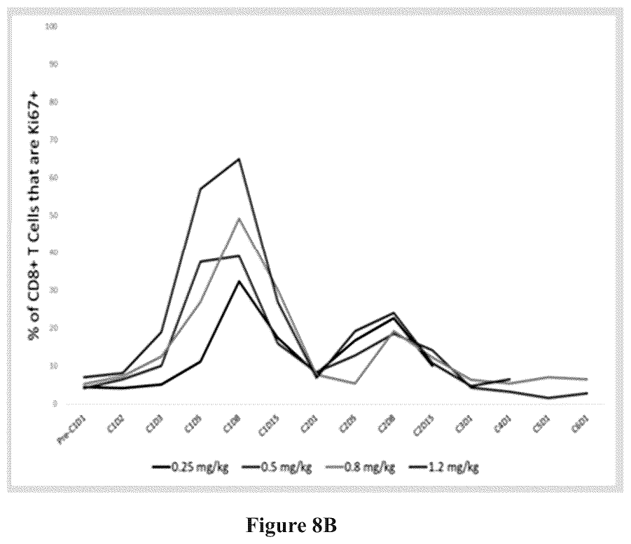

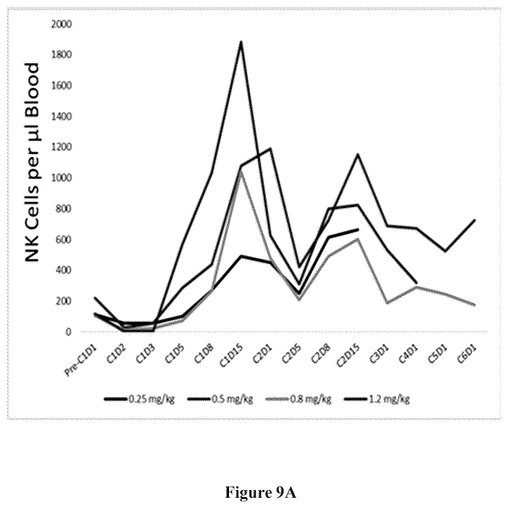

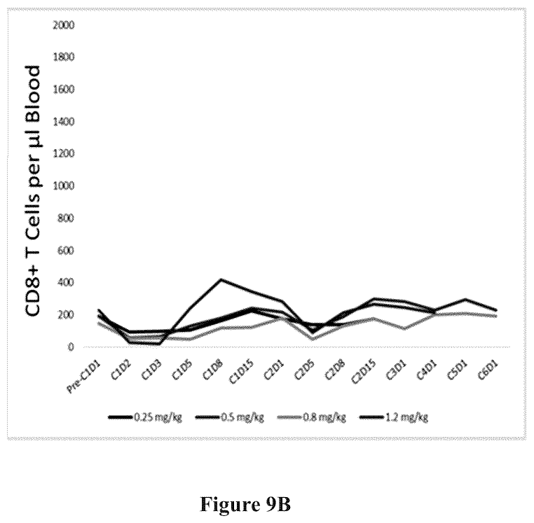

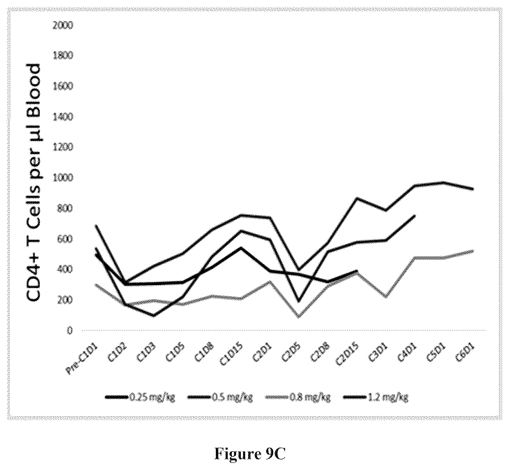

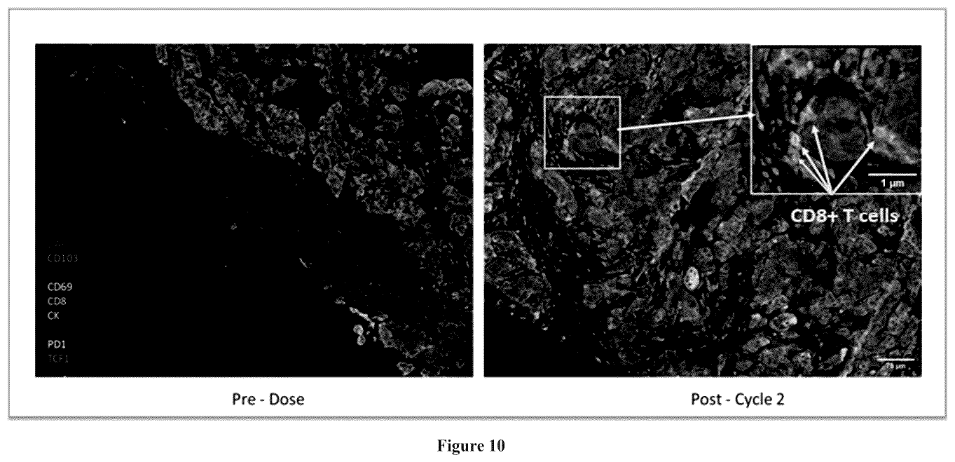

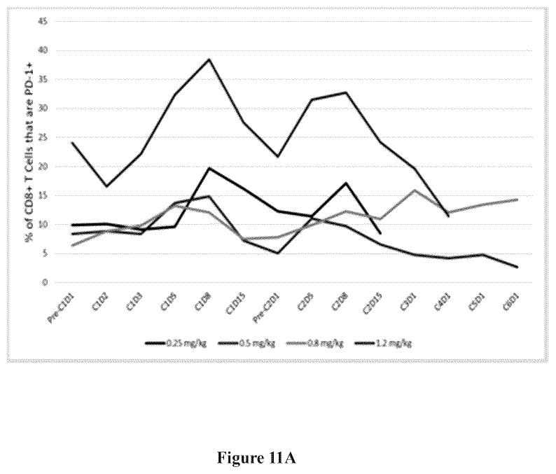

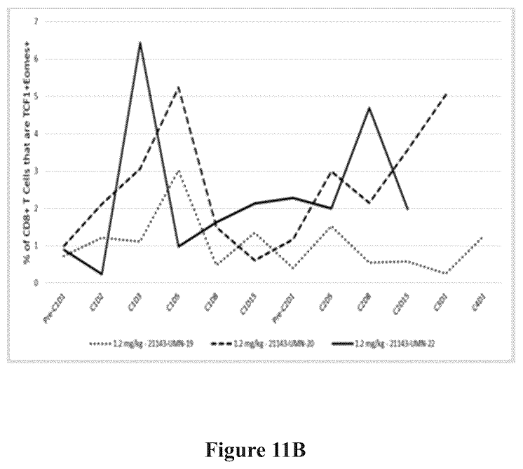

Neutralization of TGF-β1 and TGF-β2 by dose level was measured, wherein TGFRt15-TGFRs (HCW9218) dose-dependent reduction in serum TGF-β 1 and TGF-β2 levels (to baseline at >0.5 mg/kg HCW9218) were observed (FIG. 7). Percentage of Ki67⁺ NK cells and CD8⁺ T cells were measured by flow cytometry (FIGS. 8A-8B). All subjects had a robust proliferation of blood NK cells, ranging from 77% to 97% Ki67-positivity by Day 8 after dosing for each treatment cycle. HCW9218-mediated increases in blood NK cell percentages and counts were also observed. Treatment induction for blood CD8⁺ T cell proliferation was also observed. Responses were sustained through Day 15, a biological effect beyond that previously observed for other IL-15 agonists. Absolute number of NK cells, CD8⁺, and CD4⁺ T cells were also measured (FIGS. 9A-9C). Immune cell staining was performed in pre- and post-treatment tumor biopsy specimens, wherein TGFRt15-TGFRs (HCW9218) treatment induced CD8⁺ T cells trafficking to tumor in an ovarian cancer patient with stable disease (FIG. 10). Similar results were seen in tumor biopsies of two other patients (ovarian and rectal cancer) with stable disease. Furthermore, the presence of exhausted (PD1⁺) and Tpex (TCF1⁺) CD8⁺ T cells was evaluated using time of flight mass cytometry (CyTOF) using Maxpar Direct Immune Profiling assay with a NK cell expansion panel (for dose escalation) or a custom CD8⁺ T cell expansion panel (for the extension cohort) and then analyzed with Maxpar Pathsetter software (FIGS. 11A-11B).

Repeated TGFRt15-TGFRs (HCW9218) administration at up to 1.2 mg/kg was well tolerated by heavily pretreated advanced solid tumor patients. Results showed that TGFRt15-TGFRs (HCW9218) treatment resulted in NK cell and CD8⁺ T cell (including Tpex and memory CD8⁺ T cells) activation, proliferation, and infiltration into the tumor microenvironment which correlated with disease stabilization. TGFRt15-TGFRs (HCW9218) also reduced TGF-β levels in tumors (mouse tumor models) and blood (mouse and human clinical studies).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating an unresectable, advanced, recurrent, and/or metastatic solid cancer or solid tumor in a subject, the method comprising administering to the subject two or more doses of a multi-chain chimeric polypeptide, wherein the time between administration of any two consecutive doses of the two or more doses is 15 days to 27 days, wherein the multi-chain chimeric polypeptide comprises:
(a) a first chimeric polypeptide comprising:
(i) a first target-binding domain;
(ii) a soluble tissue factor domain comprising a sequence that is at least 90% identical to SEQ ID NO: 1; and
(iii) a first domain of a pair of affinity domains comprising a sequence that is at least 90% identical to SEQ ID NO:13;
(b) a second chimeric polypeptide comprising:
(i) a second domain of a pair of affinity domains comprising a sequence that is at least 90% identical to SEQ ID NO:11; and
(ii) a second target-binding domain,
wherein:
the first chimeric polypeptide and the second chimeric polypeptide associate through the binding of the first domain and the second domain of the pair of affinity domains; and the first target-binding domain and the second target-binding domain each comprises a soluble TGF-β receptor II (TGF-βRII) and each comprises a sequence that is at least 90% identical to SEQ ID NO:4.

2. The method of claim 1, wherein the subject has previously received a previous treatment with a standard first or later line of systemic therapy for a solid tumor, and the subject's solid tumor had recurred or progressed and/or the subject was intolerant to the previous treatment.

3. The method of claim 1, wherein the subject has been previous identified or diagnosed as having an unresectable, advanced, recurrent, and/or metastatic solid tumor or unresectable, advanced, recurrent, and/or metastatic solid cancer.

4. The method of claim 1, wherein the solid cancer or solid tumor is selected from the group consisting of: bladder cancer, gastric cancer, breast cancer, cervical cancer, colorectal cancer, endometrial cancer, kidney cancer, lip cancer, liver cancer, melanoma, rhabdomyosarcoma, Ewing sarcoma, mesothelioma, lung cancer, non-small cell lung cancer, non-melanoma skin cancer, oral cancer, ovarian cancer, pancreatic cancer, sarcoma, osteosarcoma, glioblastoma, neuroblastoma, small cell lung cancer, thyroid cancer, retinoblastoma, urothelial carcinoma, renal cell carcinoma, esophageal cancer, prostate cancer, squamous cell head and neck carcinoma, hepatocellular cancer, testicular cancer, penile cancer, uterine cancer, vaginal cancer, gallbladder cancer, Merkel cell carcinoma, gastroesophageal junction cancer, microsatellite instability-high (MSI-H) solid tumor, a mismatch repair deficient (dMMR) solid tumor, and tumor mutational burden-high (TMB-H) solid tumor.

5. The method of claim 1, wherein the cancer is ovarian cancer, colorectal cancer or liver cancer.

6. The method of claim 1, wherein each of the two or more doses is 0.2 mg/kg to 1.3 mg/kg.

7. The method of claim 1, wherein each of the two or more doses is 0.25 mg/kg to 1.2 mg/kg.

8. The method of claim 1, wherein each of the two or more doses is 20 mg to 150 mg.

9. The method of claim 1, wherein the two or more doses are formulated for subcutaneous or intravenous administration.

10. The method of claim 1, wherein the first target-binding domain comprises a sequence of SEQ ID NO: 4.

11. The method of claim 1, wherein the first chimeric polypeptide comprises a sequence that is at least 80% identical to SEQ ID NO:6.

12. The method of claim 11, wherein the first chimeric polypeptide comprises a sequence that is at least 90% identical to SEQ ID NO:6.

13. The method of claim 12, wherein the first chimeric polypeptide comprises a sequence of SEQ ID NO:6.

14. The method of claim 1, wherein the second target-binding domain comprises a sequence of SEQ ID NO:4.

15. The method of claim 1, wherein the second chimeric polypeptide comprises a sequence that is at least 80% identical to SEQ ID NO:5.

16. The method of claim 15, wherein the first chimeric polypeptide comprises a sequence that is at least 80% identical to SEQ ID NO:6.

17. The method of claim 16, wherein the second chimeric polypeptide comprises a sequence that is at least 90% identical to SEQ ID NO:5.

18. The method of claim 17, wherein the second chimeric polypeptide comprises a sequence of SEQ ID NO:5.

19. The method of claim 18, wherein the first chimeric polypeptide comprises a sequence of SEQ ID NO:6.


SEQUENCE LISTING (38 sequences)

SEQ ID NO: 1 - protein, 219 aa, Homo sapiens
SGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRE

SEQ ID NO: 2 - protein, 136 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 3 - protein, 15 aa, synthetic construct
GGGGSGGGGSGGGGS

SEQ ID NO: 4 - protein, 287 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 5 - protein, 352 aa, synthetic construct
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDITCPPPMSVEHADIWVKSYSLYSRERYICNSGFKRKAGTSSLTECVLNKATNVAHWTTPSLKCIR

SEQ ID NO: 6 - protein, 620 aa, synthetic construct
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDSGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRENWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 7 - protein, 638 aa, synthetic construct
MKWVTFISLLFLFSSAYSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDSGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRENWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 8 - protein, 370 aa, synthetic construct
MKWVTFISLLFLFSSAYSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDITCPPPMSVEHADIWVKSYSLYSRERYICNSGFKRKAGTSSLTECVLNKATNVAHWTTPSLKCIR

SEQ ID NO: 9 - DNA, 657 bp, Homo sapiens
  source 1..657: mol_type = genomic DNA
agcggcacaaccaacacagtcgctgcctataacctcacttggaagagcaccaacttcaaaaccatcctcgaatgggaacccaaacccgttaaccaagtttacaccgtgcagatcagcaccaagtccggcgactggaagtccaaatgtttctataccaccgacaccgagtgcgatctcaccgatgagatcgtgaaagatgtgaaacagacctacctcgcccgggtgtttagctaccccgccggcaatgtggagagcactggttccgctggcgagcctttatacgagaacagccccgaatttaccccttacctcgagaccaatttaggacagcccaccatccaaagctttgagcaagttggcacaaaggtgaatgtgacagtggaggacgagcggactttagtgcggcggaacaacacctttctcagcctccgggatgtgttcggcaaagatttaatctacacactgtattactggaagtcctcttcctccggcaagaagacagctaaaaccaacacaaacgagtttttaatcgacgtggataaaggcgaaaactactgtttcagcgtgcaagctgtgatcccctcccggaccgtgaataggaaaagcaccgatagccccgttgagtgcatgggccaagaaaagggcgagttccgggag

SEQ ID NO: 10 - DNA, 45 bp, synthetic construct
  source 1..45: mol_type = other DNA
ggcggtggaggatccggaggaggtggctccggcggcggaggatct

SEQ ID NO: 11 - protein, 65 aa, Homo sapiens
ITCPPPMSVEHADIWVKSYSLYSRERYICNSGFKRKAGTSSLTECVLNKATNVAHWTTPSLKCIR

SEQ ID NO: 12 - DNA, 195 bp, Homo sapiens
  source 1..195: mol_type = genomic DNA
attacatgcccccctcccatgagcgtggagcacgccgacatctgggtgaagagctatagcctctacagccgggagaggtatatctgtaacagcggcttcaagaggaaggccggcaccagcagcctcaccgagtgcgtgctgaataaggctaccaacgtggctcactggacaacaccctctttaaagtgcatccgg

SEQ ID NO: 13 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 14 - DNA, 342 bp, Homo sapiens
  source 1..342: mol_type = genomic DNA
aactgggtgaacgtcatcagcgatttaaagaagatcgaagatttaattcagtccatgcatatcgacgccactttatacacagaatccgacgtgcacccctcttgtaaggtgaccgccatgaaatgttttttactggagctgcaagttatctctttagagagcggagacgctagcatccacgacaccgtggagaatttaatcattttagccaataactctttatccagcaacggcaacgtgacagagtccggctgcaaggagtgcgaagagctggaggagaagaacatcaaggagtttctgcaatcctttgtgcacattgtccagatgttcatcaatacctcc

SEQ ID NO: 15 - protein, 18 aa, synthetic construct
MKWVTFISLLFLFSSAYS

SEQ ID NO: 16 - DNA, 54 bp, synthetic construct
  source 1..54: mol_type = other DNA
atgaaatgggtgacctttatttctttactgttcctctttagcagcgcctactcc

SEQ ID NO: 17 - DNA, 54 bp, synthetic construct
  source 1..54: mol_type = other DNA
atgaagtgggtcacatttatctctttactgttcctcttctccagcgcctacagc

SEQ ID NO: 18 - DNA, 54 bp, synthetic construct; identical to SEQ ID NO: 16
  source 1..54: mol_type = other DNA

SEQ ID NO: 19 - protein, 16 aa, synthetic construct
MKCLLYLAFLFLGVNC

SEQ ID NO: 20 - protein, 58 aa, synthetic construct
MGQIVTMFEALPHIIDEVINIVIIVLIIITSIKAVYNFATCGILALVSFLFLAGRSCG

SEQ ID NO: 21 - protein, 97 aa, synthetic construct
MPNHQSGSPTGSSDLLLSGKKQRPHLALRRKRRREMRKINRKVRRMNLAPIKEKTAWQHLQALISEAEEVLKTSQTPQNSLTLFLALLSVLGPPVTG

SEQ ID NO: 22 - protein, 30 aa, synthetic construct
MDSKGSSQKGSRLLLLLVVSNLLLCQGVVS

SEQ ID NO: 23 - DNA, 408 bp, Homo sapiens
  source 1..408: mol_type = genomic DNA
atccccccccatgtgcaaaagagcgtgaacaacgatatgatcgtgaccgacaacaacggcgccgtgaagtttccccagctctgcaagttctgcgatgtcaggttcagcacctgcgataatcagaagtcctgcatgtccaactgcagcatcacctccatctgcgagaagccccaagaagtgtgcgtggccgtgtggcggaaaaatgacgagaacatcaccctggagaccgtgtgtcacgaccccaagctcccttatcacgacttcattctggaggacgctgcctcccccaaatgcatcatgaaggagaagaagaagcccggagagaccttctttatgtgttcctgtagcagcgacgagtgtaacgacaacatcatcttcagcgaagagtacaacaccagcaaccctgat

SEQ ID NO: 24 - DNA, 408 bp, Homo sapiens
  source 1..408: mol_type = genomic DNA
attcctccccacgtgcagaagagcgtgaataatgacatgatcgtgaccgataacaatggcgccgtgaaatttccccagctgtgcaaattctgcgatgtgaggttttccacctgcgacaaccagaagtcctgtatgagcaactgctccatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgaatgcaacgacaatatcatctttagcgaggaatacaataccagcaaccccgac

SEQ ID NO: 25 - DNA, 861 bp, Homo sapiens
  source 1..861: mol_type = genomic DNA
atccccccccatgtgcaaaagagcgtgaacaacgatatgatcgtgaccgacaacaacggcgccgtgaagtttccccagctctgcaagttctgcgatgtcaggttcagcacctgcgataatcagaagtcctgcatgtccaactgcagcatcacctccatctgcgagaagccccaagaagtgtgcgtggccgtgtggcggaaaaatgacgagaacatcaccctggagaccgtgtgtcacgaccccaagctcccttatcacgacttcattctggaggacgctgcctcccccaaatgcatcatgaaggagaagaagaagcccggagagaccttctttatgtgttcctgtagcagcgacgagtgtaacgacaacatcatcttcagcgaagagtacaacaccagcaaccctgatggaggtggcggatccggaggtggaggttctggtggaggtgggagtattcctccccacgtgcagaagagcgtgaataatgacatgatcgtgaccgataacaatggcgccgtgaaatttccccagctgtgcaaattctgcgatgtgaggttttccacctgcgacaaccagaagtcctgtatgagcaactgctccatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgaatgcaacgacaatatcatctttagcgaggaatacaataccagcaaccccgac

SEQ ID NO: 26 - DNA, 1860 bp, synthetic construct
  source 1..1860: mol_type = other DNA
atccccccccatgtgcaaaagagcgtgaacaacgatatgatcgtgaccgacaacaacggcgccgtgaagtttccccagctctgcaagttctgcgatgtcaggttcagcacctgcgataatcagaagtcctgcatgtccaactgcagcatcacctccatctgcgagaagccccaagaagtgtgcgtggccgtgtggcggaaaaatgacgagaacatcaccctggagaccgtgtgtcacgaccccaagctcccttatcacgacttcattctggaggacgctgcctcccccaaatgcatcatgaaggagaagaagaagcccggagagaccttctttatgtgttcctgtagcagcgacgagtgtaacgacaacatcatcttcagcgaagagtacaacaccagcaaccctgatggaggtggcggatccggaggtggaggttctggtggaggtgggagtattcctccccacgtgcagaagagcgtgaataatgacatgatcgtgaccgataacaatggcgccgtgaaatttccccagctgtgcaaattctgcgatgtgaggttttccacctgcgacaaccagaagtcctgtatgagcaactgctccatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgaatgcaacgacaatatcatctttagcgaggaatacaataccagcaaccccgacagcggcacaaccaacacagtcgctgcctataacctcacttggaagagcaccaacttcaaaaccatcctcgaatgggaacccaaacccgttaaccaagtttacaccgtgcagatcagcaccaagtccggcgactggaagtccaaatgtttctataccaccgacaccgagtgcgatctcaccgatgagatcgtgaaagatgtgaaacagacctacctcgcccgggtgtttagctaccccgccggcaatgtggagagcactggttccgctggcgagcctttatacgagaacagccccgaatttaccccttacctcgagaccaatttaggacagcccaccatccaaagctttgagcaagttggcacaaaggtgaatgtgacagtggaggacgagcggactttagtgcggcggaacaacacctttctcagcctccgggatgtgttcggcaaagatttaatctacacactgtattactggaagtcctcttcctccggcaagaagacagctaaaaccaacacaaacgagtttttaatcgacgtggataaaggcgaaaactactgtttcagcgtgcaagctgtgatcccctcccggaccgtgaataggaaaagcaccgatagccccgttgagtgcatgggccaagaaaagggcgagttccgggagaactgggtgaacgtcatcagcgatttaaagaagatcgaagatttaattcagtccatgcatatcgacgccactttatacacagaatccgacgtgcacccctcttgtaaggtgaccgccatgaaatgttttttactggagctgcaagttatctctttagagagcggagacgctagcatccacgacaccgtggagaatttaatcattttagccaataactctttatccagcaacggcaacgtgacagagtccggctgcaaggagtgcgaagagctggaggagaagaacatcaaggagtttctgcaatcctttgtgcacattgtccagatgttcatcaatacctcc

SEQ ID NO: 27 - DNA, 1914 bp, synthetic construct
  source 1..1914: mol_type = other DNA
atgaagtgggtgaccttcatcagcctgctgttcctgttctccagcgcctactccatccccccccatgtgcaaaagagcgtgaacaacgatatgatcgtgaccgacaacaacggcgccgtgaagtttccccagctctgcaagttctgcgatgtcaggttcagcacctgcgataatcagaagtcctgcatgtccaactgcagcatcacctccatctgcgagaagccccaagaagtgtgcgtggccgtgtggcggaaaaatgacgagaacatcaccctggagaccgtgtgtcacgaccccaagctcccttatcacgacttcattctggaggacgctgcctcccccaaatgcatcatgaaggagaagaagaagcccggagagaccttctttatgtgttcctgtagcagcgacgagtgtaacgacaacatcatcttcagcgaagagtacaacaccagcaaccctgatggaggtggcggatccggaggtggaggttctggtggaggtgggagtattcctccccacgtgcagaagagcgtgaataatgacatgatcgtgaccgataacaatggcgccgtgaaatttccccagctgtgcaaattctgcgatgtgaggttttccacctgcgacaaccagaagtcctgtatgagcaactgctccatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgaatgcaacgacaatatcatctttagcgaggaatacaataccagcaaccccgacagcggcacaaccaacacagtcgctgcctataacctcacttggaagagcaccaacttcaaaaccatcctcgaatgggaacccaaacccgttaaccaagtttacaccgtgcagatcagcaccaagtccggcgactggaagtccaaatgtttctataccaccgacaccgagtgcgatctcaccgatgagatcgtgaaagatgtgaaacagacctacctcgcccgggtgtttagctaccccgccggcaatgtggagagcactggttccgctggcgagcctttatacgagaacagccccgaatttaccccttacctcgagaccaatttaggacagcccaccatccaaagctttgagcaagttggcacaaaggtgaatgtgacagtggaggacgagcggactttagtgcggcggaacaacacctttctcagcctccgggatgtgttcggcaaagatttaatctacacactgtattactggaagtcctcttcctccggcaagaagacagctaaaaccaacacaaacgagtttttaatcgacgtggataaaggcgaaaactactgtttcagcgtgcaagctgtgatcccctcccggaccgtgaataggaaaagcaccgatagccccgttgagtgcatgggccaagaaaagggcgagttccgggagaactgggtgaacgtcatcagcgatttaaagaagatcgaagatttaattcagtccatgcatatcgacgccactttatacacagaatccgacgtgcacccctcttgtaaggtgaccgccatgaaatgttttttactggagctgcaagttatctctttagagagcggagacgctagcatccacgacaccgtggagaatttaatcattttagccaataactctttatccagcaacggcaacgtgacagagtccggctgcaaggagtgcgaagagctggaggagaagaacatcaaggagtttctgcaatcctttgtgcacattgtccagatgttcatcaatacctcc

SEQ ID NO: 28 - DNA, 1056 bp, synthetic construct
  source 1..1056: mol_type = other DNA
atccccccccatgtgcaaaagagcgtgaacaacgatatgatcgtgaccgacaacaacggcgccgtgaagtttccccagctctgcaagttctgcgatgtcaggttcagcacctgcgataatcagaagtcctgcatgtccaactgcagcatcacctccatctgcgagaagccccaagaagtgtgcgtggccgtgtggcggaaaaatgacgagaacatcaccctggagaccgtgtgtcacgaccccaagctcccttatcacgacttcattctggaggacgctgcctcccccaaatgcatcatgaaggagaagaagaagcccggagagaccttctttatgtgttcctgtagcagcgacgagtgtaacgacaacatcatcttcagcgaagagtacaacaccagcaaccctgatggaggtggcggatccggaggtggaggttctggtggaggtgggagtattcctccccacgtgcagaagagcgtgaataatgacatgatcgtgaccgataacaatggcgccgtgaaatttccccagctgtgcaaattctgcgatgtgaggttttccacctgcgacaaccagaagtcctgtatgagcaactgctccatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgaatgcaacgacaatatcatctttagcgaggaatacaataccagcaaccccgacattacatgcccccctcccatgagcgtggagcacgccgacatctgggtgaagagctatagcctctacagccgggagaggtatatctgtaacagcggcttcaagaggaaggccggcaccagcagcctcaccgagtgcgtgctgaataaggctaccaacgtggctcactggacaacaccctctttaaagtgcatccgg

SEQ ID NO: 29 - DNA, 1110 bp, synthetic construct
  source 1..1110: mol_type = other DNA
atgaagtgggtgaccttcatcagcctgctgttcctgttctccagcgcctactccatccccccccatgtgcaaaagagcgtgaacaacgatatgatcgtgaccgacaacaacggcgccgtgaagtttccccagctctgcaagttctgcgatgtcaggttcagcacctgcgataatcagaagtcctgcatgtccaactgcagcatcacctccatctgcgagaagccccaagaagtgtgcgtggccgtgtggcggaaaaatgacgagaacatcaccctggagaccgtgtgtcacgaccccaagctcccttatcacgacttcattctggaggacgctgcctcccccaaatgcatcatgaaggagaagaagaagcccggagagaccttctttatgtgttcctgtagcagcgacgagtgtaacgacaacatcatcttcagcgaagagtacaacaccagcaaccctgatggaggtggcggatccggaggtggaggttctggtggaggtgggagtattcctccccacgtgcagaagagcgtgaataatgacatgatcgtgaccgataacaatggcgccgtgaaatttccccagctgtgcaaattctgcgatgtgaggttttccacctgcgacaaccagaagtcctgtatgagcaactgctccatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgaatgcaacgacaatatcatctttagcgaggaatacaataccagcaaccccgacattacatgcccccctcccatgagcgtggagcacgccgacatctgggtgaagagctatagcctctacagccgggagaggtatatctgtaacagcggcttcaagaggaaggccggcaccagcagcctcaccgagtgcgtgctgaataaggctaccaacgtggctcactggacaacaccctctttaaagtgcatccgg

SEQ ID NO: 30 - protein, 352 aa, synthetic construct; identical to SEQ ID NO: 5

SEQ ID NO: 31 - DNA, 1056 bp, synthetic construct; identical to SEQ ID NO: 28
  source 1..1056: mol_type = other DNA

SEQ ID NO: 32 - DNA, 1110 bp, synthetic construct; identical to SEQ ID NO: 29
  source 1..1110: mol_type = other DNA

SEQ ID NO: 33 - protein, 620 aa, synthetic construct
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDSGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRENWVNVISNLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 34 - DNA, 1860 bp, synthetic construct
  source 1..1860: mol_type = other DNA
atcccaccgcacgttcagaagtcggtgaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacggaggtggcggatccggaggtggaggttctggtggaggtgggagtattcctccccacgtgcagaagagcgtgaataatgacatgatcgtgaccgataacaatggcgccgtgaaatttccccagctgtgcaaattctgcgatgtgaggttttccacctgcgacaaccagaagtcctgtatgagcaactgctccatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgaatgcaacgacaatatcatctttagcgaggaatacaataccagcaaccccgactcaggcactacaaatactgtggcagcatataatttaacttggaaatcaactaatttcaagacaattttggagtgggaacccaaacccgtcaatcaagtctacactgttcaaataagcactaagtcaggagattggaaaagcaaatgcttttacacaacagacacagagtgtgacctcaccgacgagattgtgaaggatgtgaagcagacgtacttggcacgggtcttctcctacccggcagggaatgtggagagcaccggttctgctggggagcctctgtatgagaactccccagagttcacaccttacctggagacaaacctcggacagccaacaattcagagttttgaacaggtgggaacaaaagtgaatgtgaccgtagaagatgaacggactttagtcagaaggaacaacactttcctaagcctccgggatgtttttggcaaggacttaatttatacactttattattggaaatcttcaagttcaggaaagaaaacagccaaaacaaacactaatgagtttttgattgatgtggataaaggagaaaactactgtttcagtgttcaagcagtgattccctcccgaacagttaaccggaagagtacagacagcccggtagagtgtatgggccaggagaaaggggaattcagagaaaactgggtgaatgtaataagtaatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 35 - protein, 637 aa, synthetic construct
MGVKVLFALICIAVAEAIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDSGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRENWVNVISNLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 36 - DNA, 1911 bp, synthetic construct
  source 1..1911: mol_type = other DNA
atgggagtgaaagttctttttgcccttatttgtattgctgtggccgaggccatcccaccgcacgttcagaagtcggtgaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacggaggtggcggatccggaggtggaggttctggtggaggtgggagtattcctccccacgtgcagaagagcgtgaataatgacatgatcgtgaccgataacaatggcgccgtgaaatttccccagctgtgcaaattctgcgatgtgaggttttccacctgcgacaaccagaagtcctgtatgagcaactgctccatcacctccatctgtgagaagcctcaggaggtgtgcgtggctgtctggcggaagaatgacgagaatatcaccctggaaaccgtctgccacgatcccaagctgccctaccacgatttcatcctggaagacgccgccagccctaagtgcatcatgaaagagaaaaagaagcctggcgagacctttttcatgtgctcctgcagcagcgacgaatgcaacgacaatatcatctttagcgaggaatacaataccagcaaccccgactcaggcactacaaatactgtggcagcatataatttaacttggaaatcaactaatttcaagacaattttggagtgggaacccaaacccgtcaatcaagtctacactgttcaaataagcactaagtcaggagattggaaaagcaaatgcttttacacaacagacacagagtgtgacctcaccgacgagattgtgaaggatgtgaagcagacgtacttggcacgggtcttctcctacccggcagggaatgtggagagcaccggttctgctggggagcctctgtatgagaactccccagagttcacaccttacctggagacaaacctcggacagccaacaattcagagttttgaacaggtgggaacaaaagtgaatgtgaccgtagaagatgaacggactttagtcagaaggaacaacactttcctaagcctccgggatgtttttggcaaggacttaatttatacactttattattggaaatcttcaagttcaggaaagaaaacagccaaaacaaacactaatgagtttttgattgatgtggataaaggagaaaactactgtttcagtgttcaagcagtgattccctcccgaacagttaaccggaagagtacagacagcccggtagagtgtatgggccaggagaaaggggaattcagagaaaactgggtgaatgtaataagtaatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct

SEQ ID NO: 37 - protein, 114 aa, synthetic construct
NWVNVISNLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 38 - DNA, 342 bp, synthetic construct
  source 1..342: mol_type = other DNA
aactgggtgaatgtaataagtaatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttct